(19) United States
(12) Patent Application Publication
(10) Pub. No.: US 2026/0021160 A1
(43) Pub. Date: Jan. 22, 2026

(54) REGENERATING FUNCTIONAL NEURONS FOR TREATMENT OF SPINAL CORD INJURY AND ALS

(71) Applicant: The Penn State Research Foundation, University Park, PA (US)

(72) Inventor: Gong CHEN, Guangzhou (CN)

(73) Assignee: The Penn State Research Foundation, University Park, PA (US)

(21) Appl. No.: 18/486,750

(22) Filed: Oct. 13, 2023

Related U.S. Application Data
(63) Continuation of application No. 17/072,951, filed on Oct. 16, 2020, now abandoned.
(60) Provisional application No. 62/916,713, filed on Oct. 17, 2019.
(60) Provisional application No. 63/040,989, filed on Jun. 18, 2020.

(51) Int. Cl. A61K 38/17; A61P 25/00; C07K 14/47; C12N 5/0793; C12N 15/86

(52) U.S. Cl. CPC A61K 38/17; A61P 25/00; C07K 14/4702; C12N 5/0619; C12N 15/86; C12N 2750/14141

(57) ABSTRACT

This document provides methods and materials involved in treating mammals having a spinal cord injury (SCI). For example, methods and materials for administering a composition containing exogenous nucleic acid encoding a NeuroD1polypeptide (or a biologically active fragment thereof) alone or in combination with a Dlx2 polypeptide (or a biologically active fragment thereof) to a mammal having SCI are provided. This document also provides methods and materials involved in treating mammals having amyotrophic lateral sclerosis (ALS). For example, methods and materials for administering a composition containing exogenous nucleic acid encoding a NeuroD1 polypeptide (or a biologically active fragment thereof) alone or in combination with an Isl1 polypeptide (or a biologically active fragment thereof) to a mammal having ALS are provided.

### CROSS-REFERENCE TO RELATED APPLICATIONS

This application claims the benefit of U.S. Application Ser. No. 63/040,989, filed on Jun. 18, 2020, and U.S. Application Ser. No. 62/916,713, filed on Oct. 17, 2019, the contents of these aforementioned applications are fully incorporated herein by reference.

### GOVERNMENT SUPPORT

This invention was made with government support under Grant No. W81XWH-16-1-0163 awarded by the United States Army/MRMC. The Government has certain rights in the invention.

### BACKGROUND

### 1. Technical Field

This document relates to methods and materials involved in treating mammals having a spinal cord injury (SCI). For example, this document provides methods and materials for administering a composition containing exogenous nucleic acid encoding a NeuroD1polypeptide (or a biologically active fragment thereof) alone or in combination with exogenous nucleic acid encoding a Dlx2 polypeptide (or a biologically active fragment thereof) to a mammal having SCI. This document also relates to methods and materials involved in treating mammals having amyotrophic lateral sclerosis (ALS). For example, this document provides methods and materials for administering a composition containing exogenous nucleic acid encoding a NeuroD1 polypeptide (or a biologically active fragment thereof) to a mammal having ALS. As another example, this document provides methods and materials for administering a composition containing exogenous nucleic acid encoding a NeuroD1 polypeptide (or a biologically active fragment thereof) alone or in combination with exogenous nucleic acid encoding an Isl1 polypeptide (or a biologically active fragment thereof) to a mammal having ALS.

### 2. Background Information

Spinal cord injury (SCI) is a devastating central nervous system (CNS) disorder and often leads to loss of motor and sensory functions below the injury site, even paralysis depending on the severity of the injury (Adams and Hicks, Spinal Cord, 43:577-586 (2005)). The pathophysiological process after SCI is rather complex, resulting in neuronal loss, neuroinflammation, demyelination, and Wallerian degeneration of the axons (Norenberg et al., J. Neurotrauma, 21:429-440 (2004)). Reactive astrogliosis is common to CNS injury, and particularly severe after SCI. Resident astrocytes react to injury-induced cytokines and dramatically upregulate the expression of a number of proteins such as the astrocytic marker GFAP and the neural progenitor markers Nestin and Vimentin (Sofroniew, Trends Neurosci., 32:638-647 (2009)). These reactive astrocytes also become proliferative and hypertrophic in cell morphology. In the acute phase of SCI, reactive astrocytes play important roles in repairing the blood-spinal cord barrier and restricting the size of the primary injury (Herrmann et al., J. Neurosci., 28:7231-7243 (2008); and Okada et al., Nature Med., 12:829-834 (2006)). However, in the sub-acute or chronic phase, reactive astrocytes constitute the major component of the glial scar, a dense tissue structure that is inhibitory to axonal regeneration (Silver and Miller, Nat. Rev. Neurosci., 5:146-156 (2004)). Therefore, for decades, substantial effort has been made to overcome the glial scar and promote regrowth of severed axons through the injury site (Filous and Schwab, Am. J. Pathol., 188(1):53-62 (2017)). On the other hand, the spinal neurons that are lost during and after the injury need to be replaced in order to rebuild the local neuronal circuits. In this regard, stem cell transplantation has been reported to achieve certain success (Lu et al., J. Clin. Inv., 127:3287-3299 (2017); and Tuszynski et al., Cell, 156:388-389 (2014)), however long-term survival of transplanted cells in the injury site has been an unsolved issue (Goldman, Cell Stem Cell, 18:174-188 (2016)). Thus, there remains a substantial unmet need for treating spinal cord injury.

Amyotrophic lateral sclerosis (ALS) is a late-onset fatal neurodegenerative disease affecting motor neurons with an incidence of about 1/100,000. Most ALS cases are sporadic, but 5-10% of the cases are familial ALS. Both sporadic and familial ALS (FALS) are associated with degeneration of cortical and spinal motor neurons. The etiology of ALS remains unknown. However, mutations of superoxide dismutase 1 (SOD1) have been known as the most common cause of FALS. Despite concentrated efforts in studying the underlying mechanisms causing disease pathology, there remains a substantial unmet need for treating ALS.

### SUMMARY

This document provides methods and materials involved in treating mammals having a spinal cord injury (SCI). For example, this document provides methods and materials for administering a composition containing exogenous nucleic acid encoding a NeuroD1 polypeptide (or a biologically active fragment thereof) alone or in combination with exogenous nucleic acid encoding a Dlx2 polypeptide (or a biologically active fragment thereof) to a mammal having SCI. This document also relates to methods and materials involved in treating mammals having amyotrophic lateral sclerosis (ALS). For example, this document provides methods and materials for administering a composition containing exogenous nucleic acid encoding a NeuroD1polypeptide (or a biologically active fragment thereof) to a mammal having ALS. This document also provides methods and materials for administering a composition containing exogenous nucleic acid encoding a NeuroD1 polypeptide (or a biologically active fragment thereof) alone or in combination with exogenous nucleic acid encoding an Isl1 polypeptide (or a biologically active fragment thereof) to a mammal having ALS.

In general, one aspect of this document features a method for treating a mammal having a spinal cord injury (SCI). The method comprises (or consists essentially of or consists of) administering a composition comprising (or consisting essentially of or consisting of) exogenous nucleic acid encoding a Neuronal Differentiation 1 (NeuroD1) polypeptide or a biologically active fragment thereof to the mammal. The mammal can be a human. The spinal cord injury can be due to a condition selected from the group consisting of: ischemic stroke; hemorrhagic stroke; physical injury; concussion; contusion; blast; penetration; tumor; inflammation; infection; traumatic spinal injury; ischemic or hemorrhagic myelopathy (spinal cord infarction); global ischemia as caused by cardiac arrest or severe hypotension (shock); hypoxic-ischemic encephalopathy as caused by hypoxia, hypoglycemia, or anemia; CNS embolism as caused by infective endocarditis or atrial myxoma; fibrocartilaginous embolic myelopathy; CNS thrombosis as caused by pediatric leukemia; cerebral venous sinus thrombosis as caused by nephrotic syndrome (kidney disease), chronic inflammatory disease, pregnancy, use of estrogen-based contraceptives, meningitis, dehydration; or a combination of any two or more thereof. The administering step can comprise delivering an expression vector comprising a nucleic acid encoding a NeuroD1 polypeptide or a biologically active fragment thereof to the spinal cord. The administering step can comprise delivering a recombinant viral expression vector comprising a nucleic acid encoding a NeuroD1 polypeptide or a biologically active fragment thereof to the spinal cord. The administering step can comprise delivering a recombinant adeno-associated virus expression vector comprising a nucleic acid encoding a NeuroD1 polypeptide or a biologically active fragment thereof to the spinal cord. The adeno-associated virus can be an AAV.PHP.eB. The administering step can comprise administering a recombinant expression vector comprising a nucleic acid sequence encoding NeuroD1 polypeptide, wherein the nucleic acid sequence encoding NeuroD1 polypeptide comprises a nucleic acid sequence selected from the group consisting of: a nucleic acid sequence encoding SEQ ID NO:2 or a functional fragment thereof; a nucleic acid sequence encoding SEQ ID NO:4 or a functional fragment thereof; SEQ ID NO:1 or a functional fragment thereof; SEQ ID NO:3 or a functional fragment thereof; and a nucleic acid sequence encoding a protein which has 70%, 71%, 72%, 73%, 74%, 75%, 76%, 77%, 78%, 79%, 81%, 82%, 83%, 84%, 85%, 86%, 87%, 88%, 89%, 90%, 91%, 92%, 93%, 94%, 95%, 96%, 97%, 98%, 99%, or greater, identity to SEQ ID NO: 2 or SEQ ID NO: 4, or a functional fragment thereof. The administering step can comprise a stereotactic injection to the spinal cord. The administering step can comprise an intravenous injection or intravenous infusion.

In another aspect, this document features a method of treating a mammal having a spinal cord injury. The method comprises (or consists essentially of or consists of) administering a pharmaceutical composition comprising (or consisting essentially of or consisting of) a pharmaceutically acceptable carrier containing adeno-associated virus particles comprising a nucleic acid encoding a NeuroD1 polypeptide or a biologically active fragment thereof to the spinal cord of the mammal. The pharmaceutical composition can comprise about 1 μL to about 500 μL of a pharmaceutically acceptable carrier containing adeno-associated virus at a concentration of 10¹⁰-10¹⁴ adeno-associated virus particles/mL of carrier comprising a nucleic acid encoding a NeuroD1 polypeptide or a biologically active fragment thereof. The pharmaceutical composition can be injected in the spinal cord of the mammal at a controlled flow rate of about 0.1 μL/minute to about 5 μL/minute.

In another aspect, this document features a method for treating a mammal having spinal cord injury. The method comprises (or consists essentially of or consists of) administering a composition comprising (or consisting essentially of or consisting of) exogenous nucleic acid encoding mir124, exogenous nucleic acid encoding a ISL LIM Homeobox 1 (Isl1) polypeptide or a biologically active fragment thereof, and exogenous nucleic acid encoding a LIM Homeobox 3 (Lhx3) polypeptide or biologically active fragment thereof to the spinal cord of the mammal. The mammal can be a human. The administering step can comprise delivering (i) an expression vector comprising a nucleic acid encoding mir124, (ii) an expression vector comprising a nucleic acid encoding a Isl1 polypeptide or a biologically active fragment thereof, and (iii) an expression vector comprising a nucleic acid encoding a polypeptide or biologically active fragment thereof Lhx3 to the spinal cord of the mammal. The administering step can comprise delivering (i) a recombinant viral expression vector comprising a nucleic acid encoding mir124, (ii) a recombinant viral expression vector comprising a nucleic acid encoding a Isl1 polypeptide or biologically active fragment thereof, and (iii) a recombinant viral expression vector comprising a nucleic acid encoding a Lhx3 polypeptide or a biologically active fragment thereof to the spinal cord of the mammal. The administering step can comprise delivering (i) a recombinant adeno-associated virus expression vector comprising a nucleic acid encoding mir124, (ii) a recombinant adeno-associated virus expression vector comprising a nucleic acid encoding a Isl1 polypeptide or a biologically active fragment thereof, and (iii) a recombinant adeno-associated virus expression vector comprising a nucleic acid encoding a Lhx3 polypeptide or biologically active fragment thereof to the spinal cord of the mammal. The administering step can comprise delivering an expression vector comprising a nucleic acid encoding mir124, a Isl1 polypeptide or a biologically active fragment thereof, and a Lhx3 polypeptide or a biologically active fragment thereof to the spinal cord of the mammal. The administering step can comprise delivering a recombinant viral expression vector comprising a nucleic acid encoding mir124, a Isl1 polypeptide or a biologically active fragment thereof, and a Lhx3 polypeptide or a biologically active fragment thereof to the spinal cord of the mammal. The administering step can comprise delivering a recombinant adeno-associated virus expression vector comprising a nucleic acid encoding mir124, a Isl1 polypeptide or biologically active fragment thereof, and a Lhx3 polypeptide or a biologically active fragment thereof to the spinal cord of the mammal. The administering step can further comprise administering therapeutically effective doses of one or more of exogenous nucleic acid encoding a Neurogenin 2 (Ngn2) polypeptide or a biologically active fragment thereof, mir218, and a nucleic acid encoding a NeuroD1 polypeptide or a biologically active fragment thereof in combination with any combination of mir124, a Isl1 polypeptide or a biologically active fragment thereof, or a Lhx3 polypeptide or a biologically active fragment thereof to the spinal cord of the mammal. The adeno-associated virus can be an AAV.PHP.eB.

In another aspect, this document features a method for treating a mammal having Amyotrophic lateral sclerosis (ALS). The method comprises (or consists essentially of or consists of) administering a composition comprising (or consisting essentially of or consisting of) exogenous nucleic acid encoding a NeuroD1 polypeptide or a biologically active fragment thereof to the central nervous system of the mammal. The mammal can be a human. The administering step can comprise delivering an expression vector comprising a nucleic acid encoding a NeuroD1 polypeptide or a biologically active fragment thereof to the brain. The administering step can comprise delivering a recombinant viral expression vector comprising a nucleic acid encoding a NeuroD1 polypeptide or a biologically active fragment thereof to the brain. The administering step can comprise delivering a recombinant adeno-associated virus expression vector comprising a nucleic acid encoding a NeuroD1 polypeptide or a biologically active fragment thereof to the brain. The adeno-associated virus can be an AAV.PHP.eB. The administering step can comprise administering a recombinant expression vector comprising a nucleic acid sequence encoding NeuroD1 protein, wherein the nucleic acid sequence encoding NeuroD1 protein comprises a nucleic acid sequence selected from the group consisting of: a nucleic acid sequence encoding SEQ ID NO:2 or a functional fragment thereof; a nucleic acid sequence encoding SEQ ID NO:4 or a functional fragment thereof; SEQ ID NO:1 or a functional fragment thereof; SEQ ID NO:3 or a functional fragment thereof; and a nucleic acid sequence encoding a protein which has 70%, 71%, 72%, 73%, 74%, 75%, 76%, 77%, 78%, 79%, 81%, 82%, 83%, 84%, 85%, 86%, 87%, 88%, 89%, 90%, 91%, 92%, 93%, 94%, 95%, 96%, 97%, 98%, 99%, or greater, identity to SEQ ID NO: 2 or SEQ ID NO: 4, or a functional fragment thereof. The administering step can comprise a stereotactic intracranial injection. The administering step can comprise two or more stereotactic intracranial injections. The administering step can comprise a retro-orbital injection.

In another aspect, this document features a method of treating a mammal having ALS. The method comprises (or consists essentially of or consists of) administering a pharmaceutical composition comprising (or consisting essentially of or consisting of) a pharmaceutically acceptable carrier containing adeno-associated virus particles comprising a nucleic acid encoding NeuroD1 to the central nervous system of the mammal. The pharmaceutical composition can comprise about 1 μL to about 500 μL of a pharmaceutically acceptable carrier containing adeno-associated virus at a concentration of 10¹⁰-10¹⁴ adeno-associated virus particles/ml of carrier comprising a nucleic acid encoding a NeuroD1 polypeptide. The pharmaceutical composition can be injected in the central nervous system of the mammal at a controlled flow rate of about 0.1 μL/minute to about 5 μL/minute.

In another aspect, this document features a method for treating a mammal having Amyotrophic lateral sclerosis (ALS). The method comprises (or consists essentially of or consists of) administering a composition comprising (or consisting essentially of or consisting of) exogenous nucleic acid encoding a NeuroD1 polypeptide or a biologically active fragment thereof, exogenous nucleic acid encoding an Isl1 polypeptide or a biologically active fragment thereof, and exogenous nucleic acid encoding a Lhx3 polypeptide or a biologically active fragment thereof to the central nervous system of the mammal. The mammal can be a human. The administering step can comprise delivering (i) an expression vector comprising a nucleic acid encoding a NeuroD1 polypeptide or a biologically active fragment thereof, (ii) an expression vector comprising a nucleic acid encoding a Isl1 polypeptide or a biologically active fragment thereof, and (iii) an expression vector comprising a nucleic acid encoding a Lhx3 polypeptide or a biologically active fragment thereof to the central nervous system of the mammal. The administering step can comprise delivering (i) a recombinant viral expression vector comprising a nucleic acid encoding a NeuroD1 polypeptide or a biologically active fragment thereof, (ii) a recombinant viral expression vector comprising a nucleic acid encoding a Isl1 polypeptide or a biologically active fragment thereof, and (iii) a recombinant viral expression vector comprising a nucleic acid encoding a Lhx3 polypeptide or a biologically active fragment thereof to the central nervous system of the mammal. The administering step can comprise delivering (i) a recombinant adeno-associated virus expression vector comprising a nucleic acid encoding a NeuroD1 polypeptide or a biologically active fragment thereof, (ii) a recombinant adeno-associated virus expression vector comprising a nucleic acid encoding a Isl1 polypeptide or a biologically active fragment thereof, and (iii) a recombinant adeno-associated virus expression vector comprising a nucleic acid encoding a Lhx3 polypeptide or a biologically active fragment thereof to the central nervous system of the mammal. The administering step can comprise delivering an expression vector comprising a nucleic acid encoding a NeuroD1 polypeptide or a biologically active fragment thereof, a Isl1 polypeptide or a biologically active fragment thereof and a Lhx3 polypeptide or a biologically active fragment thereof to the central nervous system of the mammal. The administering step can comprise delivering a recombinant viral expression vector comprising a nucleic acid encoding a NeuroD1 polypeptide or a biologically active fragment thereof, a Isl1 polypeptide or a biologically active fragment thereof, and a Lhx3 polypeptide or a biologically active fragment thereof to the central nervous system of the mammal. The administering step can comprise delivering a recombinant adeno-associated virus expression vector comprising a nucleic acid encoding a NeuroD1 polypeptide or a biologically active fragment thereof, a Isl1 polypeptide or a biologically active fragment thereof, and a Lhx3 polypeptide or a biologically active fragment thereof to the central nervous system of the mammal. The administering step can further comprise administering therapeutically effective doses of one or more of exogenous nucleic acid encoding Ngn2, mir218, and mir124 in combination with any combination of a NeuroD1 polypeptide or a biologically active fragment thereof, a Isl1 polypeptide or a biologically active fragment thereof, and a Lhx3 polypeptide or a biologically active fragment thereof to the central nervous system of the mammal. The adeno-associated virus can be an AAV.PHP.eB.

In another aspect, this document features a method for (1) regenerating dorsal spinal cord neurons, (2) generating new glutamatergic neurons, or (3) increasing circulation in the spinal cord within a mammal having a SCI and in need of the (1), (2), or (3). The method comprises (or consists essentially of or consists of) administering a composition comprising (or consisting essentially of or consisting of) exogenous nucleic acid encoding a NeuroD1 polypeptide or a biologically active fragment thereof to the mammal, wherein (a) the spinal cord neurons are regenerated, (b) new glutamatergic neurons are generated, or (c) spinal cord circulation is increased. The mammal can be a human. The administering step can comprise delivering an expression vector comprising a nucleic acid encoding a NeuroD1 polypeptide or a biologically active fragment thereof to the spinal cord. The administering step can comprise delivering a recombinant viral expression vector comprising a nucleic acid encoding a NeuroD1 polypeptide or a biologically active fragment thereof to the spinal cord. The administering step can comprise delivering a recombinant adeno-associated virus expression vector comprising a nucleic acid encoding a NeuroD1 polypeptide or a biologically active fragment thereof to the spinal cord. The adeno-associated virus can be an AAV.PHP.eB. The administering step can comprise administering a recombinant expression vector comprising a nucleic acid sequence encoding NeuroD1 polypeptide, wherein the nucleic acid sequence encoding NeuroD1 polypeptide comprises a nucleic acid sequence selected from the group consisting of: a nucleic acid sequence encoding SEQ ID NO:2 or a functional fragment thereof; a nucleic acid sequence encoding SEQ ID NO:4 or a functional fragment thereof; SEQ ID NO:1 or a functional fragment thereof; SEQ ID NO:3 or a functional fragment thereof; and a nucleic acid sequence encoding a protein which has 70%, 71%, 72%, 73%, 74%, 75%, 76%, 77%, 78%, 79%, 81%, 82%, 83%, 84%, 85%, 86%, 87%, 88%, 89%, 90%, 91%, 92%, 93%, 94%, 95%, 96%, 97%, 98%, 99%, or greater, identity to SEQ ID NO: 2 or SEQ ID NO: 4, or a functional fragment thereof. The administering step can comprise a stereotactic injection to the spinal cord. The administering step can comprise an intravenous injection or intravenous infusion.

In another aspect, this document features a method for (1) generating motor neurons, (2) reducing the number of microglia, or (3) reducing the number of reactive astrocytes within a mammal having ALS disease and in need of the (1), (2), or (3). The method comprises (or consists essentially of or consists of) administering a composition comprising (or consisting essentially of or consisting of) exogenous nucleic acid encoding a NeuroD1 polypeptide or a biologically active fragment thereof to the mammal, wherein (a) the motor neurons are generated, (b) the number of microglia is reduced, or (c) the number of reactive astrocytes is reduced. The mammal can be a human. The administering step can comprise delivering an expression vector comprising a nucleic acid encoding a NeuroD1 polypeptide or a biologically active fragment thereof to the spinal cord. The administering step can comprise delivering a recombinant viral expression vector comprising a nucleic acid encoding a NeuroD1 polypeptide or a biologically active fragment thereof to the spinal cord. The administering step can comprise delivering a recombinant adeno-associated virus expression vector comprising a nucleic acid encoding a NeuroD1 polypeptide or a biologically active fragment thereof to the spinal cord. The adeno-associated virus can be an AAV.PHP.eB. The administering step can comprise administering a recombinant expression vector comprising a nucleic acid sequence encoding NeuroD1 polypeptide, wherein the nucleic acid sequence encoding NeuroD1 polypeptide comprises a nucleic acid sequence selected from the group consisting of: a nucleic acid sequence encoding SEQ ID NO:2 or a functional fragment thereof; a nucleic acid sequence encoding SEQ ID NO:4 or a functional fragment thereof; SEQ ID NO:1 or a functional fragment thereof; SEQ ID NO:3 or a functional fragment thereof; and a nucleic acid sequence encoding a protein which has 70%, 71%, 72%, 73%, 74%, 75%, 76%, 77%, 78%, 79%, 81%, 82%, 83%, 84%, 85%, 86%, 87%, 88%, 89%, 90%, 91%, 92%, 93%, 94%, 95%, 96%, 97%, 98%, 99%, or greater, identity to SEQ ID NO: 2 or SEQ ID NO: 4, or a functional fragment thereof. The administering step can comprise a stereotactic injection to the spinal cord. The administering step can comprise an intravenous injection or intravenous infusion.

In another aspect, this document features a method for (1) regenerating dorsal spinal cord neurons, (2) generating new glutamatergic neurons, or (3) increasing circulation in the spinal cord within a mammal having a SCI and in need of the (1), (2), or (3). The method comprises (or consists essentially of or consists of) administering a composition comprising (or consisting essentially of or consisting of) exogenous nucleic acid encoding a NeuroD1 polypeptide or a biologically active fragment thereof, exogenous nucleic acid encoding a Isl1 polypeptide or a biologically active fragment thereof, or exogenous nucleic acid encoding a Lhx3 polypeptide or a biologically active fragment thereof to the mammal, wherein (a) the spinal cord neurons are regenerated, (b) new glutamatergic neurons are generated, or (c) spinal cord circulation is increased. The mammal can be a human. The administering step can comprise delivering (i) an expression vector comprising a nucleic acid encoding a NeuroD1 polypeptide or a biologically active fragment thereof, (ii) an expression vector comprising a nucleic acid encoding a Isl1 polypeptide or a biologically active fragment thereof, or (iii) an expression vector comprising a nucleic acid encoding a Lhx3 polypeptide or a biologically active fragment thereof to the central nervous system of the mammal. The administering step can comprise delivering (i) a recombinant viral expression vector comprising a nucleic acid encoding a NeuroD1 polypeptide or a biologically active fragment thereof, (ii) a recombinant viral expression vector comprising a nucleic acid encoding a Isl1 polypeptide or a biologically active fragment thereof, or (iii) a recombinant viral expression vector comprising a nucleic acid encoding a Lhx3 polypeptide or a biologically active fragment thereof to the central nervous system of the mammal. The administering step can comprise delivering (i) a recombinant adeno-associated virus expression vector comprising a nucleic acid encoding a NeuroD1 polypeptide or a biologically active fragment thereof, (ii) a recombinant adeno-associated virus expression vector comprising a nucleic acid encoding a Isl1 polypeptide or a biologically active fragment thereof, or (iii) a recombinant adeno-associated virus expression vector comprising a nucleic acid encoding a Lhx3 polypeptide or a biologically active fragment thereof to the central nervous system of the mammal. The adeno-associated virus can be an AAV.PHP.eB. The administering step can comprise a stereotactic injection to the spinal cord. The administering step can comprise an intravenous injection or intravenous infusion.

In another aspect, this document features a method for treating a mammal having spinal cord injury. The method comprises (or consists essentially of or consists of) administering a composition comprising (or consisting essentially of or consisting of) (a) exogenous nucleic acid encoding a NeuroD1 polypeptide or a biologically active fragment thereof and (b) exogenous nucleic acid encoding a Distal-Less Homeobox 2 (Dlx2) polypeptide or biologically active fragment thereof to the spinal cord of the mammal. The mammal can be a human. The administering step can comprise delivering (i) an expression vector comprising a nucleic acid a NeuroD1 polypeptide and (ii) an expression vector comprising a nucleic acid encoding a Dlx2 polypeptide or a biologically active fragment thereof to the spinal cord of the mammal. The administering step can comprise delivering (i) a recombinant viral expression vector comprising a nucleic acid encoding a NeuroD1 polypeptide and (ii) a recombinant viral expression vector comprising a nucleic acid encoding a Dlx2 polypeptide or biologically active fragment thereof to the spinal cord of the mammal. The administering step can comprise delivering (i) a recombinant adeno-associated virus expression vector comprising a nucleic acid encoding a NeuroD1 polypeptide and (ii) a recombinant adeno-associated virus expression vector comprising a nucleic acid encoding a Dlx2 polypeptide or a biologically active fragment thereof to the spinal cord of the mammal. The administering step can comprise delivering an expression vector comprising a nucleic acid encoding a NeuroD1 polypeptide or a biologically active fragment thereof and a Dlx2 polypeptide or a biologically active fragment thereof to the spinal cord of the mammal. The administering step can comprise delivering a recombinant viral expression vector comprising a nucleic acid encoding a NeuroD1 polypeptide or a biologically active fragment thereof and a Dlx2 polypeptide or a biologically active fragment thereof to the spinal cord of the mammal. The administering step can comprise delivering a recombinant adeno-associated virus expression vector comprising a nucleic acid encoding a NeuroD1 polypeptide or biologically active fragment thereof and a Dlx2 polypeptide or a biologically active fragment thereof to the spinal cord of the mammal. The adeno-associated virus can be an AAV.PHP.eB. The administering step can comprise administering a recombinant expression vector comprising a nucleic acid sequence encoding NeuroD1 polypeptide, wherein the nucleic acid sequence encoding NeuroD1 polypeptide comprises a nucleic acid sequence selected from the group consisting of: a nucleic acid sequence encoding SEQ ID NO:2 or a functional fragment thereof; a nucleic acid sequence encoding SEQ ID NO:4 or a functional fragment thereof; SEQ ID NO:1 or a functional fragment thereof; SEQ ID NO:3 or a functional fragment thereof; and a nucleic acid sequence encoding a protein which has 70%, 71%, 72%, 73%, 74%, 75%, 76%, 77%, 78%, 79%, 81%, 82%, 83%, 84%, 85%, 86%, 87%, 88%, 89%, 90%, 91%, 92%, 93%, 94%, 95%, 96%, 97%, 98%, 99%, or greater, identity to SEQ ID NO: 2 or SEQ ID NO: 4, or a functional fragment thereof. The administering step can comprise administering a recombinant expression vector comprising a nucleic acid sequence encoding Dlx2 polypeptide, wherein the nucleic acid sequence encoding Dlx2 polypeptide comprises a nucleic acid sequence selected from the group consisting of: a nucleic acid sequence encoding SEQ ID NO: 11 or a functional fragment thereof; a nucleic acid sequence encoding SEQ ID NO:13 or a functional fragment thereof; SEQ ID NO:10 or a functional fragment thereof; SEQ ID NO:12 or a functional fragment thereof; and a nucleic acid sequence encoding a protein which has 70%, 71%, 72%, 73%, 74%, 75%, 76%, 77%, 78%, 79%, 81%, 82%, 83%, 84%, 85%, 86%, 87%, 88%, 89%, 90%, 91%, 92%, 93%, 94%, 95%, 96%, 97%, 98%, 99%, or greater, identity to SEQ ID NO: 11 or SEQ ID NO: 13, or a functional fragment thereof. The administering step can comprise a stereotactic injection to the spinal cord. The administering step can comprise an intravenous injection or intravenous infusion. The adeno-associated virus can be an AAV serotype 5.

In another aspect, this document features a method for (1) regenerating dorsal spinal cord neurons, (2) generating new neurons, or (3) increasing circulation in the spinal cord within a mammal having a SCI and in need of the (1), (2), or (3). The method comprises (or consists essentially of or consists of) administering a composition comprising (or consisting essentially of or consisting of) (i) exogenous nucleic acid encoding a NeuroD1 polypeptide or a biologically active fragment thereof and (ii) exogenous nucleic acid encoding a Dlx2 polypeptide or a biologically active fragment thereof, wherein (a) the spinal cord neurons are regenerated, (b) new neurons are generated, or (c) spinal cord circulation is increased. The mammal can be a human. The administering step can comprise delivering (i) an expression vector comprising a nucleic acid encoding a NeuroD1 polypeptide or a biologically active fragment thereof and (ii) an expression vector comprising a nucleic acid encoding a Dlx2 polypeptide or a biologically active fragment thereof to the central nervous system of the mammal. The administering step can comprise delivering (i) a recombinant viral expression vector comprising a nucleic acid encoding a NeuroD1 polypeptide or a biologically active fragment thereof and (ii) a recombinant viral expression vector comprising a nucleic acid encoding a Dlx2 polypeptide or a biologically active fragment thereof to the central nervous system of the mammal. The administering step can comprise delivering (i) a recombinant adeno-associated virus expression vector comprising a nucleic acid encoding a NeuroD1 polypeptide or a biologically active fragment thereof and (ii) a recombinant adeno-associated virus expression vector comprising a nucleic acid encoding a Dlx2 polypeptide or a biologically active fragment thereof to the central nervous system of the mammal. The administering step can comprise delivering an expression vector comprising a nucleic acid encoding a NeuroD1 polypeptide or a biologically active fragment thereof and a Dlx2 polypeptide or a biologically active fragment thereof to the spinal cord of the mammal. The administering step can comprise delivering a recombinant viral expression vector comprising a nucleic acid encoding a NeuroD1 polypeptide or a biologically active fragment thereof and a Dlx2 polypeptide or a biologically active fragment thereof to the spinal cord of the mammal. The administering step can comprise delivering a recombinant adeno-associated virus expression vector comprising a nucleic acid encoding a NeuroD1 polypeptide or biologically active fragment thereof and a Dlx2 polypeptide or a biologically active fragment thereof to the spinal cord of the mammal. The adeno-associated virus can be an AAV.PHP.eB. The administering step can comprise a stereotactic injection to the spinal cord. The administering step can comprise an intravenous injection or intravenous infusion. The new neurons can be selected from the group consisting of glutamatergic neurons and GABAergic neurons. The new neurons can be glutamatergic neurons. The new neurons can be GABAergic neurons. The adeno-associated virus can be an AAV serotype 5.

In another aspect, this document features a method for treating a mammal having ALS. The method comprises (or consists essentially of or consists of) administering a composition comprising (a) exogenous nucleic acid encoding a NeuroD1 polypeptide or a biologically active fragment thereof and (b) exogenous nucleic acid encoding an Isl1 polypeptide or biologically active fragment thereof to the mammal. The mammal can be a human. The administering step can comprise delivering (i) an expression vector comprising a nucleic acid a NeuroD1 polypeptide and (ii) an expression vector comprising a nucleic acid encoding an Isl1 polypeptide or a biologically active fragment thereof to the mammal. The administering step can comprise delivering (i) a recombinant viral expression vector comprising a nucleic acid encoding a NeuroD1 polypeptide and (ii) a recombinant viral expression vector comprising a nucleic acid encoding an Isl1 polypeptide or biologically active fragment thereof to the mammal. The administering step can comprise delivering (i) a recombinant adeno-associated virus expression vector comprising a nucleic acid encoding a NeuroD1 polypeptide and (ii) a recombinant adeno-associated virus expression vector comprising a nucleic acid encoding an Isl1 polypeptide or a biologically active fragment thereof to the spinal cord of the mammal. The administering step can comprise delivering an expression vector comprising a nucleic acid encoding a NeuroD1 polypeptide or a biologically active fragment thereof and an Isl1 polypeptide or a biologically active fragment thereof to the mammal. The administering step can comprise delivering a recombinant viral expression vector comprising a nucleic acid encoding a NeuroD1 polypeptide or a biologically active fragment thereof and an Isl1 polypeptide or a biologically active fragment thereof to the mammal. The administering step can comprise delivering a recombinant adeno-associated virus expression vector comprising a nucleic acid encoding a NeuroD1 polypeptide or biologically active fragment thereof and an Isl1 polypeptide or a biologically active fragment thereof to the mammal. The adeno-associated virus can be an AAV.PHP.eB. The administering step can comprise administering a recombinant expression vector comprising a nucleic acid sequence encoding NeuroD1 polypeptide, wherein the nucleic acid sequence encoding NeuroD1 polypeptide comprises a nucleic acid sequence selected from the group consisting of: a nucleic acid sequence encoding SEQ ID NO:2 or a functional fragment thereof; a nucleic acid sequence encoding SEQ ID NO:4 or a functional fragment thereof; SEQ ID NO:1 or a functional fragment thereof; SEQ ID NO:3 or a functional fragment thereof; and a nucleic acid sequence encoding a protein which has 70%, 71%, 72%, 73%, 74%, 75%, 76%, 77%, 78%, 79%, 81%, 82%, 83%, 84%, 85%, 86%, 87%, 88%, 89%, 90%, 91%, 92%, 93%, 94%, 95%, 96%, 97%, 98%, 99%, or greater, identity to SEQ ID NO: 2 or SEQ ID NO: 4, or a functional fragment thereof. The administering step can comprise administering a recombinant expression vector comprising a nucleic acid sequence encoding an Isl1 polypeptide, wherein the nucleic acid sequence encoding an Isl1 polypeptide comprises a nucleic acid sequence selected from the group consisting of: a nucleic acid sequence encoding SEQ ID NO: 15 or a functional fragment thereof; a nucleic acid sequence encoding SEQ ID NO:17 or a functional fragment thereof; SEQ ID NO:14 or a functional fragment thereof; SEQ ID NO:16 or a functional fragment thereof; and a nucleic acid sequence encoding a protein which has 70%, 71%, 72%, 73%, 74%, 75%, 76%, 77%, 78%, 79%, 81%, 82%, 83%, 84%, 85%, 86%, 87%, 88%, 89%, 90%, 91%, 92%, 93%, 94%, 95%, 96%, 97%, 98%, 99%, or greater, identity to SEQ ID NO: 15 or SEQ ID NO: 17, or a functional fragment thereof.

In another aspect, this document features a method for treating a mammal having spinal cord injury. The method comprises (or consists essentially of or consists of) administering a composition comprising (a) exogenous nucleic acid encoding a NeuroD1 polypeptide or a biologically active fragment thereof and (b) exogenous nucleic acid encoding an Isl1 polypeptide or biologically active fragment thereof to the spinal cord of the mammal. The mammal can be a human. The administering step can comprise delivering (i) an expression vector comprising a nucleic acid a NeuroD1 polypeptide and (ii) an expression vector comprising a nucleic acid encoding an Isl1 polypeptide or a biologically active fragment thereof to the spinal cord of the mammal. The administering step can comprise delivering (i) a recombinant viral expression vector comprising a nucleic acid encoding a NeuroD1 polypeptide and (ii) a recombinant viral expression vector comprising a nucleic acid encoding an Isl1 polypeptide or biologically active fragment thereof to the spinal cord of the mammal. The administering step can comprise delivering (i) a recombinant adeno-associated virus expression vector comprising a nucleic acid encoding a NeuroD1 polypeptide and (ii) a recombinant adeno-associated virus expression vector comprising a nucleic acid encoding an Isl1 polypeptide or a biologically active fragment thereof to the spinal cord of the mammal. The administering step can comprise delivering an expression vector comprising a nucleic acid encoding a NeuroD1 polypeptide or a biologically active fragment thereof and an Isl1 polypeptide or a biologically active fragment thereof to the spinal cord of the mammal. The administering step can comprise delivering a recombinant viral expression vector comprising a nucleic acid encoding a NeuroD1 polypeptide or a biologically active fragment thereof and an Isl1 polypeptide or a biologically active fragment thereof to the spinal cord of the mammal. The administering step can comprise delivering a recombinant adeno-associated virus expression vector comprising a nucleic acid encoding a NeuroD1 polypeptide or biologically active fragment thereof and an Isl1 polypeptide or a biologically active fragment thereof to the spinal cord of the mammal. The adeno-associated virus can be an AAV.PHP.eB. The administering step can comprise administering a recombinant expression vector comprising a nucleic acid sequence encoding NeuroD1 polypeptide, wherein the nucleic acid sequence encoding NeuroD1 polypeptide comprises a nucleic acid sequence selected from the group consisting of: a nucleic acid sequence encoding SEQ ID NO:2 or a functional fragment thereof; a nucleic acid sequence encoding SEQ ID NO:4 or a functional fragment thereof; SEQ ID NO:1 or a functional fragment thereof; SEQ ID NO:3 or a functional fragment thereof; and a nucleic acid sequence encoding a protein which has 70%, 71%, 72%, 73%, 74%, 75%, 76%, 77%, 78%, 79%, 81%, 82%, 83%, 84%, 85%, 86%, 87%, 88%, 89%, 90%, 91%, 92%, 93%, 94%, 95%, 96%, 97%, 98%, 99%, or greater, identity to SEQ ID NO: 2 or SEQ ID NO: 4, or a functional fragment thereof. The administering step can comprise administering a recombinant expression vector comprising a nucleic acid sequence encoding an Isl1 polypeptide, wherein the nucleic acid sequence encoding an Isl1 polypeptide comprises a nucleic acid sequence selected from the group consisting of: a nucleic acid sequence encoding SEQ ID NO: 15 or a functional fragment thereof; a nucleic acid sequence encoding SEQ ID NO:17 or a functional fragment thereof; SEQ ID NO:14 or a functional fragment thereof; SEQ ID NO:16 or a functional fragment thereof; and a nucleic acid sequence encoding a protein which has 70%, 71%, 72%, 73%, 74%, 75%, 76%, 77%, 78%, 79%, 81%, 82%, 83%, 84%, 85%, 86%, 87%, 88%, 89%, 90%, 91%, 92%, 93%, 94%, 95%, 96%, 97%, 98%, 99%, or greater, identity to SEQ ID NO: 15 or SEQ ID NO: 17, or a functional fragment thereof.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1F. Neuronal conversion using NeuroD1-expressing retrovirus after stab injury in the spinal cord dorsal horn. (FIG. 1A) Experiment paradigm. (FIG. 1B) Schematic of dorsal horn injury and injection. Coordinates are 0.4 mm lateral of central artery and 0.4 mm below the tissue surface. Stab injury was performed with a 32-guage needle followed by stereotaxic injection at the injury site. Scale bar 500 μm. (FIG. 1C) Three main types of proliferating cells at 1 week post viral injection (wpi) after injection of Retro GFP: astrocytes, OPCs, and microglia. GFAP, Olig2, and Iba1 staining show markers for these cell types. Arrows show examples of each. Scale bar 50 μm. (FIG. 1D) Quantification based on staining for Retro GFP, 1 wpi samples. Bars show the mean and standard deviation of three replicates. (FIG. 1E) Converting cells in the dorsal horn 1 wpi, 3 wpi, and 6 wpi after Retro ND1-GFP injection. Maturing cells gradually up-regulate NeuN, adapt neuronal morphology, and reorganize. Arrows show example NeuN+ cells. Scale bar 500 μm. (FIG. 1F) Quantification based on staining for Retro ND1-GFP, 1W samples. Bars show the mean and standard deviation of three replicates.

FIGS. 2A-2F. Neuronal conversion using NeuroD1-expressing AAV9 after stab injury (e.g., a partial injury of the spinal cord) in the spinal cord dorsal horn. (FIG. 2A) Experiment paradigm. (FIG. 2B) AAV9 FLEX-NeuroD1-mCherry/AAV9 GFAP::Cre system (abbreviated elsewhere as AAV9 ND1-mCh). GFAP promoter restricts infected cells to astrocytes. Control virus replaces the ND1 transgene with an additional mCh. (FIG. 2C) Infected astrocytes in the dorsal horn 4 wpi after AAV9 mCh injection. Arrows and inset (2×mag) show an example GFAP+ cell. Scale bar 50 μm. (FIG. 2D) Converted neurons in the dorsal horn 4 wpi after AAV9-ND1-mChy injection. Arrows and inset (2×mag) show an example NeuN+ cell. Scale bar 50 μm. (FIG. 2E) Converting cells in the dorsal horn 2 wpi after AAV9-ND1-mChy injection. Arrows and inset (4×mag) show an example NeuN+/GFAP+ cell. Scale bar 50 am. (FIG. 2F) Quantification based on staining for AAV9 ND1-mCh, 2 wpi and 4 wpi, and AAV9 ND1-GFP or AAV9 GFP (Control), 8 wpi samples. Bars show the mean and standard deviation of three replicates. Infected cells at 2 wpi are mostly transitional, staining positive for NeuN and GFAP, and by 4 wpi are mostly converted, staining positive for only NeuN.

FIGS. 3A-3D. Subtypes of NeuroD1-converted neurons in the spinal cord dorsal horn. (FIG. 3A) Tlx3 (glutamatergic) and Pax2 (GABAergic) subtype staining for converted neurons in the dorsal horn 8 wpi after AAV9 ND1-GFP injection. Z-projection targets an example Tlx3+ neuron. Scale bar 50 μm. (FIG. 3B) Tlx3 and Pax2 subtype staining for converted neurons in the dorsal horn 6 wpi after Retro ND1-GFP injection. Z-projection targets an example Pax2+ neuron. Scale bar 50 μm. (FIG. 3C) 24 Quantification based on subtype staining for AAV9 ND1-GFP, 8 wpi and Retro ND1-GFP, 6 wpi samples. Control data is based on NeuN+ cells in uninjured, untreated tissue. Bars show the mean and standard deviation of three replicates. (FIG. 3D) AAV9 ND1-mCy and CaMK2-GFP coinjection at 4 wpi with strong (89.5±5.2%) co-labeling of CaMK2 for converted, Tlx3+ cells. Z-projection targets an example Tlx3+, CaMK2+ neuron. Scale bar 50 μm.

FIGS. 4A-4D. Region-specific subtypes of NeuroD1-converted neurons in the brain versus the spinal cord. (FIG. 4A) Subtype staining for converted neurons in the cortex 4 wpi after AAV9 ND1-mCh injection. Arrows show examples of cells positive for each subtype. Scale bar 50 μm. (FIG. 4B) 25 Quantification based on subtype staining in the cortex for AAV9 ND1-mCh, 4 wpi samples. Bars show the mean and standard deviation of three replicates. (FIG. 4C) Subtype staining for converted neurons in the spinal cord dorsal horn 4 wpi after AAV9 ND1-mChy injection. Arrows show examples of cells positive for each subtype. Scale bar 50 μm. (FIG. 4D) Quantification based on subtype staining in the spinal cord for AAV9 ND1-mCh, 4 wpi samples. Bars show the mean and standard deviation of three replicates.

FIGS. 5A-5I. Maturation and functionality of NeuroD1-converted neurons in the spinal cord dorsal horn. (FIG. 5A) Fluorescent/transmitted light image of a patch-clamped converted neuron. (FIG. 5B) Sample action potentials of a converted neuron. (FIG. 5C) Sample Na+ and K+ currents of a converted neuron. (FIG. 5D) Sample EPSCs of a converted neuron. (FIG. 5E) Na+ current amplitudes for converted and native neurons. (FIG. 5F) EPSC amplitudes and frequencies for converted and native neurons. (FIG. 5G) Synaptic SV2 and VGluT1 puncta for converted neurons in the dorsal horn 8 wpi after AAV9 ND1-GFP injection. Arrows and inset (4×mag) show an example cell with puncta visible on its soma and processes. Scale bar 50 am. (FIG. 5H) Synaptic SV2 and VGluT2 puncta for converted neurons in the dorsal horn 8 wpi after AAV9 ND1-GFP injection. Arrows and inset (4×mag) show an example cell with puncta visible on its soma and processes. Scale bar 50 am. (FIG. 5I) Integration of converted neurons into local network in the dorsal horn 8 wpi after AAV9 ND1-GFP injection. Activated neurons indicated by cFos staining are a subset of all neurons.

FIGS. 6A-6F. NeuroD1 converts reactive astrocytes into neurons around the injury core with a short delay of viral injection after contusive SCI. (FIG. 6A) AAV9 FLEX expressing either GFP reporter alone or NeuroD1-GFP were injected along with AAV9 GFAP::Cre to target reactive astrocytes at 10 days after a contusive SCI (30 Kdyn force). Spinal cords were analyzed at 6 wpi. (FIG. 6B) 28 Experiment paradigm. (FIG. 6C) Many infected cells survived around the injury core (indicated by *) and showing distinct cellular morphology between the two groups. Immunostaining of the neuronal markers GFAP and NeuN indicates successful neuronal conversion from reactive astrocytes by ND1-GFP. Scale bars 50 μm at low-mag, 20 μm at high-mag. (FIG. 6D) Estimated number of converted neurons per infection (average number of NeuN+ infected cells per horizontal section, calculated from one dorsal, one central, and one ventral section, multiplied by the total number of horizontal sections per sample (n=3 for each group; *, p<0.01). (FIG. 6E) NeuN acquisition at 6 wpi (n=3 for each group; *, p<0.01). (FIG. 6F) GFAP loss at 6 wpi (n=3 for each group; *, p<0.01).

FIGS. 7A-7I. NeuroD1-mediated neuronal conversion with a long delay of viral injection after contusive SCI. (FIG. 7A) AAV9 FLEX expressing either GFP reporter alone or NeuroD1-GFP were injected along with AAV9 GFAP::Cre to target reactive astrocytes at 16 weeks after a contusive SCI (30 Kdyn force). The spinal cords were analyzed at 10 wpi. (FIG. 7B) Infected cells survived around the injury core (indicated by *) and showing distinct cellular morphology between the 30 two groups. (FIG. 7C) Co-expression of the astrocyte marker S100b in control GFP+ cells. Scale bar 50 μm. (FIG. 7D) Immunostaining of the neuronal marker NeuN indicates successful neuronal conversion from reactive astrocytes by ND1-GFP with high efficiency. Scale bar 50 μm. (FIG. 7E) NeuN acquisition at 10 wpi (n=4 for each group; ***, p<0.005). (FIG. 7F) Co-expression of ND1 protein in the ND1-GFP+ cells with a typical neuronal morphology. Scale bar 20 μm. (FIG. 7G) Co-expression of the mature neuronal marker SV2 in the ND1-GFP+ cells. (FIG. 7H) Co-expression of the neuronal activity marker cFos in the ND1-GFP+ cells. Scale bar 20 μm. (FIG. 7I) Co-expression of the glutamatergic subtype marker Tlx3 in ND1-GFP+ neurons in the spinal cord dorsal horn. Arrows show an example Tlx3+ cell. Scale bar 20 μm.

FIGS. 8A-8B. Infected cells by AAV9-ND1-mChy overexpress ND1 protein in the injured spinal cord. (FIG. 8A) Immunostaining analysis confirmed that the infected cells by AAV9 ND1-mCh expressed the neuronal marker NeuN indicating neuronal conversion in the dorsal horn of injured spinal cord at 4 wpi. Scale bar 200 μm. (FIG. 8B) Infected cells overexpressed ND1 protein at 4 wpi. Scale bar 50 μm.

FIG. 9. NeuroD1-mediated neuronal conversion does not involve apoptosis. TUNEL assays were performed to detect apoptotic cells at different stages of neuronal conversion by AAV9 ND1-mCh in the injured spinal cord. Arrows show infected cells that are NeuN+ but TUNEL-.

FIGS. 10A-10B. CaMK2-GFP virus and GAD-GFP mice can be used to confirm neuronal subtype. (FIG. 10A) CaMK2-GFP (from co-injected AAV9 virus) co-stains with Tlx3 while (FIG. 10B) GAD-GFP (from GAD-GFP transgenic mouse) co-stains with Pax2, indicating that these markers can be used to confirm glutamatergic and GABAergic subtypes in the dorsal horn. Scale bar 200 μm. Dotted boxes at 4×mag below.

FIGS. 11A-11D. (FIG. 11A) Schematic of stab injury and viral infection sites. (FIG. 11B) Schematic of constructs in infections. (FIG. 11C) Staining following infection shows co-stains for RFP, NeuN and GFAP. (FIG. 11D) Panel of markers following different combinations of infections.

FIGS. 12A-12E. (FIG. 12A) Panel of markers following infection with control (mCherry) or MIL (mir124, Isl1, and Lhx3) at 1 week post infection (wpi) and 3 weeks post infection (wpi). (FIG. 12B) Histogram of NeuN⁺RFP⁺/RFP⁺ for mCherry and MIL at 1 wpi. (FIG. 12C) Histogram of NeuN⁺RFP⁺/RFP⁺ for mCherry and MIL at 3 wpi. (FIG. 12D) Histogram of GFAP⁺RFP⁺/RFP⁺ for mCherry and MIL at 1 wpi. (FIG. 12E) Histogram of GFAP⁺RFP⁺/RFP⁺ for mCherry and MIL at 3 wpi.

FIGS. 13A-13G. NeuN immunostaining for mir124, Isl1, and Lhx3. Both Isl1 and Lhx3 can efficiently convert astrocytes into neurons. * indicates p<0.05.

FIGS. 14A-14C. Immunostaining with motor neuron marker, ChAT, found that Isl1 can convert astrocytes into motoneurons.

FIGS. 15A-15C. ChAT staining also indicates that Lhx3 can convert astrocytes into motor neurons. **P<0.01.

FIG. 16. These results confirmed that GFAP::Cre expression of Cre in astrocytes, but not in converted neurons.

FIGS. 17A-17D. These results indicate that, in GFAP::Cre transgenic mice, NeuroD1 can also convert astrocytes into neurons.

FIGS. 18A-18C. ND1 expression increases the paw print area of SOD1-G93A mice in a catwalk assay at 20 weeks. Untreated n=5; EF1a-GFP n=7; and EF1a-ND1-GFP n=8. * indicates p<0.05.

FIG. 19. SOD1-G93A mice gait analysis by a catwalk assay at 20 weeks. Untreated n=5; EF1a-GFP n=7; and EF1a-ND1-GFP n=8.

FIG. 20. ND1 expression increases paw swing speed in SOD1-G93A mice at 20 weeks as assessed by a catwalk assay. *** indicates p<0.001.

FIG. 21. ND1 expression shortens step cycle paw swing speed in SOD1-G93A mice at 20 weeks as assessed by a catwalk assay. EF1a-GFP n=8; EF1a-ND1-GFP n=9. ** indicates p<0.01.

FIG. 22. ND1 expression increases mobility and leg movement in SOD1-G93A mice at 24 weeks. EF1a-GFP n=4; EF1a-ND1-GFP n=7. * indicates p<0.05.

FIGS. 23A-23C. Expression of GFP, NeuroD1, and SOD1 in spinal cord (FIG. 23A), dorsal horn (FIG. 23B), or ventral horn (FIG. 23C) for mice infected with the indicated virus.

FIGS. 24A-24F. Expression of GFP, CHAT (a motor neuron marker), Iba1, iNOS, and/or GFAP in spinal cord (FIG. 24A-FIG. 24C), ventral horn (FIG. 24D-FIG. 24E), or cerebra, cerebellum, and ventral horn (FIG. 24F) for mice infected with the indicated virus.

FIGS. 25A-25L. Expression analysis of SOD1 mice injected with ND1/Isl1/Lhx expressing viruses (DIL group) or control viruses (Con group) via retro-orbital injection. The DIL group received the following viruses: AAV.PHP.eB-GFAP-Cre (1.6×10¹⁰ genome copies) plus AAV.PHP.eB-Flex-ND1-GFP (1.9×10¹⁰ genome copies) plus AAV.PHP.eB-Flex-Isl1-mCherry (1.3×10¹⁰ genome copies) plus AAV.PHP.eB-Flex-Lhx31-mCherry (1.5×10¹⁰ genome copies). The Con group received the following viruses: AAV.PHP.eB-GFAP-Cre (1.8×10¹⁰ genome copies) plus AAV.PHP.eB-Flex-GFP (0.8×10¹⁰ genome copies) plus AAV.PHP.eB-Flex-mCherry (3.4×10¹⁰ genome copies). Viral injection at 9.4 wks old, and mice sacrificed at 22.1 wks. Male=1 mouse for Con Group and 2 mice for DIL group; female=1 mouse for Con Group and 2 mice for DIL group. Expression of GFP (left panels) and RFP (right panels) in cortex, brainstem, and thalamus of mice from the DIL group and control group. Both Control and DIL groups showed wide infection (FIG. 25A). Control group exhibited about 30-40% leakage in cortex and brainstem, and greater than 90% leakage in thalamus. mCherry exhibited a little more leakage than GFP. DIL group demonstrated that all neurons express GFP and RFP signals. Infected cells were more located in the ventral horn of the spinal cord (FIG. 25B). Cre signals were located in astrocytes with the Con group exhibiting much more Cre signal (FIG. 25C). In the DIL group, GFP+ cells expressed ND1 signal, and RFP⁺ cells expressed Isl1 signal (FIG. 25D). The intensity of Isl1 expression varied in different regions in the brain (FIG. 25E). The distribution of Tbr1 signals exhibited little difference between the Control (CON) and DIL groups (FIG. 25F). Iba1 signals had higher density and brightness in the Control (CON) group in brain (FIG. 25G) and in spinal cord (FIG. 25H). There were more motor neurons in the DIL group in cervical spinal cord, ventral horn (FIG. 25I). Neuron loss in the motor column was severe in both the CON and DIL groups in the lumbar (FIG. 25J). Motor neurons were hard to detect in the lumbar spinal cord, and neurodegeneration was too severe in the lumbar spinal cord at the end stage (22.1 weeks). The blood vessels did not show huge differences in high magnification images (FIG. 25K). The blood vessels had higher density and thickness in the DIL group (FIG. 25L).

FIGS. 26A-26D. (FIG. A) Staining for NeuroD1 (ND1) and Dlx2 at 4 wpi following infection with AAV5-ND1-mCherry (AAV5-ND1-mCh) and AAV5-Dlx2-mCherry (AAV5-Dlx2-mCh). (FIG. B) Panel of markers (Tlx3 and Pax2) at 4 wpi following co-infection with AAV5-ND1-mCh and AAV5-Dlx2-mCh. (FIG. C) Histogram of Tlx3⁺ cells and Pax2⁺ cells for AAV5-ND1 alone and AAV5-ND1-mCh+AAV5-Dlx2-mCh at 4 wpi. (FIG. D) Staining for Pax2 following infection in GAD-GFP mice at 4 wpi with AAV5-ND1-mCh+AAV5-Dlx2-mCh.

FIGS. 27A-27B. NeuroD1-Mediated Astrocyte-to-Neuron Conversion in ALS mice. (FIG. 27A) Injecting control AAV expressing mCherry (RFP staining) into spinal cord revealed infection of GFAP-positive reactive astrocytes in the ventral horn of ALS mice. (FIG. 27B) AAV NeuroD1-mCherry infected cells were immunopositive for NeuroD1 (ND1), NeuN (neuronal marker), and ChAT (motor neuron marker). Some NeuroD1-converted neurons were ChAT-positive motor neurons in the spinal cord ventral horn. Scale bar, 50 μm.

FIGS. 28A-28B. Intrathecal injection of viruses designed to express ND1+Isl1 and viruses designed to express ND1+Lhx3 converts astrocytes into neurons in the ventral horn of ALS mice. (FIG. 28A) Following intrathecal injection, AAV PHP.eB-GFAP::Cre plus AAV PHP.eB-Flex-GFP specifically targeted astrocytes in the ventral horn, as shown by left column GFP control group. However, in both ND1-GFP+Isl1-mCherry (middle column) and ND1-GFP+Lhx3-mCherry (right column) groups, GFP-positive cells were converted into neurons in the ventral horn of ALS mice. There were more converted neurons in ND1+Isl1 group. Scale bar: 200 μm. Viral injection was at 9 week old ALS mice, and immunostaining was at 21 week old mice. (FIG. 28B) Immunostaining of NeuN, ND1, Isl1, GFP, and RFP in NeuroD1+Isl1 group. Scale bar: 40 μm.

FIGS. 29A-29B. Quantification of motor neurons after gene therapy treatment in different segments of the spinal cord in ALS mice. (FIG. 29A) Immunostaining with a motor neuron-specific marker, ChAT, revealed the motor neuron number in wild type (WT) mice and mice from the ND1+Isl1, ND1+Lhx3, and GFP control groups. A substantial reduction of motor neurons was observed in the GFP control group. Scale bar: 100 μm. (FIG. 29B) Quantitative analyses of motor neurons in different segments of the spinal cord of WT, ND1+Isl1, ND1+Lhx3, and GFP control groups.

FIGS. 30A-30B. Viral delivery of NeuroD1+Isl1 reduces microglia-mediated inflammation in ALS mice. (FIG. 30A) GFAP immunostaining revealed repressed astroglial activation in the cervical cord treated by viruses designed to express ND1+Isl1. Scale bar: 40 m. (FIG. 30B) Repression of microglial activation was observed by decreased Iba1 and CD11b immunostaining in the ND1+Isl1 group in ALS mice. Scale bar: 100 μm.

FIGS. 31A-31G. Partial rescue of body weight and mobility after delivery of viruses designed to express NeuroD1+Isl1. (FIG. 31A) The ND1+Isl1 treatment partially rescued the body weight in ALS mice. (FIG. 31B) The ND1+Lhx3-treated ALS mice exhibited higher mortality rate after intrathecal AAV injection, although motor neuron number was increased. (FIG. 31C) Leg stretching measurement at the week 22. (FIG. 31D-31F) Quantified results on leg stretching experiments: the average distance of leg stretching (FIG. 31D), the number of leg stretching (FIG. 31E), and the total mobility time (FIG. 31F). The ALS mice treated with viruses designed to express ND1+Isl1 exhibited better motor function. (FIG. 31G) The ALS mice treated with viruses designed to express ND1+Isl1 exhibited longer duration in the hanging wire test.

FIGS. 32A-32C. Open field test showing moderate improvement after gene therapy treatment. (A-B) In the open field test, the ALS mice treated with viruses designed to express ND1+Isl1 exhibited increased total distance traveled in the open field box (FIG. 32A) and increased mobile time (FIG. 32B), compared with untreated ALS mice and ALS mice treated with viruses designed to express ND1+Lhx3. (FIG. 32C) Typical traces of the mouse movement in the open field box.

FIGS. 33A-C. Catwalk gait analysis showed improvement of motor functions in ALS mice treated with viruses designed to express ND1+Isl1. (FIG. 33A) Illustration of catwalk foot print of the ALS mice among different groups. (FIG. 33B-33C) Quantified data showing that ALS mice treated with viruses designed to express ND1+Isl1 displayed increased paw print area in either the contralateral (FIG. 33B) or the ipsilateral (FIG. 33C) comparison.

### DETAILED DESCRIPTION

This document provides methods and materials for treating mammals having a SCI. For example, this document provides methods and materials for administering a composition containing exogenous nucleic acid encoding a NeuroD1polypeptide to a mammal having a SCI. In another example, this document provides methods and materials for administering a composition containing exogenous nucleic acid encoding a NeuroD1 polypeptide and a Dlx2 polypeptide to a mammal having a SCI. In another example, this document provides methods and materials for administering a composition containing exogenous nucleic acid encoding a mir124 microRNA, exogenous nucleic acid encoding an Isl1 polypeptide, and/or exogenous nucleic acid encoding an Lhx3 polypeptide to a mammal having a SCI. In another example, this document provides methods and materials for administering a composition containing exogenous nucleic acid encoding NeuroD1, exogenous nucleic acid encoding a mir124 microRNA, exogenous nucleic acid encoding an Isl1 polypeptide, and/or exogenous nucleic acid encoding an Lhx3 polypeptide to a mammal having a SCI.

This document also provides methods and materials involved in treating mammals having ALS. For example, this document provides methods and materials for administering a composition containing exogenous nucleic acid encoding a NeuroD1 polypeptide to a mammal having ALS. In another example, this document provides methods and materials for administering a composition containing exogenous nucleic acid encoding a NeuroD1 polypeptide, exogenous nucleic acid encoding an Isl1 polypeptide, and exogenous nucleic acid encoding a Lhx3 polypeptide to a mammal having ALS. In another example, this document provides methods and materials for administering a composition containing exogenous nucleic acid encoding a NeuroD1 polypeptide and exogenous nucleic acid encoding an Isl1 polypeptide to a mammal having ALS.

Any appropriate mammal can be identified as having a neurological disorder (e.g., ALS) in the brain and/or central nervous system. For example, humans and other primates such as monkeys can be identified as having ALS. Thus, humans, non-human primates, cats, dogs, sheep, goats, horses, cows, pigs and rodents (e.g., mice and rats) having a neurological disorder (e.g., ALS) in the brain and/or central nervous system can be treated as described herein. Any appropriate mammal can be identified as having a spinal cord injury. For example, humans and other primates such as monkeys can be identified as having a spinal cord injury. Thus, humans, non-human primates, cats, dogs, sheep, goats, horses, cows, pigs and rodents (e.g., mice and rats) having a spinal cord injury can be treated as described herein

In some cases, administration of a therapeutically effective amount of (i) exogenous nucleic acid encoding any of the polypeptides, or any combination of polypeptides, described herein (e.g., NeuroD1, Isl1, Lhx3, and Ngn2) and (ii) exogenous nucleic acid encoding mir124 and mir218 to a subject affected by a neurological disorder (e.g., ALS) in the brain mediates: the generation of new glutamatergic neurons by conversion of reactive astrocytes to glutamatergic neurons; reduction of the number of reactive astrocytes; survival of injured neurons including GABAergic and glutamatergic neurons; the generation of new non-reactive astrocytes; the reduction of reactivity of non-converted reactive astrocytes; reintegration of blood vessels into the injured region; the generation of motor neurons, the reduction of the number of microglia, and the reduction of the number of reactive astrocyte.

In some cases, administration of a therapeutically effective amount of (i) exogenous nucleic acid encoding any of the polypeptides, or any combination of polypeptides, described herein (e.g., NeuroD1, Isl1, Lhx3, Dlx2, and Ngn2) and/or (ii) exogenous nucleic acid encoding mir124 and mir218 to a subject affected by a spinal cord injury mediates: the generation of new glutamatergic neurons by conversion of reactive astrocytes to glutamatergic neurons; reduction of the number of reactive astrocytes; survival of injured neurons including GABAergic and glutamatergic neurons; the generation of new non-reactive astrocytes; the reduction of reactivity of non-converted reactive astrocytes; reintegration of blood vessels into the injured region, and regenerating dorsal spinal cord neurons.

In some cases, a method or composition provided herein regenerates dorsal spinal cord neurons, increasing the number of dorsal spinal cord neurons from a baseline level by between about 1% and 100% after administration of a composition provided herein. In some cases, a method or composition provided herein generates regenerates dorsal spinal cord neurons, increasing the number of dorsal spinal cord neurons from a baseline level by between about 1% and about 10%, between 1% and about 20%, between 1% and about 30%, between 10% and about 20%, between 10% and about 30%, between about 10% and about 40%, between about 20% and about 30%, between about 20% and about 40%, between about 20% and about 50%, between about 30% and about 40%, between about 30% and about 50%, between about 30% and about 60%, between about 40% and about 50%, between about 40% and about 60%, between about 40% and about 70%, between about 50% and about 60%, between about 50% and about 70%, between about 50% and about 80%, between about 60% and about 70%, between about 60% and about 80%, between about 60% and about 90%, between about 70% and about 80%, between about 70% and about 90%, between about 70% and about 100%, between about 80% and about 90%, between about 80% and about 100%, or between about 90% and about 100%.

In some cases, a method or composition provided herein generates new glutamatergic neurons, increasing the number of glutamatergic neurons from a baseline level by between about 1% and 500% after administration of a composition provided herein. In some cases, a method or composition provided herein generates new glutamatergic neurons, increasing the number of glutamatergic neurons from a baseline level by between about 1% and 50%, between about 1% and 100%, between about 1% and 150%, between about 50% and 100%, between about 50% and 150%, between about 50% and 200%, between about 100% and 150%, between about 100% and 200%, between 100% and 250%, between about 150% and 200%, between about 150% and 250%, between about 150% and 300%, between 200% and 250%, between 200% and 300%, between 200% and 350%, between 250% and 300%, between 250% and 350%, between about 250% and 400%, between about 300% and 350%, between about 300% and 400%, between about 300% and 450%, between about 350% and 400%, between about 350% and 450%, between about 350% and 500%, between about 400% and 450%, between about 400% and 500%, or between about 450% and 500% after administration of a composition provided herein.

In some cases, a method or composition provided herein increases circulation in the spinal cord between about 1% and 100% after administration of a composition provided herein. In some cases, a method or composition provided herein increases circulation in the spinal cord between about 1% and about 10%, between 1% and about 20%, between 1% and about 30%, between 10% and about 20%, between 10% and about 30%, between about 10% and about 40%, between about 20% and about 30%, between about 20% and about 40%, between about 20% and about 50%, between about 30% and about 40%, between about 30% and about 50%, between about 30% and about 60%, between about 40% and about 50%, between about 40% and about 60%, between about 40% and about 70%, between about 50% and about 60%, between about 50% and about 70%, between about 50% and about 80%, between about 60% and about 70%, between about 60% and about 80%, between about 60% and about 90%, between about 70% and about 80%, between about 70% and about 90%, between about 70% and about 100%, between about 80% and about 90%, between about 80% and about 100%, or between about 90% and about 100% after administration of a composition provided herein.

In some cases, a method or composition provided herein generates motor neurons, increasing the number of motor neurons from a baseline level by between about 1% and 500% after administration of a composition provided herein. In some cases, a method or composition provided herein generates motor neurons, increasing the number of motor neurons from a baseline level by between about 1% and 50%, between about 1% and 100%, between about 1% and 150%, between about 50% and 100%, between about 50% and 150%, between about 50% and 200%, between about 100% and 150%, between about 100% and 200%, between 100% and 250%, between about 150% and 200%, between about 150% and 250%, between about 150% and 300%, between 200% and 250%, between 200% and 300%, between 200% and 350%, between 250% and 300%, between 250% and 350%, between about 250% and 400%, between about 300% and 350%, between about 300% and 400%, between about 300% and 450%, between about 350% and 400%, between about 350% and 450%, between about 350% and 500%, between about 400% and 450%, between about 400% and 500%, or between about 450% and 500% after administration of a composition provided herein.

In some cases, a method or composition provided herein reduces microglia, reducing the number of microglia from a baseline level by between about 1% and 100% after administration of a composition provided herein. In some cases, a method or composition provided herein reduces the microglia, reducing the number of microglia from a baseline level by between by between about 1% and about 10%, between 1% and about 20%, between 1% and about 30%, between 10% and about 20%, between 10% and about 30%, between about 10% and about 40%, between about 20% and about 30%, between about 20% and about 40%, between about 20% and about 50%, between about 30% and about 40%, between about 30% and about 50%, between about 30% and about 60%, between about 40% and about 50%, between about 40% and about 60%, between about 40% and about 70%, between about 50% and about 60%, between about 50% and about 70%, between about 50% and about 80%, between about 60% and about 70%, between about 60% and about 80%, between about 60% and about 90%, between about 70% and about 80%, between about 70% and about 90%, between about 70% and about 100%, between about 80% and about 90%, between about 80% and about 100%, or between about 90% and about 100% after administration of a composition provided herein.

In some cases, a method or composition provided herein reduces the number of reactive astrocytes by between about 1% and 100% after administration of a composition provided herein. In some cases, a method or composition provided herein reduces the number of reactive astrocytes by between about 1% and about 10%, between 1% and about 20%, between 1% and about 30%, between 10% and about 20%, between 10% and about 30%, between about 10% and about 40%, between about 20% and about 30%, between about 20% and about 40%, between about 20% and about 50%, between about 30% and about 40%, between about 30% and about 50%, between about 30% and about 60%, between about 40% and about 50%, between about 40% and about 60%, between about 40% and about 70%, between about 50% and about 60%, between about 50% and about 70%, between about 50% and about 80%, between about 60% and about 70%, between about 60% and about 80%, between about 60% and about 90%, between about 70% and about 80%, between about 70% and about 90%, between about 70% and about 100%, between about 80% and about 90%, between about 80% and about 100%, or between about 90% and about 100% after administration of a composition provided herein.

In some cases, administration of a therapeutically effective amount of (i) exogenous nucleic acid encoding any of the polypeptides, or any combination of polypeptides, described herein (e.g., NeuroD1, Isl1, Lhx3, Dlx2, and Ngn2) and/or (ii) exogenous nucleic acid encoding mir124 and mir218 to a subject affected by a neurological disorder (e.g., ALS) in the brain or having a spinal cord injury mediates: reduced inflammation at the injury site; reduced neuroinhibition at the injury site; re-establishment of normal microglial morphology at the injury site; re-establishment of neural circuits at the injury site, increased blood vessels at the injury site; re-establishment of blood-brain-barrier at the injury site; re-establishment of normal tissue structure at the injury site; and improvement of motor deficits due to the disruption of normal blood flow.

In some cases, administration of a therapeutically effective amount of (i) exogenous nucleic acid encoding any of the polypeptides, or any combination of polypeptides, described herein (e.g., NeuroD1, Isl1, Lhx3, Dlx2, and Ngn2) and/or (ii) exogenous nucleic acid encoding mir124 and mir218 or any combination thereof to ameliorate the effects of a neurological disorder (e.g., ALS) in the brain in an individual subject in need thereof has greater beneficial effects when administered to reactive astrocytes than to quiescent astrocytes. In some cases, administration of a therapeutically effective amount of (i) exogenous nucleic acid encoding any of the polypeptides, or any combination of polypeptides, described herein (e.g., NeuroD1, Isl1, Lhx3, Dlx2, and Ngn2) and/or (ii) exogenous nucleic acid encoding mir124 and mir218 to ameliorate the effects of a spinal cord injury in an individual subject in need thereof has greater beneficial effects when administered to reactive astrocytes than to quiescent astrocytes.

In some cases, a method for treating a mammal having spinal cord injury can include administrating a therapeutically effective amount of a composition, expression vector, or adeno-associated expression vector including a nucleic acid sequence encoding a NeuroD1 polypeptide (or a biologically active fragment thereof) and a nucleic acid sequence encoding a Dlx2 polypeptide (or biologically active fragment thereof). In some cases, nucleic acid sequence encoding a NeuroD1 polypeptide (or a biologically active fragment thereof) and a nucleic acid sequence encoding a Dlx2 polypeptide (or biologically active fragment thereof) are subcloned into one expression vector. In some cases, nucleic acid sequence encoding a NeuroD1 polypeptide (or a biologically active fragment thereof) and a nucleic acid sequence encoding a Dlx2 polypeptide (or biologically active fragment thereof) are subcloned into separate expression vectors.

In some cases, a method for (1) regenerating dorsal spinal cord neurons, (2) generating new neurons, and/or (3) increasing circulation in the spinal cord within a mammal having a SCI and in need of said (1), (2), and/or (3) can include administering a composition including exogenous nucleic acid encoding a NeuroD1 polypeptide (or a biologically active fragment thereof) and exogenous nucleic acid encoding a Dlx2 polypeptide (or a biologically active fragment thereof) under conditions wherein (a) the spinal cord neurons are regenerated, (b) new neurons are generated, and/or (c) spinal cord circulation is increased. In some cases, new neurons are selected from the group consisting of glutamatergic neurons and GABAergic neurons. In some cases, new neurons are glutamatergic neurons. In some cases, new neurons are GABAergic neurons.

In some cases, a method for treating a mammal having spinal cord injury can include administrating a therapeutically effective amount of a composition, expression vector, or adeno-associated expression vector including a nucleic acid sequence encoding a NeuroD1 polypeptide (or a biologically active fragment thereof) and a nucleic acid sequence encoding an Isl1 polypeptide (or biologically active fragment thereof). In some cases, nucleic acid sequence encoding a NeuroD1 polypeptide (or a biologically active fragment thereof) and a nucleic acid sequence encoding an Isl1 polypeptide (or biologically active fragment thereof) are subcloned into one expression vector. In some cases, nucleic acid sequence encoding a NeuroD1 polypeptide (or a biologically active fragment thereof) and a nucleic acid sequence encoding an Isl1 polypeptide (or biologically active fragment thereof) are subcloned into separate expression vectors.

In some cases, a spinal cord injury can be due to a condition selected from the group consisting of ischemic stroke, hemorrhagic stroke, physical injury, concussion, contusion, blast, penetration, tumor, inflammation, infection, traumatic spinal injury, ischemic or hemorrhagic myelopathy (spinal cord infarction), global ischemia, hypoxic-ischemic encephalopathy, CNS embolism as caused by, fibrocartilaginous embolic myelopathy, CNS thrombosis, and cerebral venous sinus thrombosis.

In some cases, global ischemia is caused by cardiac arrest or severe hypotension (shock). In some cases, hypoxic-ischemic encephalopathy is caused by hypoxia, hypoglycemia, or anemia. In some cases, CNS embolism is caused by infective endocarditis or atrial myxoma. In some cases, CNS thrombosis is caused by pediatric leukemia. In some cases, cerebral venous sinus thrombosis is caused by nephrotic syndrome (kidney disease), chronic inflammatory disease, pregnancy, use of estrogen-based contraceptives, meningitis, or dehydration.

In some cases, a spinal cord injury is due to ischemic stroke. In some cases, a spinal cord injury is due to hemorrhagic stroke. In some cases, a spinal cord injury is due to a physical injury. In some cases, a spinal cord injury is due to concussion. In some cases, a spinal cord injury is due to contusion. In some cases, a spinal cord injury is due to a blast. In some cases, a spinal cord injury is due to penetration. In some cases, a spinal cord injury is due to a tumor. In some cases, a spinal cord injury is due to inflammation. In some cases, a spinal cord injury is due to infection. In some cases, a spinal cord injury is due traumatic spinal injury. In some cases, a spinal cord injury is due to ischemic or hemorrhagic myelopathy (spinal cord infaction). In some cases, a spinal cord injury is due to global ischemia. In some cases, a spinal cord injury is due to hypoxic-ischemic encephalopathy. In some cases, a spinal cord injury is due to CNS embolism. In some cases, a spinal cord injury is due to fibrocartilaginous embolic myelopathy. In some cases, a spinal cord injury is due to CNS thrombosis. In some cases, a spinal cord injury is due to cerebral venous sinus. In some cases, a spinal cord injury is due to thrombosis.

In some cases, a method for treating a mammal having ALS can include administrating a therapeutically effective amount of a composition, expression vector, or adeno-associated expression vector including a nucleic acid sequence encoding a NeuroD1 polypeptide (or a biologically active fragment thereof) and a nucleic acid sequence encoding an Isl1 polypeptide (or biologically active fragment thereof). In some cases, nucleic acid sequence encoding a NeuroD1 polypeptide (or a biologically active fragment thereof) and a nucleic acid sequence encoding an Isl1 polypeptide (or biologically active fragment thereof) are subcloned into one expression vector. In some cases, nucleic acid sequence encoding a NeuroD1 polypeptide (or a biologically active fragment thereof) and a nucleic acid sequence encoding an Isl1 polypeptide (or biologically active fragment thereof) are subcloned into separate expression vectors.

Treatment with (i) exogenous nucleic acid encoding any of the polypeptides, or any combination of polypeptides, described herein (e.g., NeuroD1, Isl1, Lhx3, Dlx2, and Ngn2) and/or (ii) exogenous nucleic acid encoding mir124 and mir218 can be administered to the region of injury as diagnosed by magnetic resonance imaging (MRI). Electrophysiology can assess functional changes in neural firing as caused by neural cell death or injury. Non-invasive methods to assay neural damage include EEG. Disruption of blood flow to a point of injury may be non-invasively assayed via Near Infrared Spectroscopy and functional magnetic resonance (fMRI). Blood flow within the region may either be increased, as seen in aneurysms, or decreased, as seen in ischemia. Injury to the CNS caused by disruption of blood flow additionally causes short-term and long-term changes to tissue structure that can be used to diagnose point of injury. In the short term, injury will cause localized swelling. In the long term, cell death will cause points of tissue loss. Non-invasive methods to assay structural changes caused by tissue death include MRI, position emission tomography (PET) scan, computerized axial tomography (CAT) scan, or ultrasound. These methods may be used singularly or in any combination to pinpoint the focus of injury.

As described above, non-invasive methods to assay structural changes caused by tissue death include MRI, CAT scan, or ultrasound. Functional assay may include EEG recording.

In some embodiments of the methods for treating a neurological disorder as described herein, (i) exogenous nucleic acid encoding any of the polypeptides, or any combination of polypeptides, described herein (e.g., NeuroD1, Isl1, Lhx3, Dlx2, and Ngn2) and/or (ii) exogenous nucleic acid encoding mir124 and mir218 is administered as an expression vector containing a nucleic acid sequence encoding any of the polypeptides described herein or mir218 and/or mir214.

In some embodiments of the methods for treating a neurological disorder as described herein, a viral vector (e.g., an AAV) including (i) exogenous nucleic acid encoding any of the polypeptides, or any combination of polypeptides, described herein (e.g., NeuroD1, Isl1, Lhx3, Dlx2, and Ngn2) and/or (ii) exogenous nucleic acid encoding mir124 and/or mir218 is delivered by injection into the brain of a subject, such as stereotaxic intracranial injection or retro-orbital injection. In some cases, the composition containing the adeno-associated virus including (i) exogenous nucleic acid encoding any of the polypeptides, or any combination of polypeptides, described herein (e.g., NeuroD1, Isl1, Lhx3, Dlx2, and Ngn2) and/or (ii) exogenous nucleic acid encoding mir124 and/or mir218 is administered to the brain using two more intracranial injections at the same location in the brain. In some cases, the composition containing the adeno-associated virus including (i) exogenous nucleic acid encoding any of the polypeptides, or any combination of polypeptides, described herein (e.g., NeuroD1, Isl1, Lhx3, Dlx2, and Ngn2) and/or (ii) exogenous nucleic acid encoding mir124 and/or mir218 is administered to the brain using two more intracranial injections at two or more different locations in the brain.

In some embodiments of the methods for treating a spinal cord injury as described herein, a viral vector (e.g., an AAV) (i) exogenous nucleic acid encoding any of the polypeptides, or any combination of polypeptides, described herein (e.g., NeuroD1, Isl1, Lhx3, Dlx2, and Ngn2) and/or (ii) exogenous nucleic acid encoding mir124 and/or mir218 is delivered by injection into spinal cord of a subject, such as stereotaxic injection, or by intravenous infusion or intravenous injection.

In some embodiments, the gene delivery vector can be an AAV vector. For example, an AAV vector can be selected from the group of: an AAV2 vector, an AAV5 vector, and an AAV8 vector, an AAV1 vector, an AAV7 vector, an AAV9 vector, an AAV3 vector, an AAV6 vector, an AAV10 vector, and an AAV11 vector.

The term “expression vector” refers to a recombinant vehicle for introducing (i) exogenous nucleic acid encoding any of the polypeptides, or any combination of polypeptides, described herein (e.g., NeuroD1, Isl1, Lhx3, Dlx2, and Ngn2) and/or (ii) exogenous nucleic acid encoding mir124 and mir218 into a host cell in vitro or in vivo where the nucleic acid is expressed to produce the polypeptide as described herein.

In particular embodiments, an expression vector including SEQ ID NO: 1 or 3 or a substantially identical nucleic acid sequence is expressed to produce NeuroD1 in cells containing the expression vector. In particular embodiments, an expression vector including SEQ ID NO: 10 or 12 or a substantially identical nucleic acid sequence is expressed to produce Dlx2 in cells containing the expression vector. The term “recombinant” is used to indicate a nucleic acid construct in which two or more nucleic acids are linked and which are not found linked in nature. Expression vectors include, but are not limited to plasmids, viruses, BACs and YACs. Particular viral expression vectors illustratively include those derived from adenovirus, adeno-associated virus, retrovirus, and lentivirus.

This document describes material and methods for treating a neurological disorder (e.g., ALS) or a spinal cord injury in a subject in need thereof according to the methods described which include providing a viral vector including (i) exogenous nucleic acid encoding any of the polypeptides, or any combination of polypeptides, described herein (e.g., NeuroD1, Isl1, Lhx3, Dlx2, and Ngn2) and/or (ii) exogenous nucleic acid encoding mir124 and mir218; and delivering the viral vector to the brain or spinal cord of the subject, whereby the viral vector infects glial cells of the central nervous system, respectively, producing infected glial cells and whereby the (i) exogenous nucleic acid encoding any of the polypeptides, or any combination of polypeptides, described herein (e.g., NeuroD1, Isl1, Lhx3, Dlx2, and Ngn2) and/or (ii) exogenous nucleic acid encoding mir124 and mir218 are expressed in the infected glial cells at a therapeutically effective level, wherein the expression of the polypeptides or miRNA, or a combination of polypeptides and miRNA, in the infected cells results in a greater number of neurons in the subject compared to an untreated subject having the same neurological condition, whereby the neurological disorder or spinal cord injury is treated. In addition to the generation of new neurons, the number of reactive glial cells will also be reduced, resulting in less neuroinhibitory factors released, less neuroinflammation, more blood vessels that are also evenly distributed, thereby making local environment more permissive to neuronal growth or axon penetration, hence alleviating neurological conditions.

In some cases, adeno-associated vectors can be used in a method described herein and will infect both dividing and non-dividing cells, at an injection site. Adeno-associated viruses (AAV) are ubiquitous, noncytopathic, replication-incompetent members of ssDNA animal virus of parvoviridae family. In some cases, any of various recombinant adeno-associated viruses, such as serotypes 1-11, can be used as described herein. In some cases, an AAV-PHP.eb is used to administer the exogenous NeuroD1. In some cases, an AAV-PHP.eb is used to administer the exogenous Dlx2. In some cases, an AAV-PHP.eb is used to administer the exogenous Isl1. In some cases, an AAV serotype 5 is used to administer the exogenous NeuroD1. In some cases, an AAV serotype 5 is used to administer the exogenous Dlx2. In some cases, an AAV serotype 5 is used to administer the exogenous Isl1.

A “FLEX” switch approach is used to express, for example, NeuroD1 in infected cells according to some aspects described herein. The terms “FLEX” and “flip-excision” are used interchangeably to indicate a method in which two pairs of heterotypic, antiparallel loxP-type recombination sites are disposed on either side of an inverted NeuroD1 coding sequence which first undergo an inversion of the coding sequence followed by excision of two sites, leading to one of each orthogonal recombination site oppositely oriented and incapable of further recombination, achieving stable inversion, see for example Schnutgen et al., Nature Biotechnology, 21:562-565 (2003); and Atasoy et al, J. Neurosci., 28:7025-7030 (2008). Since the site-specific recombinase under control of a glial cell-specific promoter will be strongly expressed in glial cells, including reactive astrocytes, NeuroD1 will also be expressed in glial cells, including reactive astrocytes. Then, when the stop codon in front of NeuroD1 is removed from recombination, the constitutive or neuron-specific promoter will drive high expression of NeuroD1, allowing reactive astrocytes to be converted into functional neurons. According to particular aspects, (i) exogenous nucleic acid encoding any of the polypeptides, or any combination of polypeptides, described herein (e.g., NeuroD1, Isl1, Lhx3, Dlx2, and Ngn2) and/or (ii) exogenous nucleic acid encoding mir124 and mir218 is administered to a subject in need thereof by administration of 1) an adeno-associated virus expression vector including a DNA sequence encoding a site-specific recombinase under transcriptional control of an astrocyte-specific promoter such as GFAP or S100b or AldhlL1; and 2) an adeno-associated virus expression vector including (i) exogenous nucleic acid encoding any of the polypeptides, or any combination of polypeptides, described herein (e.g., NeuroD1, Isl1, Lhx3, Dlx2, and Ngn2) and/or (ii) exogenous nucleic acid encoding mir124 and mir218 under transcriptional control of a ubiquitous (constitutive) promoter or a neuron-specific promoter wherein the nucleic acid sequence encoding the exogenous nucleic acid sequence is inverted and in the wrong orientation for expression of the polypeptides and miRNA until the site-specific recombinase inverts the inverted nucleic acid sequence encoding the polypeptides and/or miRNA, thereby allowing expression of the polypeptides and/or miRNA.

Site-specific recombinases and their recognition sites include, for example, Cre recombinase along with recognition sites loxP and lox2272 sites, or FLP-FRT recombination, or their combinations.

A composition including (i) exogenous nucleic acid encoding any of the polypeptides, or any combination of polypeptides, described herein (e.g., NeuroD1, Isl1, Lhx3, Dlx2, and Ngn2) and/or (ii) exogenous nucleic acid encoding mir124 and mir218 (e.g., an AAV including (i) exogenous nucleic acid encoding any of the polypeptides, or any combination of polypeptides, described herein (e.g., NeuroD1, Isl1, Lhx3, Dlx2, and Ngn2) and/or (ii) exogenous nucleic acid encoding mir124 and mir218 can be formulated into a pharmaceutical composition for administration into a mammal. For example, a therapeutically effective amount of the composition including an exogenous nucleic acid encoding a NeuroD1 polypeptide (e.g., an AAV encoding a NeuroD1 polypeptide) can be formulated with one or more pharmaceutically acceptable carriers (additives) and/or diluents. In some cases, a therapeutically effective amount of the composition including an exogenous nucleic acid encoding a Dlx2 polypeptide (e.g., an AAV encoding a Dlx2 polypeptide) can be formulated with one or more pharmaceutically acceptable carriers (additives) and/or diluents. A pharmaceutical composition including an exogenous nucleic acid encoding a NeuroD1 polypeptide (e.g., an AAV encoding a NeuroD1 polypeptide) can be formulated for various routes of administration, for example, for oral administration as a capsule, a liquid or the like. In some cases, a pharmaceutical composition including an exogenous nucleic acid encoding a Dlx2 polypeptide (e.g., an AAV encoding a Dlx2 polypeptide) can be formulated for various routes of administration, for example, for oral administration as a capsule, a liquid or the like. In some cases, a pharmaceutical composition including an exogenous nucleic acid encoding a Isl1 polypeptide (e.g., an AAV encoding a Isl1 polypeptide) can be formulated for various routes of administration, for example, for oral administration as a capsule, a liquid or the like. In some cases, a viral vector (e.g., AAV) including (i) exogenous nucleic acid encoding any of the polypeptides, or any combination of polypeptides, described herein (e.g., NeuroD1, Isl1, Lhx3, Dlx2, and Ngn2) and/or (ii) exogenous nucleic acid encoding mir124 and mir218 is administered parenterally, preferably by intravenous injection or intravenous infusion. The administration can be, for example, by intravenous infusion, for example for 60 minutes, for 30 minutes or for 15 minutes. In some cases, the administration can be between 1 minute and 60 minutes. In some cases, the administration can be between 1 minute and 5 minutes, between 1 minute and 10 minutes, between 1 minute and 15 minutes, between 5 minutes and 10 minutes, between 5 minutes and 15 minutes, between 5 minutes and 20 minutes, between 10 minutes and 15 minutes, between 10 minutes and 20 minutes, between 10 minutes and 25 minutes, between 15 minutes and 20 minutes, between 15 minutes and 25 minutes, between 15 minutes and 30 minutes, between 20 minutes and 25 minutes, between 20 minutes and 30 minutes, between 20 minutes and 35 minutes, between 25 minutes and 30 minutes, between 25 minutes and 35 minutes, between 25 minutes and 40 minutes, between 30 minutes and 35 minutes, between 30 minutes and 40 minutes, between 30 minutes and 45 minutes, between 35 minutes and 40 minutes, between 35 minutes and 45 minutes, between 35 minutes and 50 minutes, between 40 minutes and 45 minutes, between 40 minutes and 50 minutes, between 40 minutes and 55 minutes, between 45 minutes and 50 minutes, between 45 minutes and 55 minutes, between 45 minutes and 60 minutes, between 50 minutes and 55 minutes, between 50 minutes and 60 minutes, or between 55 minutes and 60 minutes. In some cases, the viral vector {e.g., AAV including (i) exogenous nucleic acid encoding any of the polypeptides, or any combination of polypeptides, described herein (e.g., NeuroD1, Isl1, Lhx3, Dlx2, and Ngn2) and/or (ii) exogenous nucleic acid encoding mir124 and mir218} is administered locally by injection to the brain during a surgery. Compositions which are suitable for administration by injection and/or infusion include solutions and dispersions, and powders from which corresponding solutions and dispersions can be prepared. Such compositions will comprise the viral vector and at least one suitable pharmaceutically acceptable carrier. Suitable pharmaceutically acceptable carriers for intravenous administration include, but not limited to, bacterostatic water, Ringer's solution, physiological saline, phosphate buffered saline (PBS) and Cremophor EL™. Sterile compositions for the injection and/or infusion can be prepared by introducing the viral vector {e.g., AAV including (i) exogenous nucleic acid encoding any of the polypeptides, or any combination of polypeptides, described herein (e.g., NeuroD1, Isl1, Lhx3, Dlx2, and Ngn2) and/or (ii) exogenous nucleic acid encoding mir124 and mir218} in the required amount into an appropriate carrier, and then sterilizing by filtration. Compositions for administration by injection or infusion should remain stable under storage conditions after their preparation over an extended period of time. The compositions can contain a preservative for this purpose. Suitable preservatives include, but not limited to, chlorobutanol, phenol, ascorbic acid and thimerosal.

A pharmaceutical composition can be formulated for administration in solid or liquid form including, without limitation, sterile solutions, suspensions, sustained-release formulations, tablets, capsules, pills, powders, and granules. The formulations can be presented in unit-dose or multi-dose containers, for example, sealed ampules and vials, and may be stored in a freeze dried (lyophilized) condition requiring only the addition of the sterile liquid carrier, for example, water for injections, immediately prior to use. Extemporaneous injection solutions and suspensions may be prepared from sterile powders, granules, and tablets.

Additional pharmaceutically acceptable carriers, fillers, and vehicles that may be used in a pharmaceutical composition described herein include, without limitation, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

As used herein, the term “adeno-associated virus particle” refers to packaged capsid forms of the AAV virus that transmits its nucleic acid genome to cells.

An effective amount of composition containing an (i) exogenous nucleic acid encoding any of the polypeptides, or any combination of polypeptides, described herein (e.g., NeuroD1, Isl1, Lhx3, Dlx2, and Ngn2) and/or (ii) exogenous nucleic acid encoding mir124 and mir218 can be any amount that ameliorates the symptoms of the neurological disorder within a mammal (e.g., a human) without producing severe toxicity to the mammal. For example, an effective amount of adeno-associated virus encoding a NeuroD1 polypeptide can be a concentration from about 10¹⁰ to 10¹⁴ adeno-associated virus particles/mL. In some cases, an effective amount of adeno-associated virus encoding a NeuroD1 polypeptide can be between 10¹⁰ adeno-associated virus particles/mL and 10¹¹ adeno-associated virus particles/mL, between 10¹⁰ adeno-associated virus particles/mL and 10¹² adeno-associated virus particles/mL, between 10¹⁰ adeno-associated virus particles/mL and 10¹³ adeno-associated virus particles/mL, between 10¹¹ adeno-associated virus particles/mL and 10¹² adeno-associated virus particles/mL, between 10¹¹ adeno-associated virus particles/mL and 10¹³ adeno-associated virus particles/mL, between 10¹¹ adeno-associated virus particles/mL and 10¹⁴ adeno-associated virus particles/mL, between 10¹² adeno-associated virus particles/mL and 10¹³ adeno-associated virus particles/mL, between 10¹² adeno-associated virus particles/mL and 10¹⁴ adeno-associated virus particles/mL, or between 10¹³ adeno-associated virus particles/mL and 10¹⁴ adeno-associated virus particles/mL. In some cases, an effective amount of adeno-associated virus encoding a Dlx2 polypeptide can be a concentration from about 10¹⁰ to 10¹⁴ adeno-associated virus particles/mL. In some cases, an effective amount of adeno-associated virus encoding a Dlx2 polypeptide can be between 10¹⁰ adeno-associated virus particles/mL and 10¹¹ adeno-associated virus particles/mL, between 10¹⁰ adeno-associated virus particles/mL and 10¹² adeno-associated virus particles/mL, between 10¹⁰ adeno-associated virus particles/mL and 10¹³ adeno-associated virus particles/mL, between 10¹¹ adeno-associated virus particles/mL and 10¹² adeno-associated virus particles/mL, between 10¹¹ adeno-associated virus particles/mL and 10¹³ adeno-associated virus particles/mL, between 10¹¹ adeno-associated virus particles/mL and 10¹⁴ adeno-associated virus particles/mL, between 10¹² adeno-associated virus particles/mL and 10¹³ adeno-associated virus particles/mL, between 10¹² adeno-associated virus particles/mL and 10¹⁴ adeno-associated virus particles/mL, or between 10¹³ adeno-associated virus particles/mL and 10¹⁴ adeno-associated virus particles/mL. In some cases, an effective amount of adeno-associated virus encoding an Isl1 polypeptide can be a concentration from about 10¹⁰ to 10¹⁴ adeno-associated virus particles/mL. In some cases, an effective amount of adeno-associated virus encoding an Isl1 polypeptide can be between 10¹⁰ adeno-associated virus particles/mL and 10¹¹ adeno-associated virus particles/mL, between 10¹⁰ adeno-associated virus particles/mL and 10¹² adeno-associated virus particles/mL, between 10¹⁰ adeno-associated virus particles/mL and 10¹³ adeno-associated virus particles/mL, between 10¹¹ adeno-associated virus particles/mL and 10¹² adeno-associated virus particles/mL, between 10¹¹ adeno-associated virus particles/mL and 10¹³ adeno-associated virus particles/mL, between 10¹¹ adeno-associated virus particles/mL and 10¹⁴ adeno-associated virus particles/mL, between 10¹² adeno-associated virus particles/mL and 10¹³ adeno-associated virus particles/mL, between 10¹² adeno-associated virus particles/mL and 10¹⁴ adeno-associated virus particles/mL, or between 10¹³ adeno-associated virus particles/mL and 10¹⁴ adeno-associated virus particles/mL. If a particular mammal fails to respond to a particular amount, then the amount of the AAV encoding a NeuroD1 polypeptide can be increased. In some case, if a particular mammal fails to respond to a particular amount, then the amount of the AAV encoding a polypeptide can be increased. Factors that are relevant to the amount of viral vector (e.g., an AAV encoding an exogenous nucleic acid encoding a NeuroD1 polypeptide) to be administered are, for example, the route of administration of the viral vector, the nature and severity of the disease, the disease history of the patient being treated, and the age, weight, height, and health of the patient to be treated. In some cases, the expression level of the polypeptides or miRNA as described herein, which is required to achieve a therapeutic effect, the immune response of the patient, as well as the stability of the gene product are relevant for the amount to be administered. In some cases, the administration of the viral vector (e.g., an AAV encoding a NeuroD1 polypeptide) occurs in an amount which leads to a complete or substantially complete healing of the dysfunction or disease of the brain or spinal cord.

In some cases, an effective amount of composition containing (i) exogenous nucleic acid encoding any of the polypeptides, or any combination of polypeptides, described herein (e.g., NeuroD1, Isl1, Lhx3, Dlx2, and Ngn2) and/or (ii) exogenous nucleic acid encoding mir124 and mir218 can be any administered at a controlled flow rate of about 0.1 μL/minute to about 5 μL/minute.

In some cases, the controlled flow rate is between 0.1 μL/minute and 0.2 μL/minute, between 0.1 μL/minute and 0.3 μL/minute, between 0.1 μL/minute and 0.4 μL/minute, between 0.2 μL/minute and 0.3 μL/minute, between 0.2 μL/minute and 0.4 μL/minute, between 0.2 μL/minute and 0.5 μL/minute, between 0.3 μL/minute and 0.4 μL/minute, between 0.3 μL/minute and 0.5 μL/minute, between 0.3 μL/minute and 0.6 μL/minute, between 0.4 μL/minute and 0.5 μL/minute, between 0.4 μL/minute and 0.6 μL/minute, between 0.4 μL/minute and 0.7 μL/minute, between 0.5 μL/minute and 0.6 μL/minute, between 0.5 μL/minute and 0.7 μL/minute, between 0.5 μL/minute and 0.8 μL/minute, between 0.6 μL/minute and 0.7 μL/minute, between 0.6 μL/minute and 0.8 μL/minute, between 0.6 μL/minute and 0.9 μL/minute, between 0.7 μL/minute and 0.8 μL/minute, between 0.7 μL/minute and 0.9 μL/minute, between 0.7 μL/minute and 1.0 μL/minute, between 0.8 μL/minute and 0.9 μL/minute, between 0.8 μL/minute and 1.0 μL/minute, between 0.8 μL/minute and 1.1 μL/minute, between 0.9 μL/minute and 1.0 μL/minute, between 0.9 μL/minute and 1.1 μL/minute, between 0.9 μL/minute and 1.2 μL/minute, between 1.0 μL/minute and 1.1 μL/minute, between 1.0 μL/minute and 1.2 μL/minute, between 1.0 μL/minute and 1.3 μL/minute, between 1.1 μL/minute and 1.2 μL/minute, between 1.1 μL/minute and 1.3 μL/minute, between 1.1 μL/minute and 1.4 μL/minute, between 1.2 μL/minute and 1.3 μL/minute, between 1.2 μL/minute and 1.4 μL/minute, between 1.2 μL/minute and 1.5 μL/minute, between 1.3 μL/minute and 1.4 μL/minute, between 1.3 μL/minute and 1.5 μL/minute, between 1.3 μL/minute and 1.6 μL/minute, between 1.4 μL/minute and 1.5 μL/minute, between 1.4 μL/minute and 1.6 μL/minute, between 1.4 μL/minute and 1.7 μL/minute, between 1.5 μL/minute and 1.6 μL/minute, between 1.5 μL/minute and 1.7 μL/minute, between 1.5 μL/minute and 1.8 μL/minute, between 1.6 μL/minute and 1.7 μL/minute, between 1.6 μL/minute and 1.8 μL/minute, between 1.6 μL/minute and 1.9 μL/minute, between 1.7 μL/minute and 1.8 μL/minute, between 1.7 μL/minute and 1.9 μL/minute, between 1.7 μL/minute and 2.0 μL/minute, between 1.8 μL/minute and 1.9 μL/minute, between 1.8 μL/minute and 2.0 μL/minute, between 1.8 μL/minute and 2.1 μL/minute, between 1.9 μL/minute and 2.0 μL/minute, between 1.9 μL/minute and 2.1 μL/minute, between 1.9 μL/minute and 2.2 μL/minute, between 2.0 μL/minute and 2.1 μL/minute, between 2.0 μL/minute and 2.2 μL/minute, between 2.0 μL/minute and 2.3 μL/minute, between 2.1 μL/minute and 2.2 μL/minute, between 2.1 μL/minute and 2.3 μL/minute, between 2.1 μL/minute and 2.4 μL/minute, between 2.2 μL/minute and 2.3 μL/minute, between 2.2 μL/minute and 2.4 μL/minute, between 2.2 μL/minute and 2.5 μL/minute, between 2.3 μL/minute and 2.4 μL/minute, between 2.3 μL/minute and 2.5 μL/minute, between 2.3 μL/minute and 2.6 μL/minute, between 2.4 μL/minute and 2.5 μL/minute, between 2.4 μL/minute and 2.6 μL/minute, between 2.4 μL/minute and 2.7 μL/minute, between 2.5 μL/minute and 2.6 μL/minute, between 2.5 μL/minute and 2.7 μL/minute, between 2.5 μL/minute and 2.8 μL/minute, between 2.6 μL/minute and 2.7 μL/minute, between 2.6 μL/minute and 2.8 μL/minute, between 2.6 μL/minute and 2.9 μL/minute, between 2.7 μL/minute and 2.8 μL/minute, between 2.7 μL/minute and 2.9 μL/minute, between 2.7 μL/minute and 3.0 μL/minute, between 2.8 μL/minute and 2.9 μL/minute, between 2.8 μL/minute and 3.0 μL/minute, between 2.8 μL/minute and 3.1 μL/minute, between 2.9 μL/minute and 3.0 μL/minute, between 2.9 μL/minute and 3.1 μL/minute, between 2.9 μL/minute and 3.2 μL/minute, between 3.0 μL/minute and 3.1 μL/minute, between 3.0 μL/minute and 3.2 μL/minute, between 3.0 μL/minute and 3.3 μL/minute, between 3.1 μL/minute and 3.2 μL/minute, between 3.1 μL/minute and 3.3 μL/minute, between 3.1 μL/minute and 3.4 μL/minute, between 3.2 μL/minute and 3.3 μL/minute, between 3.2 μL/minute and 3.4 μL/minute, between 3.2 μL/minute and 3.5 μL/minute, between 3.3 μL/minute and 3.4 μL/minute, between 3.3 μL/minute and 3.5 μL/minute, between 3.3 μL/minute and 3.6 μL/minute, between 3.4 μL/minute and 3.5 μL/minute, between 3.4 μL/minute and 3.6 μL/minute, between 3.4 μL/minute and 3.7 μL/minute, between 3.5 μL/minute and 3.6 μL/minute, between 3.5 μL/minute and 3.7 μL/minute, between 3.5 μL/minute and 3.8 μL/minute, between 3.6 μL/minute and 3.7 μL/minute, between 3.6 μL/minute and 3.8 μL/minute, between 3.6 μL/minute and 3.9 μL/minute, between 3.7 μL/minute and 3.8 μL/minute, between 3.7 μL/minute and 3.9 μL/minute, between 3.7 μL/minute and 4.0 μL/minute, between 3.8 μL/minute and 3.9 μL/minute, between 3.8 μL/minute and 4.0 μL/minute, between 3.8 μL/minute and 4.1 μL/minute, between 3.9 μL/minute and 4.0 μL/minute, between 3.9 μL/minute and 4.1 μL/minute, between 3.9 μL/minute and 4.2 μL/minute, between 4.0 μL/minute and 4.1 μL/minute, between 4.0 μL/minute and 4.2 μL/minute, between 4.0 μL/minute and 4.3 μL/minute, between 4.1 μL/minute and 4.2 μL/minute, between 4.1 μL/minute and 4.3 μL/minute, between 4.1 μL/minute and 4.4 μL/minute, between 4.2 μL/minute and 4.3 μL/minute, between 4.2 μL/minute and 4.4 μL/minute, between 4.2 μL/minute and 4.5 μL/minute, between 4.3 μL/minute and 4.4 μL/minute, between 4.3 μL/minute and 4.5 μL/minute, between 4.3 μL/minute and 4.6 μL/minute, between 4.4 μL/minute and 4.5 μL/minute, between 4.4 μL/minute and 4.6 μL/minute, between 4.4 μL/minute and 4.7 μL/minute, between 4.5 μL/minute and 4.6 μL/minute, between 4.5 μL/minute and 4.7 μL/minute, between 4.5 μL/minute and 4.8 μL/minute, between 4.6 μL/minute and 4.7 μL/minute, between 4.6 μL/minute and 4.8 μL/minute, between 4.6 μL/minute and 4.9 μL/minute, between 4.7 μL/minute and 4.8 μL/minute, between 4.7 μL/minute and 4.9 μL/minute, between 4.7 μL/minute and 5.0 μL/minute, 4.8 μL/minute and 4.9 μL/minute, between 4.8 μL/minute and 5.0 μL/minute, or between 4.9 μL/minute and 5.0 μL/minute.

The viral vector {e.g., an AAV including (i) exogenous nucleic acid encoding any of the polypeptides, or any combination of polypeptides, described herein (e.g., NeuroD1, Isl1, Lhx3, Dlx2, and Ngn2) and/or (ii) exogenous nucleic acid encoding mir124 and mir218 can be administered in an amount corresponding to a dose of virus in the range of about 1.0×10¹⁰ vg/kg to about 1.0×10¹⁴ vg/kg (virus genomes per kg body weight). In some cases, the viral vector (e.g., an AAV including (i) exogenous nucleic acid encoding any of the polypeptides, or any combination of polypeptides, described herein (e.g., NeuroD1, Isl1, Lhx3, Dlx2, and Ngn2) and/or (ii) exogenous nucleic acid encoding mir124 and mir218} can be administered in amount corresponding to a dose of virus in the range of about 1.0×10¹¹ to about 1.0×10¹² vg/kg, a range of about 5.0×10¹¹ to about 5.0×10¹² vg/kg, or a range of about 1.0×10¹² to about 5.0×10¹¹ is still more preferred. In some cases, the viral vector is administered in an amount corresponding to a dose of about 2.5×10¹² vg/kg. In some cases, the effective amount of the viral vector {e.g., an AAV including (i) exogenous nucleic acid encoding any of the polypeptides, or any combination of polypeptides, described herein (e.g., NeuroD1, Isl1, Lhx3, Dlx2, and Ngn2) and/or (ii) exogenous nucleic acid encoding mir124 and mir218} can be a volume of about 1 μL to about 500 μL, corresponding to the volume for the vg/kg (virus genomes per kg body weight) doses described herein. In some cases, the amount of the viral vector to be administered {e.g., an AAV including (i) exogenous nucleic acid encoding any of the polypeptides, or any combination of polypeptides, described herein (e.g., NeuroD1, Isl1, Lhx3, Dlx2, and Ngn2) and/or (ii) exogenous nucleic acid encoding mir124 and mir218} is adjusted according to the strength of the expression of one or more transgenes (e.g., NeuroD1).

In some cases, the effective volume administered of the viral vector is between 1 μL and 25 μL, between 1 μL and 50 μL, between 1 μL and 75 μL, between 25 μL and 50 μL, between 25 L and 75 μL, between 25 μL and 100 μL, between 50 μL and 75 μL, between 50 μL and 100 μL, between 50 μL and 125 μL, between 75 μL and 100 μL, between 75 μL and 125 μL, between 75 μL and 150 μL, between 100 μL and 125 μL, between 100 μL and 150 μL, between 100 μL and 175 μL, between 125 μL and 150 μL, between 125 μL and 175 μL, between 125 μL and 200 μL, between 150 μL and 175 μL, between 150 μL and 200 μL, between 150 μL and 225 μL, between 175 μL and 200 μL, between 175 μL and 225 μL, between 175 μL and 250 μL, between 200 μL and 225 μL, between 200 μL and 250 μL, between 200 μL and 275 μL, between 225 μL and 250 μL, between 225 μL and 275 μL, between 225 μL and 300 μL, between 250 μL and 275 μL, between 250 μL and 300 μL, between 250 μL and 325 μL, between 275 μL and 300 μL, between 275 μL and 325 μL, between 275 μL and 350 μL, between 300 μL and 325 μL, between 300 μL and 350 μL, between 300 μL and 375 μL, between 325 μL and 350 μL, between 325 μL and 375 μL, between 325 μL and 400 μL, between 350 μL and 375 μL, between 350 μL and 400 μL, between 350 μL and 425 μL, between 375 μL and 400 μL, between 375 μL and 425 μL, between 375 μL and 450 μL, between 400 μL and 425 μL, between 400 μL and 450 μL, between 400 μL and 475 μL, between 425 μL and 450 μL, between 425 μL and 475 μL, between 425 μL and 500 μL, between 450 μL and 475 μL, between 450 μL and 500 μL, or between 475 μL and 500 μL.

In some cases, an adeno-associated virus vector including (i) exogenous nucleic acid encoding any of the polypeptides, or any combination of polypeptides, described herein {e.g., NeuroD1, Isl1, Lhx3, Dlx2, and Ngn2) and/or (ii) exogenous nucleic acid encoding mir124 and mir218 under transcriptional control of a ubiquitous (constitutive) promoter or a neuron-specific promoter wherein the nucleic acid sequence encoding the polypeptides and miRNA (e.g., NeuroD1, Isl1, Lhx3, Dlx2, mir124, Ngn2, and mir218} or any combination thereof is inverted and in the wrong orientation for expression of the transgene and further includes sites for recombinase activity by a site specific recombinase, until the site-specific recombinase inverts the inverted nucleic acid sequence encoding the transgene, thereby allowing expression of the transgene, is delivered by stereotactic injection into the brain or through local delivery to a spinal cord injury of a subject along with an adeno-associated virus encoding a site specific recombinase.

In some cases, an adeno-associated virus vector including (i) exogenous nucleic acid encoding any of the polypeptides, or any combination of polypeptides, described herein (e.g., NeuroD1, Isl1, Lhx3, Dlx2, and Ngn2) and/or (ii) exogenous nucleic acid encoding mir124 and mir218 under transcriptional control of a ubiquitous (constitutive) promoter or a neuron-specific promoter wherein the nucleic acid sequence encoding the transgene is inverted and in the wrong orientation for expression of the transgene and further includes sites for recombinase activity by a site specific recombinase, until the site-specific recombinase inverts the inverted nucleic acid sequence encoding the transgene, thereby allowing expression of the transgene, is delivered by stereotactic injection into the brain or by local delivery to the spinal cord of a subject along with an adeno-associated virus encoding a site specific recombinase according to the methods described herein.

In some cases, the site-specific recombinase is Cre recombinase and the sites for recombinase activity are recognition sites loxP and lox2272 sites.

In some cases, treatment of a subject with (i) exogenous nucleic acid encoding any of the polypeptides, or any combination of polypeptides, described herein (e.g., NeuroD1, Isl1, Lhx3, Dlx2, and Ngn2) and/or (ii) exogenous nucleic acid encoding mir124 and mir218 is monitored during or after treatment to monitor progress and/or final outcome of the treatment. Post-Treatment Assay for successful neuronal cell integration and restoration of tissue microenvironment is diagnosed by restoration or near-restoration of normal electrophysiology, blood flow, tissue structure, and function. Non-invasive methods to assay neural function include EEG. Blood flow may be non-invasively assayed via Near Infrared Spectroscopy and fMRI. Non-invasive methods to assay tissue structure include MRI, CAT scan, PET scan, or ultrasound. Behavioral assays may be used to non-invasively assay for restoration of brain function. The behavioral assay should be matched to the loss of function caused by original brain injury. For example, if injury caused paralysis, the patient's mobility and limb dexterity should be tested. If injury caused loss or slowing of speech, patient's ability to communicate via spoken word should be assayed. Restoration of normal behavior post NeuroD1 treatment alone or in combination with Dlx2 indicates successful creation and integration of effective neuronal circuits. These methods may be used singularly or in any combination to assay for neural function and tissue health. Assays to evaluate treatment may be performed at any point, such as 1 day, 2 days, 3 days, one week, 2 weeks, 3 weeks, one month, two months, three months, six months, one year, or later, after NeuroD1 treatment alone or in combination with Dlx2. Such assays may be performed prior to NeuroD1 treatment alone or in combination with Dlx2 in order to establish a baseline comparison if desired.

Scientific and technical terms used herein are intended to have the meanings commonly understood by those of ordinary skill in the art. Such terms are found defined and used in context in various standard references illustratively including J. Sambrook and D. W. Russell, Molecular Cloning: A Laboratory Manual, Cold Spring Harbor Laboratory Press; 3rd Ed., 2001; F. M. Asubel, Ed., Short Protocols in Molecular Biology, Current Protocols; 5th Ed., 2002; B. Alberts et al., Molecular Biology of the Cell, 4th Ed., Garland, 2002; D. L. Nelson and M. M. Cox, Lehninger Principles of Biochemistry, 4th Ed., W.H. Freeman & Company, 2004; Engelke, D. R., RNA Interference (RNAi): Nuts and Bolts of RNAi Technology, DNA Press LLC, Eagleville, PA, 2003; Herdewijn, p. (Ed.), Oligonucleotide Synthesis: Methods and Applications, Methods in Molecular Biology, Humana Press, 2004; A. Nagy, M. Gertsenstein, K. Vintersten, R. Behringer, Manipulating the Mouse Embryo: A Laboratory Manual, Cold Spring Harbor Laboratory Press, 3rd Ed.; Dec. 15, 2002, ISBN-10:0879695919; Kursad Turksen (Ed.), Embryonic Stem Cells: Methods and Protocols in Methods in Molecular Biology, 2002; 185, Human Press: Current Protocols in Stem Cell Biology, ISBN:9780470151808.

As used herein, the singular terms “a,” “an,” and “the” are not intended to be limiting and include plural referents unless explicitly stated otherwise or the context clearly indicates otherwise.

As used herein, the term “NeuroD1 protein” refers to a bHLH proneural transcription factor involved in embryonic brain development and in adult neurogenesis, see Cho et al., Mol. Neurobiol., 30:35-47 (2004); Kuwabara et al., Nature Neurosci., 12:1097-1105 (2009); and Gao et al., Nature Neurosci., 12:1090-1092 (2009). NeuroD1 is expressed late in development, mainly in the nervous system and is involved in neuronal differentiation, maturation and survival.

As used herein, the term “Dlx2 protein” refers to a distal-less Homeobox 2 transcription factor that has a role in forebrain and craniofacial development. An example of a human Dlx2 polypeptide includes, without limitation, NCBI reference sequence: NP_004396.1 or a biologically active fragment thereof.

In some embodiments, the Dlx2 polypeptide includes an amino acid substitution, insertion, or deletion that results in increased activity of the mutated Dlx2 as compared to the wildtype Dlx2 polypeptide (e.g., NCBI reference sequence: NP_004396.1).

As used herein, the term “Isl1” refers to ISL LIM Homeobox 1 transcription factor involved in regulating insulin gene expression and required for motor neuron generation. An example of a human Isl1 polypeptide includes, without limitation, NCBI reference sequence: NP_002193.2 or a biologically active fragment thereof. In some embodiments, the Isl1 polypeptide includes an amino acid substitution, insertion, or deletion that results in increased activity of the mutated Isl1 as compared to the wildtype Isl1 polypeptide (e.g., NCBI reference sequence: NP_002193.2).

The term “NeuroD1 protein” or “exogenous NeuroD1” encompasses human NeuroD1 protein, identified here as SEQ ID NO: 2 and mouse NeuroD1 protein, identified here as SEQ ID NO: 4. In addition to the NeuroD1 protein of SEQ ID NO: 2 and SEQ ID NO: 4, the term “NeuroD1 protein” encompasses variants of NeuroD1 protein, such as variants of SEQ ID NO: 2 and SEQ ID NO: 4, which may be included in a method described herein.

The term “Dlx2 protein” or “exogenous Dlx2” encompasses human Dlx2 protein, identified here as SEQ ID NO: 11 and mouse Dlx2 protein, identified here as SEQ ID NO: 13. In addition to the Dlx2 protein of SEQ ID NO: 11 and SEQ ID NO: 13, the term “Dlx2 protein” encompasses variants of Dlx2 protein, such as variants of SEQ ID NO: 11 and SEQ ID NO: 13, which may be included in a method described herein.

The term “Isl1 protein” or “exogenous Isl1” encompasses human Isl1 protein, identified here as SEQ ID NO: 15 and mouse Isl1 protein, identified here as SEQ ID NO: 17. In addition to the Isl1 protein of SEQ ID NO: 15 and SEQ ID NO: 17, the term “Isl1 protein” encompasses variants of Isl1 protein, such as variants of SEQ ID NO: 15 and SEQ ID NO: 17, which may be included in a method described herein. In some cases, an Isl1 protein can include the sequence set forth in GenBank Accession No. EAW54861, NP_002193.2, or AAH31213.1.

As used herein, the term “variant” refers to naturally occurring genetic variations and recombinantly prepared variations, each of which contain one or more changes in its amino acid sequence compared to a reference NeuroD1 protein, such as SEQ ID NO: 2 or SEQ ID NO: 4. In some cases, the term “variant” refers to naturally occurring genetic variations and recombinantly prepared variations, each of which contain one or more changes in its amino acid sequence compared to a reference Dlx2 protein, such as SEQ ID NO: 11 or SEQ ID NO: 13. In some cases, the term “variant” refers to naturally occurring genetic variations and recombinantly prepared variations, each of which contain one or more changes in its amino acid sequence compared to a reference Isl1 protein, such as SEQ ID NO: 15 or SEQ ID NO: 17. Such changes include those in which one or more amino acid residues have been modified by amino acid substitution, addition or deletion. The term “variant” encompasses orthologs of human NeuroD1, including for example mammalian and bird NeuroD1, such as, but not limited to NeuroD1 orthologs from a non-human primate, cat, dog, sheep, goat, horse, cow, pig, bird, poultry animal and rodent such as but not limited to mouse and rat. In a non-limiting example, mouse NeuroD1, exemplified herein as amino acid sequence SEQ ID NO: 4 is an ortholog of human NeuroD1. In some cases, preferred variants have at least 75%, 80%, 85%, 90%, 91%, 92%, 93%, 94%, 95%, 96%, 97%, 98%, or 99% identity to SEQ ID NO: 2 or SEQ ID NO: 4.

In some cases, the term “variant” encompasses orthologs of human Dlx2, including for example mammalian and bird Dlx2, such as, but not limited to Dlx2 orthologs from a non-human primate, cat, dog, sheep, goat, horse, cow, pig, bird, poultry animal and rodent such as but not limited to mouse and rat. In a non-limiting example, mouse Dlx2, exemplified herein as amino acid sequence SEQ ID NO: 13 is an ortholog of human Dlx2. In some cases, preferred variants have at least 75%, 80%, 85%, 90%, 91%, 92%, 93%, 94%, 95%, 96%, 97%, 98%, or 99% identity to SEQ ID NO: 11 or SEQ ID NO: 13.

Mutations can be introduced using standard molecular biology techniques, such as site-directed mutagenesis and PCR-mediated mutagenesis. One of skill in the art will recognize that one or more amino acid mutations can be introduced without altering the functional properties of the NeuroD1 protein, Dlx2 protein, or Isl1 protein. For example, one or more amino acid substitutions, additions, or deletions can be made without altering the functional properties of the NeuroD1 protein of SEQ ID NO: 2 or 4. In some cases, one or more amino acid substitutions, additions, or deletions can be made without altering the functional properties of the Dlx2 protein of SEQ ID NO: 11 or 13. In some cases, one or more amino acid substitutions, additions, or deletions can be made without altering the functional properties of the Isl1 protein of SEQ ID NO: 15 or 17.

Conservative amino acid substitutions can be made in a NeuroD1 protein to produce a NeuroD1 protein variant. In some cases, conservative amino acid substitutions can be made in a Dlx2 protein to produce a Dlx2 protein variant. In some cases, conservative amino acid substitutions can be made in a Isl1 protein to produce a Isl1 protein variant. Conservative amino acid substitutions are art recognized substitutions of one amino acid for another amino acid having similar characteristics. For example, each amino acid may be described as having one or more of the following characteristics: electropositive, electronegative, aliphatic, aromatic, polar, hydrophobic and hydrophilic. A conservative substitution is a substitution of one amino acid having a specified structural or functional characteristic for another amino acid having the same characteristic. Acidic amino acids include aspartate, glutamate; basic amino acids include histidine, lysine, arginine; aliphatic amino acids include isoleucine, leucine and valine; aromatic amino acids include phenylalanine, glycine, tyrosine and tryptophan; polar amino acids include aspartate, glutamate, histidine, lysine, asparagine, glutamine, arginine, serine, threonine and tyrosine; and hydrophobic amino acids include alanine, cysteine, phenylalanine, glycine, isoleucine, leucine, methionine, proline, valine and tryptophan; and conservative substitutions include substitution among amino acids within each group. Amino acids may also be described in terms of relative size, alanine, cysteine, aspartate, glycine, asparagine, proline, threonine, serine, valine, all typically considered to be small.

NeuroD1 variants can include synthetic amino acid analogs, amino acid derivatives and/or non-standard amino acids, illustratively including, without limitation, alpha-aminobutyric acid, citrulline, canavanine, cyanoalanine, diaminobutyric acid, diaminopimelic acid, dihydroxy-phenylalanine, djenkolic acid, homoarginine, hydroxyproline, norleucine, norvaline, 3-phosphoserine, homoserine, 5-hydroxytryptophan, 1-methylhistidine, 3-methylhistidine, and ornithine. In some cases, Dlx2 variants can include synthetic amino acid analogs, amino acid derivatives and/or non-standard amino acids, illustratively including, without limitation, alpha-aminobutyric acid, citrulline, canavanine, cyanoalanine, diaminobutyric acid, diaminopimelic acid, dihydroxy-phenylalanine, djenkolic acid, homoarginine, hydroxyproline, norleucine, norvaline, 3-phosphoserine, homoserine, 5-hydroxytryptophan, 1-methylhistidine, 3-methylhistidine, and ornithine. In some cases, Isl1 variants can include synthetic amino acid analogs, amino acid derivatives and/or non-standard amino acids, illustratively including, without limitation, alpha-aminobutyric acid, citrulline, canavanine, cyanoalanine, diaminobutyric acid, diaminopimelic acid, dihydroxy-phenylalanine, djenkolic acid, homoarginine, hydroxyproline, norleucine, norvaline, 3-phosphoserine, homoserine, 5-hydroxytryptophan, 1-methylhistidine, 3-methylhistidine, and ornithine.

To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions×100%). In one embodiment, the two sequences are the same length.

The determination of percent identity between two sequences can also be accomplished using a mathematical algorithm. A preferred, non-limiting example of a mathematical algorithm utilized for the comparison of two sequences is the algorithm of Karlin and Altschul, PNAS, 87:2264-2268 (1990), modified as in Karlin and Altschul, PNAS, 90:5873-5877 (1993). Such an algorithm is incorporated into the NBLAST and XBLAST programs of Altschul et al., J. Mol. Biol., 215:403 (1990). BLAST nucleotide searches are performed with the NBLAST nucleotide program parameters set, e.g., for score=100, wordlength=12 to obtain nucleotide sequences homologous to a nucleic acid molecule described herein.

BLAST protein searches are performed with the XBLAST program parameters set, e.g., to score 50, wordlength=3 to obtain amino acid sequences homologous to a protein molecule of the present invention. To obtain gapped alignments for comparison purposes, Gapped BLAST are utilized as described in Altschul et al. (Nucleic Acids Res., 25:3389-3402 (1997)). Alternatively, PSI BLAST is used to perform an iterated search which detects distant relationships between molecules. When utilizing BLAST, Gapped BLAST, and PSI Blast programs, the default parameters of the respective programs (e.g., of XBLAST and NBLAST) are used (see, e.g., the NCBI website).

Another preferred, non-limiting example of a mathematical algorithm utilized for the comparison of sequences is the algorithm of Myers and Miller (CABIOS, 4:11-17 (1988)). Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 is used.

The percent identity between two sequences is determined using techniques similar to those described above, with or without allowing gaps. In calculating percent identity, typically only exact matches are counted.

The term “NeuroD1 protein” encompasses fragments of the NeuroD1 protein, such as fragments of SEQ ID NOs. 2 and 4 and variants thereof, operable in a method or composition described herein.

The term “Dlx2 protein” encompasses fragments of the Dlx2 protein, such as fragments of SEQ ID NOs: 11 and 13 and variants thereof, operable in a method or composition described herein.

The term “Isl1 protein” encompasses fragments of the Isl1 protein, such as fragments of SEQ ID NOs: 15 and 17 and variants thereof, operable in a method or composition described herein.

NeuroD1 proteins and nucleic acids may be isolated from natural sources, such as the brain of an organism or cells of a cell line which expresses NeuroD1. Alternatively, NeuroD1 protein or nucleic acid may be generated recombinantly, such as by expression using an expression construct, in vitro or in vivo. NeuroD1 proteins and nucleic acids may also be synthesized by well-known methods. In some cases, Dlx2 proteins and nucleic acids may be isolated from natural sources, such as the brain of an organism or cells of a cell line which expresses Dlx2. Alternatively, Dlx2 protein or nucleic acid may be generated recombinantly, such as by expression using an expression construct, in vitro or in vivo. Dlx2 proteins and nucleic acids may also be synthesized by well-known methods. In some cases, Isl1 proteins and nucleic acids may be isolated from natural sources, such as the brain of an organism or cells of a cell line which expresses Isl1. Alternatively, Isl1 protein or nucleic acid may be generated recombinantly, such as by expression using an expression construct, in vitro or in vivo. Isl1 proteins and nucleic acids may also be synthesized by well-known methods.

NeuroD1 included in a method or composition described herein can be produced using recombinant nucleic acid technology. Recombinant NeuroD1 production includes introducing a recombinant expression vector encompassing a DNA sequence encoding NeuroD1 into a host cell. In some cases, Dlx2 included in a method or composition described herein can be produced using recombinant nucleic acid technology. Recombinant Dlx2 production includes introducing a recombinant expression vector encompassing a DNA sequence encoding Dlx2 into a host cell. In some cases, Isl1 included in a method or composition described herein can be produced using recombinant nucleic acid technology. Recombinant Isl1 production includes introducing a recombinant expression vector encompassing a DNA sequence encoding Isl1 into a host cell.

In some cases, a nucleic acid sequence encoding NeuroD1 introduced into a host cell to produce NeuroD1 can encode SEQ ID NO: 2, SEQ ID NO: 4, or a variant thereof.

In some cases, a nucleic acid sequence encoding Dlx2 introduced into a host cell to produce Dlx2 can encode SEQ ID NO: 11, SEQ ID NO: 13, or a variant thereof.

In some cases, a nucleic acid sequence encoding Isl1 introduced into a host cell to produce Isl1 can encode SEQ ID NO: 15, SEQ ID NO: 17, or a variant thereof.

In some cases, the nucleic acid sequence identified herein as SEQ ID NO: 1 encodes SEQ ID NO: 2 and is included in an expression vector and expressed to produce NeuroD1. In some cases, the nucleic acid sequence identified herein as SEQ ID NO: 10 encodes SEQ ID NO: 11 and is included in an expression vector and expressed to produce Dlx2. In some cases, the nucleic acid sequence identified herein as SEQ ID NO: 14 encodes SEQ ID NO: 15 and is included in an expression vector and expressed to produce Isl1. In some cases, the nucleic acid sequence identified herein as SEQ ID NO: 3 encodes SEQ ID NO: 4 and is included in an expression vector and expressed to produce NeuroD1. In some cases, the nucleic acid sequence identified herein as SEQ ID NO: 12 encodes SEQ ID NO: 13 and is included in an expression vector and expressed to produce Dlx2. In some cases, the nucleic acid sequence identified herein as SEQ ID NO: 16 encodes SEQ ID NO: 17 and is included in an expression vector and expressed to produce Isl1.

It is appreciated that due to the degenerate nature of the genetic code, nucleic acid sequences substantially identical to SEQ ID NOs. 1 and 3 encode NeuroD1 and variants of NeuroD1, and that such alternate nucleic acids may be included in an expression vector and expressed to produce NeuroD1 and variants of NeuroD1. In some cases, nucleic acid sequences substantially identical to SEQ ID NOs: 10 and 12 encode Dlx2 and variants of Dlx2, and that such alternate nucleic acids may be included in an expression vector and expressed to produce Dlx2 and variants of Dlx2. In some cases, nucleic acid sequences substantially identical to SEQ ID NOs: 14 and 16 encode Isl1 and variants of Isl1, and that such alternate nucleic acids may be included in an expression vector and expressed to produce Isl1 and variants of Isl1. One of skill in the art will appreciate that a fragment of a nucleic acid encoding NeuroD1 protein can be used to produce a fragment of a NeuroD1 protein. In some cases, one of skill in the art will appreciate that a fragment of a nucleic acid encoding Dlx2 protein can be used to produce a fragment of a Dlx2 protein. In some cases, one of skill in the art will appreciate that a fragment of a nucleic acid encoding Isl1 protein can be used to produce a fragment of a Isl1 protein.

As used herein, the term “Lhx3” refers to LIM Homeobox 3 transcription factor involved in pituitary development and motor neuron specification. An example of a human Lhx3 polypeptide includes, without limitation, NCBI reference sequence: NP_001350675.1 or a biologically active fragment thereof. In some embodiments, the Lhx3 polypeptide includes an amino acid substitution, insertion, or deletion that results in increased activity of the mutated Lhx3 as compared to the wildtype Lhx3 polypeptide (e.g., NCBI reference sequence: NP_001350675.1).

As used herein, the term “mir124” refers to microRNA 124. MicroRNAs (miRNAs) are short (20-24 nt) non-coding RNAs that are involved in post-transcriptional regulation of gene expression in multicellular organisms by affecting both the stability and translation of mRNAs.

As used herein, the term “mir218” refers to microRNA 218. MicroRNAs (miRNAs) are short (20-24 nt) non-coding RNAs that are involved in post-transcriptional regulation of gene expression in multicellular organisms by affecting both the stability and translation of mRNAs.

As used herein, the term “Ngn2” refers to neural-specific basic helix-loop-helix (bHLH) transcription factor that can specify a neuronal fate on ectodermal cells and is expressed in neural progenitor cells within the developing central and peripheral nervous systems. An example of a human Ngn2 polypeptide includes, without limitation, NCBI reference sequence: NP_076924.1 or a biologically active fragment thereof. In some embodiments, the Ngn2 polypeptide includes an amino acid substitution, insertion, or deletion that results in increased activity of the mutated Ngn2 as compared to the wildtype Ngn2 polypeptide (e.g., NCBI reference sequence: NP_076924.1).

An expression vector contains a nucleic acid that includes segment encoding a polypeptide of interest operably linked to one or more regulatory elements that provide for transcription of the segment encoding the polypeptide of interest. The term “operably linked” as used herein refers to a nucleic acid in functional relationship with a second nucleic acid. The term “operably linked” encompasses functional connection of two or more nucleic acid molecules, such as a nucleic acid to be transcribed and a regulatory element. The term “regulatory element” as used herein refers to a nucleotide sequence which controls some aspect of the expression of an operably linked nucleic acid. Exemplary regulatory elements include an enhancer, such as, but not limited to: woodchuck hepatitis virus posttranscriptional regulatory element (WPRE); an internal ribosome entry site (IRES) or a 2A domain; an intron; an origin of replication; a polyadenylation signal (pA); a promoter; a transcription termination sequence; and an upstream regulatory domain, which contribute to the replication, transcription, post-transcriptional processing of an operably linked nucleic acid sequence. Those of ordinary skill in the art are capable of selecting and using these and other regulatory elements in an expression vector with no more than routine experimentation.

The term “promoter” as used herein refers to a DNA sequence operably linked to a nucleic acid sequence to be transcribed such as a nucleic acid sequence encoding NeuroD1. A promoter is generally positioned upstream of a nucleic acid sequence to be transcribed and provides a site for specific binding by RNA polymerase and other transcription factors. In specific embodiments, a promoter is generally positioned upstream of the nucleic acid sequence transcribed to produce the desired molecule, and provides a site for specific binding by RNA polymerase and other transcription factors.

As will be recognized by the skilled artisan, the 5′ non-coding region of a gene can be isolated and used in its entirety as a promoter to drive expression of an operably linked nucleic acid. Alternatively, a portion of the 5′ non-coding region can be isolated and used to drive expression of an operably linked nucleic acid. In general, about 500-6000 bp of the 5′ non-coding region of a gene is used to drive expression of the operably linked nucleic acid. Optionally, a portion of the 5′ non-coding region of a gene containing a minimal amount of the 5′ non-coding region needed to drive expression of the operably linked nucleic acid is used. Assays to determine the ability of a designated portion of the 5′ non-coding region of a gene to drive expression of the operably linked nucleic acid are well-known in the art.

Particular promoters used to drive expression (i) exogenous nucleic acid encoding any of the polypeptides, or any combination of polypeptides, described herein (e.g., NeuroD1, Isl1, Lhx3, Dlx2, and Ngn2) and/or (ii) exogenous nucleic acid encoding mir124 and mir218 according to methods described herein are “ubiquitous” or “constitutive” promoters, that drive expression in many, most, or all cell types of an organism into which the expression vector is transferred. Non-limiting examples of ubiquitous promoters that can be used in expression of (i) exogenous nucleic acid encoding any of the polypeptides, or any combination of polypeptides, described herein (e.g., NeuroD1, Isl1, Lhx3, Dlx2, and Ngn2) and/or (ii) exogenous nucleic acid encoding mir124 and mir218 are cytomegalovirus promoter; simian virus 40 (SV40) early promoter; rous sarcoma virus promoter; adenovirus major late promoter; beta actin promoter; glyceraldehyde 3-phosphate dehydrogenase; glucose-regulated protein 78 promoter; glucose-regulated protein 94 promoter; heat shock protein 70 promoter; beta-kinesin promoter; ROSA promoter; ubiquitin B promoter; eukaryotic initiation factor 4A1 promoter and elongation Factor I promoter; all of which are well-known in the art and which can be isolated from primary sources using routine methodology or obtained from commercial sources. Promoters can be derived entirely from a single gene or can be chimeric, having portions derived from more than one gene.

Combinations of regulatory sequences may be included in an expression vector and used to drive expression of NeuroD1. A non-limiting example included in an expression vector to drive expression of NeuroD1 is the CAG promoter which combines the cytomegalovirus CMV early enhancer element and chicken beta-actin promoter.

Particular promoters used to drive expression of NeuroD1 (or any other (i) exogenous nucleic acid encoding any of the polypeptides, or any combination of polypeptides, described herein (e.g., NeuroD1, Isl1, Lhx3, Dlx2, and Ngn2) and/or (ii) exogenous nucleic acid encoding mir124 and mir218 described herein according to methods described herein are those that drive expression preferentially in glial cells, particularly astrocytes and/or NG2 cells. Such promoters are termed “astrocyte-specific” and/or “NG2 cell-specific” promoters.

Non-limiting examples of astrocyte-specific promoters are glial fibrillary acidic protein (GFAP) promoter and aldehyde dehydrogenase 1 family, member L1 (AldhlL1) promoter. Human GFAP promoter is shown herein as SEQ ID NO:6. Mouse AldhlL1 promoter is shown herein as SEQ ID NO:7.

A non-limiting example of an NG2 cell-specific promoter is the promoter of the chondroitin sulfate proteoglycan 4 gene, also known as neuron-glial antigen 2 (NG2). Human NG2 promoter is shown herein as SEQ ID NO:8.

Particular promoters used to drive expression of NeuroD1 according to methods described herein are those that drive expression preferentially in reactive glial cells, particularly reactive astrocytes and/or reactive NG2 cells. Such promoters are termed “reactive astrocyte-specific” and/or “reactive NG2 cell-specific” promoters.

Particular promoters used to drive expression of Dlx2 according to methods described herein are those that drive expression preferentially in reactive glial cells, particularly reactive astrocytes and/or reactive NG2 cells. Such promoters are termed “reactive astrocyte-specific” and/or “reactive NG2 cell-specific” promoters.

A non-limiting example of a “reactive astrocyte-specific” promoter is the promoter of the lipocalin 2 (lcn2) gene. Mouse lcn2 promoter is shown herein as SEQ ID NO:5.

Homologues and variants of ubiquitous and cell type-specific promoters may be used in expressing NeuroD1.

In some cases, promoter homologues and promoter variants can be included in an expression vector for expressing NeuroD1. In some cases, promoter homologues and promoter variants can be included in an expression vector for expressing Dlx2. In some cases, promoter homologues and promoter variants can be included in an expression vector for expressing Isl1. The terms “promoter homologue” and “promoter variant” refer to a promoter which has substantially similar functional properties to confer the desired type of expression, such as cell type-specific expression of NeuroD1 or ubiquitous expression of NeuroD1, on an operably linked nucleic acid encoding NeuroD1 compared to those disclosed herein. For example, a promoter homologue or variant has substantially similar functional properties to confer cell type-specific expression on an operably linked nucleic acid encoding NeuroD1 compared to GFAP, S100b, AldhlL1, NG2, lcn2 and CAG promoters.

One of skill in the art will recognize that one or more nucleic acid mutations can be introduced without altering the functional properties of a given promoter. Mutations can be introduced using standard molecular biology techniques, such as site-directed mutagenesis and PCR-mediated mutagenesis, to produce promoter variants. As used herein, the term “promoter variant” refers to either an isolated naturally occurring or a recombinantly prepared variation of a reference promoter, such as, but not limited to, GFAP, S100b, AldhlL1, NG2, lcn2 and pCAG promoters.

It is known in the art that promoters from other species are functional, e.g. the mouse AldhlL1promoter is functional in human cells. Homologues and homologous promoters from other species can be identified using bioinformatics tools known in the art, see for example, Xuan et al., Genome Biol., 6:R72 (2005); Zhao et al., Nucl. Acid Res., 33:D103-107 (2005); and Halees et al., Nucl. Acids. Res., 31:3554-3559 (2003).

Structurally, homologues and variants of cell type-specific promoters of NeuroD1 or and/or ubiquitous promoters have at least 80%, 81%, 82%, 83%, 84%, 85%, 86%, 87%, 88%, 89, 90%, 91%, 92%, 93%, 94%, 95%, 96%, 97%, 98%, 99%, or greater, nucleic acid sequence identity to the reference developmentally regulated and/or ubiquitous promoter and include a site for binding of RNA polymerase and, optionally, one or more binding sites for transcription factors.

A nucleic acid sequence which is substantially identical to SEQ ID NO:1 or SEQ ID NO:3 is characterized as having a complementary nucleic acid sequence capable of hybridizing to SEQ ID NO:1 or SEQ ID NO:3 under high stringency hybridization conditions.

In addition to one or more nucleic acids encoding NeuroD1, one or more nucleic acid sequences encoding additional proteins can be included in an expression vector. For example, such additional proteins include non-NeuroD1 proteins such as reporters, including, but not limited to, beta-galactosidase, green fluorescent protein and antibiotic resistance reporters.

In particular embodiments, the recombinant expression vector encodes at least NeuroD1 of SEQ ID NO:2, a protein having at least 95% identity to SEQ ID NO:2, or a protein encoded by a nucleic acid sequence substantially identical to SEQ ID NO:1.

In particular embodiments, the recombinant expression vector encodes at least NeuroD1 of SEQ ID NO:4, a protein having at least 95% identity to SEQ ID NO:4, or a protein encoded by a nucleic acid sequence substantially identical to SEQ ID NO:2.

SEQ ID NO:9 is an example of a nucleic acid including CAG promoter operably linked to a nucleic acid encoding NeuroD1, and further including a nucleic acid sequence encoding EGFP and an enhancer, WPRE. An IRES separates the nucleic acid encoding NeuroD1 and the nucleic acid encoding EGFP. SEQ ID NO:9 is inserted into an expression vector for expression of NeuroD1 and the reporter gene EGFP. Optionally, the IRES and nucleic acid encoding EGFP are removed and the remaining CAG promoter and operably linked nucleic acid encoding NeuroD1 is inserted into an expression vector for expression of NeuroD1. The WPRE or another enhancer is optionally included.

Optionally, a reporter gene is included in a recombinant expression vector encoding NeuroD1. A reporter gene may be included to produce a peptide or protein that serves as a surrogate marker for expression of NeuroD1 from the recombinant expression vector. In some cases, a reporter gene is included in a recombinant expression vector encoding Dlx2. In some cases, a reporter gene is included in a recombinant expression vector encoding Isl1. A reporter gene may be included to produce a peptide or protein that serves as a surrogate marker for expression of Dlx2 from the recombinant expression vector. The term “reporter gene” as used herein refers to gene that is easily detectable when expressed, for example by chemiluminescence, fluorescence, colorimetric reactions, antibody binding, inducible markers and/or ligand binding assays. Exemplary reporter genes include, but are not limited to, green fluorescent protein (GFP), enhanced green fluorescent protein (eGFP), yellow fluorescent protein (YFP), enhanced yellow fluorescent protein (eYFP), cyan fluorescent protein (CFP), enhanced cyan fluorescent protein (eCFP), blue fluorescent protein (BFP), enhanced blue fluorescent protein (eBFP), MmGFP (Zernicka-Goetz et al., Development, 124:1133-1137 (1997)), dsRed, luciferase and beta-galactosidase (lacZ).

The process of introducing genetic material into a recipient host cell, such as for transient or stable expression of a desired protein encoded by the genetic material in the host cell is referred to as “transfection.” Transfection techniques are well-known in the art and include, but are not limited to, electroporation, particle accelerated transformation also known as “gene gun” technology, liposome-mediated transfection, calcium phosphate or calcium chloride co-precipitation-mediated transfection, DEAE-dextran-mediated transfection, microinjection, polyethylene glycol mediated transfection, heat shock mediated transfection and virus-mediated transfection. As noted herein, virus-mediated transfection may be accomplished using a viral vector such as those derived from adenovirus, adeno-associated virus and lentivirus.

Optionally, a host cell is transfected ex-vivo and then re-introduced into a host organism. For example, cells or tissues may be removed from a subject, transfected with an expression vector encoding NeuroD1 and then returned to the subject. In some cases, cells or tissues may be removed from a subject, transfected with an expression vector encoding NeuroD1 and Dlx2 and then returned to the subject. In some cases, cells or tissues may be removed from a subject, transfected with an expression vector encoding NeuroD1 and an expression vector encoding Dlx2 and then returned to the subject. In some cases, cells or tissues may be removed from a subject, transfected with an expression vector encoding NeuroD1 and Isl1 and then returned to the subject. In some cases, cells or tissues may be removed from a subject, transfected with an expression vector encoding NeuroD1 and an expression vector encoding Isl1 and then returned to the subject.

Introduction of a recombinant expression vector including a nucleic acid encoding NeuroD1, or a functional fragment thereof, into a host glial cell in vitro or in vivo for expression of exogenous NeuroD1 in the host glial cell to convert the glial cell to a neuron is accomplished by any of various transfection methodologies. In some cases, introduction of a recombinant expression vector including a nucleic acid encoding Dlx2, or a functional fragment thereof, into a host glial cell in vitro or in vivo for expression of exogenous Dlx2 in the host glial cell to convert the glial cell to a neuron is accomplished by any of various transfection methodologies. In some cases, introduction of a recombinant expression vector including a nucleic acid encoding Isl1, or a functional fragment thereof, into a host glial cell in vitro or in vivo for expression of exogenous Isl1 in the host glial cell to convert the glial cell to a neuron is accomplished by any of various transfection methodologies.

Expression of (i) exogenous nucleic acid encoding any of the polypeptides, or any combination of polypeptides, described herein (e.g., NeuroD1, Isl1, Lhx3, Dlx2, and Ngn2) and/or (ii) exogenous nucleic acid encoding mir124 and mir218 in the host glial cell to convert the glial cell to a neuron is optionally achieved by introduction of mRNA encoding NeuroD1 (or any other polypeptide described herein), or a functional fragment thereof, to the host glial cell in vitro or in vivo.

Expression of (i) exogenous nucleic acid encoding any of the polypeptides, or any combination of polypeptides, described herein (e.g., NeuroD1, Isl1, Lhx3, Dlx2, and Ngn2) and/or (ii) exogenous nucleic acid encoding mir124 and mir218 in the host glial cell to convert the glial cell to a neuron is optionally achieved by introduction of polypeptides to the host glial cell in vitro or in vivo. Details of these and other techniques are known in the art, for example, as described in J. Sambrook and D. W. Russell, Molecular Cloning: A Laboratory Manual, Cold Spring Harbor Laboratory Press; 3rd Ed., 2001; F. M. Ausubel, Ed., Short Protocols in Molecular Biology, Current Protocols; 5th Ed., 2002; and Engelke, D. R., RNA Interference (RNAi): Nuts and Bolts of RNAi Technology, DNA Press LLC, Eagleville, PA, 2003.

An expression vector including (i) exogenous nucleic acid encoding any of the polypeptides, or any combination of polypeptides, described herein (e.g., NeuroD1, Isl1, Lhx3, Dlx2, and Ngn2) and/or (ii) exogenous nucleic acid encoding mir124 and mir218 or a functional fragment thereof, mRNA encoding the polypeptides or a functional fragment thereof, full-length or a functional fragment thereof, is optionally associated with a carrier for introduction into a host cell in vitro or in vivo.

In particular aspects, the carrier is a particulate carrier such as lipid particles including liposomes, micelles, unilamellar or mulitlamellar vesicles; polymer particles such as hydrogel particles, polyglycolic acid particles or polylactic acid particles; inorganic particles such as calcium phosphate particles such as described in for example U.S. Pat. No. 5,648,097; and inorganic/organic particulate carriers such as described for example in U.S. Pat. No. 6,630,486.

A particulate carrier can be selected from among a lipid particle; a polymer particle; an inorganic particle; and an inorganic/organic particle. A mixture of particle types can also be included as a particulate pharmaceutically acceptable carrier.

A particulate carrier is typically formulated such that particles have an average particle size in the range of about 1 nm-10 microns. In particular aspects, a particulate carrier is formulated such that particles have an average particle size in the range of about 1 nm-100 nm.

Further description of liposomes and methods relating to their preparation and use may be found in Liposomes: A Practical Approach (The Practical Approach Series, 264), V. P. Torchilin and V. Weissig (Eds.), Oxford University Press; 2nd ed., 2003. Further aspects of nanoparticles are described in S. M. Moghimi et al., FASEB J. 2005, 19, 311-30.

Expression of (i) exogenous nucleic acid encoding any of the polypeptides, or any combination of polypeptides, described herein (e.g., NeuroD1, Isl1, Lhx3, Dlx2, and Ngn2) and/or (ii) exogenous nucleic acid encoding mir124 and mir218 using a recombinant expression vector is accomplished by introduction of the expression vector into a eukaryotic or prokaryotic host cell expression system such as an insect cell, mammalian cell, yeast cell, bacterial cell or any other single or multicellular organism recognized in the art. Host cells are optionally primary cells or immortalized derivative cells. Immortalized cells are those which can be maintained in-vitro for at least 5 replication passages.

Host cells containing the recombinant expression vector are maintained under conditions wherein NeuroD1 is produced. In some cases, host cells containing the recombinant expression vector are maintained under conditions wherein Dlx2 is produced. Host cells may be cultured and maintained using known cell culture techniques such as described in Celis, Julio, ed., 1994, Cell Biology Laboratory Handbook, Academic Press, N.Y. Various culturing conditions for these cells, including media formulations with regard to specific nutrients, oxygen, tension, carbon dioxide and reduced serum levels, can be selected and optimized by one of skill in the art.

In some cases, a recombinant expression vector including (i) exogenous nucleic acid encoding any of the polypeptides, or any combination of polypeptides, described herein (e.g., NeuroD1, Isl1, Lhx3, Dlx2, and Ngn2) and/or (ii) exogenous nucleic acid encoding mir124 and mir218 is introduced into glial cells of a subject. Expression of (i) exogenous nucleic acid encoding any of the polypeptides, or any combination of polypeptides, described herein (e.g., NeuroD1, Isl1, Lhx3, and Ngn2) and/or (ii) exogenous nucleic acid encoding mir124 and mir218 in the glial cells “converts” the glial cells into neurons.

In some cases, a recombinant expression vector including (i) exogenous nucleic acid encoding any of the polypeptides, or any combination of polypeptides, described herein (e.g., NeuroD1, Isl1, Lhx3, Dlx2, and Ngn2) and/or (ii) exogenous nucleic acid encoding mir124 and mir218 is introduced into astrocytes of a subject. Expression of (i) exogenous nucleic acid encoding any of the polypeptides, or any combination of polypeptides, described herein (e.g., NeuroD1, Isl1, Lhx3, Dlx2, and Ngn2) and/or (ii) exogenous nucleic acid encoding mir124 and mir218 in the glial cells “converts” the astrocytes into neurons.

In some cases, a recombinant expression vector (i) exogenous nucleic acid encoding any of the polypeptides, or any combination of polypeptides, described herein (e.g., NeuroD1, Isl1, Lhx3, Dlx2, and Ngn2) and/or (ii) exogenous nucleic acid encoding mir124 and mir218 is introduced into reactive astrocytes of a subject. Expression of (i) exogenous nucleic acid encoding any of the polypeptides, or any combination of polypeptides, described herein (e.g., NeuroD1, Isl1, Lhx3, Dlx2, and Ngn2) and/or (ii) exogenous nucleic acid encoding mir124 and mir218 in the reactive astrocytes “converts” the reactive astrocytes into neurons.

In some cases, a recombinant expression vector including (i) exogenous nucleic acid encoding any of the polypeptides, or any combination of polypeptides, described herein (e.g., NeuroD1, Isl1, Lhx3, Dlx2, and Ngn2) and/or (ii) exogenous nucleic acid encoding mir124 and mir218 is introduced into NG2 cells of a subject. Expression of exogenous (i) exogenous nucleic acid encoding any of the polypeptides, or any combination of polypeptides, described herein (e.g., NeuroD1, Isl1, Lhx3, Dlx2, and Ngn2) and/or (ii) exogenous nucleic acid encoding mir124 and mir218 in the NG2 cells “converts” the NG2 cells into neurons.

Detection of expression of (i) exogenous nucleic acid encoding any of the polypeptides, or any combination of polypeptides, described herein (e.g., NeuroD1, Isl1, Lhx3, Dlx2, and Ngn2) and/or (ii) exogenous nucleic acid encoding mir124 and mir218 following introduction of a recombinant expression vector including a nucleic acid encoding (i) exogenous nucleic acid encoding any of the polypeptides, or any combination of polypeptides, described herein (e.g., NeuroD1, Isl1, Lhx3, Dlx2, and Ngn2) and/or (ii) exogenous nucleic acid encoding mir124 and mir218 is accomplished using any of various standard methodologies including, but not limited to, immunoassays, nucleic acid detection assays and detection of a reporter gene co-expressed with the exogenous nucleic acids.

The terms “converts” and “converted” are used herein to describe the effect of expression of NeuroD1 or a functional fragment thereof alone or in combination with Dlx2 or a functional fragment thereof resulting in a change of a glial cell, astrocyte or reactive astrocyte phenotype to a neuronal phenotype. Similarly, the phrases “NeuroD1 converted neurons,” “NeuroD1 and Dlx2 converted neurons” and “converted neurons” are used herein to designate a cell including exogenous NeuroD1 protein or a functional fragment thereof alone or in combination with exogenous Dlx2 protein or a functional fragment thereof which has consequent neuronal phenotype.

The term “phenotype” refers to well-known detectable characteristics of the cells referred to herein. The neuronal phenotype can be, but is not limited to, one or more of: neuronal morphology, expression of one or more neuronal markers, electrophysiological characteristics of neurons, synapse formation and release of neurotransmitter. For example, neuronal phenotype encompasses but is not limited to: characteristic morphological aspects of a neuron such as presence of dendrites, an axon and dendritic spines; characteristic neuronal protein expression and distribution, such as presence of synaptic proteins in synaptic puncta, presence of MAP2 in dendrites; and characteristic electrophysiological signs such as spontaneous and evoked synaptic events.

In a further example, glial phenotype such as astrocyte phenotype and reactive astrocyte phenotypes encompasses but is not limited to: characteristic morphological aspects of astrocytes and reactive astrocytes such as a generally “star-shaped” morphology; and characteristic astrocyte and reactive astrocyte protein expression, such as presence of glial fibrillary acidic protein (GFAP).

The term “nucleic acid” refers to RNA or DNA molecules having more than one nucleotide in any form including single-stranded, double-stranded, oligonucleotide or polynucleotide. The term “nucleotide sequence” refers to the ordering of nucleotides in an oligonucleotide or polynucleotide in a single-stranded form of nucleic acid.

The term “NeuroD1 nucleic acid” refers to an isolated NeuroD1 nucleic acid molecule and encompasses isolated NeuroD1 nucleic acids having a sequence that has at least 70%, 75%, 80%, 85%, 90%, 91%, 92%, 93%, 94%, 95%, 96%, 97%, 98%, or 99% identity to the DNA sequence set forth in SEQ ID NO:1 or SEQ ID NO:3, or the complement thereof, or a fragment thereof, or an isolated DNA molecule having a sequence that hybridizes under high stringency hybridization conditions to the nucleic acid set forth as SEQ ID NO:1 or SEQ ID NO:3, a complement thereof or a fragment thereof.

The nucleic acid of SEQ ID NO:3 is an example of an isolated DNA molecule having a sequence that hybridizes under high stringency hybridization conditions to the nucleic acid set forth in SEQ ID NO:1. A fragment of a NeuroD1 nucleic acid is any fragment of a NeuroD1 nucleic acid that is operable in an aspect described herein including a NeuroD1 nucleic acid.

A nucleic acid probe or primer able to hybridize to a target NeuroD1 mRNA or cDNA can be used for detecting and/or quantifying mRNA or cDNA encoding NeuroD1 protein. A nucleic acid probe can be an oligonucleotide of at least 10, 15, 30, 50 or 100 nucleotides in length and sufficient to specifically hybridize under stringent conditions to NeuroD1 mRNA or cDNA or complementary sequence thereof. A nucleic acid primer can be an oligonucleotide of at least 10, 15 or 20 nucleotides in length and sufficient to specifically hybridize under stringent conditions to the mRNA or cDNA, or complementary sequence thereof.

The terms “complement” and “complementary” refers to Watson-Crick base pairing between nucleotides and specifically refers to nucleotides hydrogen bonded to one another with thymine or uracil residues linked to adenine residues by two hydrogen bonds and cytosine and guanine residues linked by three hydrogen bonds. In general, a nucleic acid includes a nucleotide sequence described as having a “percent complementarity” to a specified second nucleotide sequence. For example, a nucleotide sequence may have 80%, 90%, or 100% complementarity to a specified second nucleotide sequence, indicating that 8 of 10, 9 of 10 or 10 of 10 nucleotides of a sequence are complementary to the specified second nucleotide sequence. For instance, the nucleotide sequence 3′-TCGA-5′ is 100% complementary to the nucleotide sequence 5′-AGCT-3′. Further, the nucleotide sequence 3′-TCGA- is 100% complementary to a region of the nucleotide sequence 5′-TTAGCTGG-3′.

The terms “hybridization” and “hybridizes” refer to pairing and binding of complementary nucleic acids. Hybridization occurs to varying extents between two nucleic acids depending on factors such as the degree of complementarity of the nucleic acids, the melting temperature, Tm, of the nucleic acids and the stringency of hybridization conditions, as is well known in the art. The term “stringency of hybridization conditions” refers to conditions of temperature, ionic strength, and composition of a hybridization medium with respect to particular common additives such as formamide and Denhardt's solution.

Determination of particular hybridization conditions relating to a specified nucleic acid is routine and is well known in the art, for instance, as described in J. Sambrook and D. W. Russell, Molecular Cloning: A Laboratory Manual, Cold Spring Harbor Laboratory Press; 3rd Ed., 2001; and F. M. Ausubel, Ed., Short Protocols in Molecular Biology, Current Protocols; 5th Ed., 2002. High stringency hybridization conditions are those which only allow hybridization of substantially complementary nucleic acids. Typically, nucleic acids having about 85-100% complementarity are considered highly complementary and hybridize under high stringency conditions. Intermediate stringency conditions are exemplified by conditions under which nucleic acids having intermediate complementarity, about 50-84% complementarity, as well as those having a high degree of complementarity, hybridize. In contrast, low stringency hybridization conditions are those in which nucleic acids having a low degree of complementarity hybridize.

The terms “specific hybridization” and “specifically hybridizes” refer to hybridization of a particular nucleic acid to a target nucleic acid without substantial hybridization to nucleic acids other than the target nucleic acid in a sample.

Stringency of hybridization and washing conditions depends on several factors, including the Tm of the probe and target and ionic strength of the hybridization and wash conditions, as is well-known to the skilled artisan. Hybridization and conditions to achieve a desired hybridization stringency are described, for example, in Sambrook et al., Molecular Cloning: A Laboratory Manual, Cold Spring Harbor Laboratory Press, 2001; and Ausubel, F. et al., (Eds.), Short Protocols in Molecular Biology, Wiley, 2002.

An example of high stringency hybridization conditions is hybridization of nucleic acids over about 100 nucleotides in length in a solution containing 6×SSC, 5×Denhardt's solution, 30% formamide, and 100 micrograms/ml denatured salmon sperm at 37° C. overnight followed by washing in a solution of 0.1×SSC and 0.1% SDS at 60° C. for 15 minutes. SSC is 0.15M NaCl/0.015M Na citrate. Denhardt's solution is 0.02% bovine serum albumin/0.02% FICOLL/0.02% polyvinylpyrrolidone. Under highly stringent conditions, SEQ ID NO:1 and SEQ ID NO:3 will hybridize to the complement of substantially identical targets and not to unrelated sequences.

Methods of treating a neurological condition in a subject in need thereof are provided according to aspects of the disclosure which include delivering a therapeutically effective (i) exogenous nucleic acid encoding any of the polypeptides, or any combination of polypeptides, described herein (e.g., NeuroD1, Isl1, Lhx3, Dlx2, and Ngn2) and/or (ii) exogenous nucleic acid encoding mir124 and mir218 to glial cells of the central nervous system or peripheral nervous system of the subject, the therapeutically effective amount of (i) exogenous nucleic acid encoding any of the polypeptides, or any combination of polypeptides, described herein (e.g., NeuroD1, Isl1, Lhx3, Dlx2, and Ngn2) and/or (ii) exogenous nucleic acid encoding mir124 and mir218 in the glial cells results in a greater number of neurons in the subject compared to an untreated subject having the same neurological condition, whereby the neurological condition is treated.

The conversion of reactive glial cells into neurons also reduces neuroinflammation and neuroinhibitory factors associated with reactive glial cells, thereby making the glial scar tissue more permissive to neuronal growth so that neurological condition is alleviated.

The term “neurological condition” or “neurological disorder” as used herein refers to any condition of the central nervous system of a subject which is alleviated, ameliorated or prevented by additional neurons. Injuries or diseases which result in loss or inhibition of neurons and/or loss or inhibition of neuronal function are neurological conditions for treatment by methods described herein.

Injuries or diseases which result in loss or inhibition of glutamatergic neurons and/or loss or inhibition of glutaminergic neuronal functions are neurological conditions that can be treated as described herein. Loss or inhibition of other types of neurons, such as GABAergic, cholinergic, dopaminergic, norepinephrinergic, or serotonergic neurons can be treated with the similar method.

The term “therapeutically effective amount” as used herein is intended to mean an amount of an inventive composition which is effective to alleviate, ameliorate or prevent a symptom or sign of a neurological condition to be treated. In particular embodiments, a therapeutically effective amount is an amount which has a beneficial effect in a subject having signs and/or symptoms of a neurological condition.

The terms “treat,” “treatment,” “treating,” “NeuroD1 treatment,” and “NeuroD1 and Dlx2 treatment” or grammatical equivalents as used herein refer to alleviating, inhibiting or ameliorating a neurological condition, symptoms or signs of a neurological condition, and preventing symptoms or signs of a neurological condition, and include, but are not limited to therapeutic and/or prophylactic treatments.

Signs and symptoms of neurological conditions are well-known in the art along with methods of detection and assessment of such signs and symptoms.

In some cases, combinations of therapies for a neurological condition of a subject can be administered.

According to particular aspects an additional pharmaceutical agent or therapeutic treatment administered to a subject to treats the effects of disruption of normal blood flow in the CNS in an individual subject in need thereof include treatments such as, but not limited to, removing a blood clot, promoting blood flow, administration of one or more anti-inflammation agents, administration of one or more anti-oxidant agents, and administration of one or more agents effective to reduce excitotoxicity

The term “subject” refers to humans and also to non-human mammals such as, but not limited to, non-human primates, cats, dogs, sheep, goats, horses, cows, pigs and rodents, such as but not limited to, mice and rats; as well as to non-mammalian animals such as, but not limited to, birds, poultry, reptiles, amphibians.

Embodiments of inventive compositions and methods are illustrated in the following examples. These examples are provided for illustrative purposes and are not considered limitations on the scope of inventive compositions and methods.

### EXAMPLES

### Materials and Methods

### Animal Use

GAD-GFP mice (Tg[Gad1-EGFP]94Agmo/J) and wild-type C57BL/6 mice were purchased from the Jackson Laboratory and bred in house. Mice of 2-4 months old (both male and female) were used. Mice were housed in a 12 hour light/dark cycle and supplied with sufficient food and water. All animal use and studies were approved by the Institutional Animal Care and Use Committee (IACUC) of the Pennsylvania State University. All procedures were carried out in accordance with the approved protocols and guidelines of National Institute of Health (NIH).

### Retrovirus and AAV Production

Retroviral vectors expressing GFP and NeuroD1-GFP under the CAG promoter (pCAG) were previously described (Guo et al., Cell Stem Cell, 14:188-202 (2014)). Retrovirus packaging, purification and titering were performed as previously described (Guo et al., Cell Stem Cell, 14:188-202 (2014)). For AAV-mediated gene expression, the Cre-Flex system was applied to target transgene expression specifically to reactive astrocytes using the human GFAP (hGFAP) promoter. To generate pAAV-hGFAP::Cre vector, the hGFAP promoter was first amplified from pDRIVE-hGFAP plasmid (InvivoGen) by PCR and inserted into pAAV-MCS (Cell Biolab) between the MluI and SacII sites to replace the CMV promoter. The Cre gene coding fragment was then similarly subcloned from phGFAP-Cre (Addgene plasmid #40591) and inserted into pAAV MCS between the EcoRI and SalI sites. To construct pAAV-FLEX vectors expressing transgenes, the coding sequences of NeuroD1, mCherry or GFP were amplified by PCR from the corresponding retroviral constructs. The NeuroD1 fragment was fused with either P2A-mCherry or P2A-GFP and subcloned into the pAAV-FLEX-GFP vector (Addgene plasmid #28304) between the KpnI and XhoI sites. All plasmid constructs were confirmed by sequencing. The AAV-CamKII-GFP plasmid was purchased from Addgene (#64545). For AAV production, HEK 293T cells were transfected with the pAAV expression vectors, pAAV9-RC vector (Cell Biolab), and pHelper vector (Cell Biolab) to generate AAV particles carrying our transgenes. Three days after transfection, the cells were scraped in their medium and centrifuged. The supernatant was then discarded and the cell pellet was frozen and thawed four times, resuspended in a discontinuous iodixanol gradient, and centrifuged at 54,000 rpm for two hours. Finally, the virus-containing layer was extracted, and the viruses were concentrated using Millipore Amicon Ultra Centrifugal Filters. The viral titers were determined using the QuickTiter™ AAV Quantitation Kit (Cell Biolabs) and then diluted to a final concentration of 1×10¹⁰ genome copy (GC)/mL for injection.

### Laminectomy, Injury, and Stereotaxic Viral Injection

Mice were anesthetized by intraperitoneal injection of ketamine/xylazine (80-120 mg/kg ketamine; 10-16 mg/kg xylazine). A laminectomy was then performed at the T11-T12 vertebrae to expose dorsal surface of the spinal cord, and a stab or contusion injury was performed. The stab injury was conducted with a 31-gauge needle at the center of the exposed surface, 0.4 mm lateral to the central artery with a depth of 0.4 mm, while the contusion injury was generated with a force of 45 kdyn on an Infinite Horizon Impactor (IH-0400, Precision Systems and Instrumentation) directly at the center of the exposed surface. Either immediately following the injury or at a specified delay, 1.0 μL of concentrated virus was injected using a 50 μL Hamilton syringe with a 34-gauge injection needle at a rate of 0.05 μL/minute at the same coordinates for the stab injury or 1 mm away for the contusion injury. The needle was then kept in place for three minutes after injection to prevent drawing out the virus during withdrawal. The surgical area was then treated with antibiotic ointment, and the skin was clipped for a week to allow the skin to re-suture. The mice were kept on a heating pad and treated with Carprofen for pain relieve via subcutaneous injection (5 mg/kg) on the day of surgery and drinking water (10 mg/kg) for three days after surgery and closely monitored for one week to ensure full recovery of health.

### Electrophysiology

Mice were sacrificed at defined time points by anesthetization with 2.5% Avertin and decapitation. The spinal cord segment was then removed from the spine into cutting solution (125 mM NaCl, 2.5 mM KCl, 1.3 mM MgSO₄, 26 mM NaHCO₃, 1.25 mM NaH₂PO₄, 2.0 mM CaCl₂ and 10 mM glucose adjusted to pH 7.4 and 295 mOsm/L and bubbled for one hour with 95% O₂/5% CO₂) cooled on ice, where it was encased in an agarose matrix (Sigma) and cut into 300 μm thickness slices using a VT3000 vibratome (Leica). Slices were then incubated for one hour in holding solution (92 mM NaCl, 2.5 mM KCl, 1.25 mM NaH₂PO₄, 30 mM NaHCO₃, 20 mM HEPES, 15 mM glucose, 12 mM N-Acetyl-L-cysteine, 5 mM Sodium ascorbate, 2 mM Thiourea, 3 mM Sodium pyruvate, 2 mM MgSO₄, and 2 mM CaCl₂, adjusted to pH 7.4 and 295 mOsm/L and bubbled continuously with 95% O₂/5% CO₂) at room temperature before patch-clamp recording in standard ACSF (125 mM NaCl, 2.5 mM KCl, 1.25 mM NaH₂PO₄, 26 mM NaHCO₃, 1.3 MgSO₄, 2.5 mM CaCl₂, and 10 mM glucose adjusted to pH 7.4 and 295 mOsm/L and bubbled for one hour with 95% O₂/5% CO₂). Both native and converted cells were recorded by whole-cell recording using standard inner solution (135 mM K-gluconate, 10 mM KCl, 5 mM Naphosphocreatine, 10 mM HEPES, 2 mM EGTA, 4 mM MgATP, and 0.5 mM Na₂GTP, adjusted to pH 7.4 and 295 mOsm/L) with the membrane potential held at −70 mV. Typical values for the pipette and total series resistances were 2-10 MQ and 20-60 MQ, respectively. Data were collected using the pClamp 9 software (Molecular Devices) by sampling at 10 kHz and filtering at 1 kHz. Data were then analyzed and plotted with the Clampfit 9.0 software (Molecular Devices).

### Immunohistochemistry, Immunocytochemistry, and Microscopy

After perfusion, the target region of the spinal cord (˜0.5 cm in length) was surgically dissected, fixed in 4% paraformaldehyde (PFA) in PBS for one day, dehydrated in 30% sucrose solution for one day, and sectioned into 30 μm coronal or horizontal slices using a Leica CM1950 cryostat. The slices were collected serially in 24-well plates so that distance from the injury site could later be ascertained. The samples were then stored at 4° C. in 0.02% sodium azide (NaN₃) in PBS to prevent bacterial degradation. Spinal cord slices were chosen for immunohistochemistry based on infection of the dorsal horn by inspecting the reporter protein (mCherry or GFP) in the storage solution under a fluorescent microscope. For the stab injury experiments, care was taken to select coronal slices at least 100 μm from the injury site to ensure tissue integrity. On the first day of staining, samples were washed in PBS three times for five minutes per wash, permeablized with 2% Triton X-100 in PBS for 20 minutes, and blocked using a 5% normal donkey serum (NDS) and 0.1% Triton-X in PBS for two hours to reduce non-specific binding of the antibodies. The samples were then incubated with primary antibodies diluted in the same blocking buffer at 4° C. for two nights to allow thorough penetration of the antibodies. On the third day, the samples were recovered to room temperature, washed in PBS three times for five minutes per wash, and incubated with secondary antibodies diluted in blocking buffer for one hour. Finally, the samples were washed in PBS three more times for ten minutes per wash and mounted on glass slides with coverslips using anti-fading mounting solution (Invitrogen). The immunostained samples were examined and imaged using Olympus FV1200 and Zeiss LSM 800 laser confocal microscopes. Z-stacks were collected for the in vivo images for the whole thickness of the samples, and maximum intensity and z-stack projections were used for image preparation and analysis.

### Quantification and Data Analysis

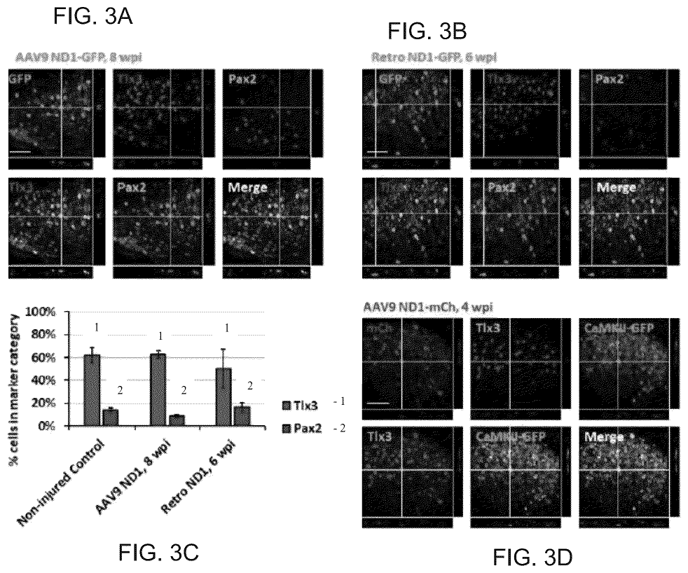

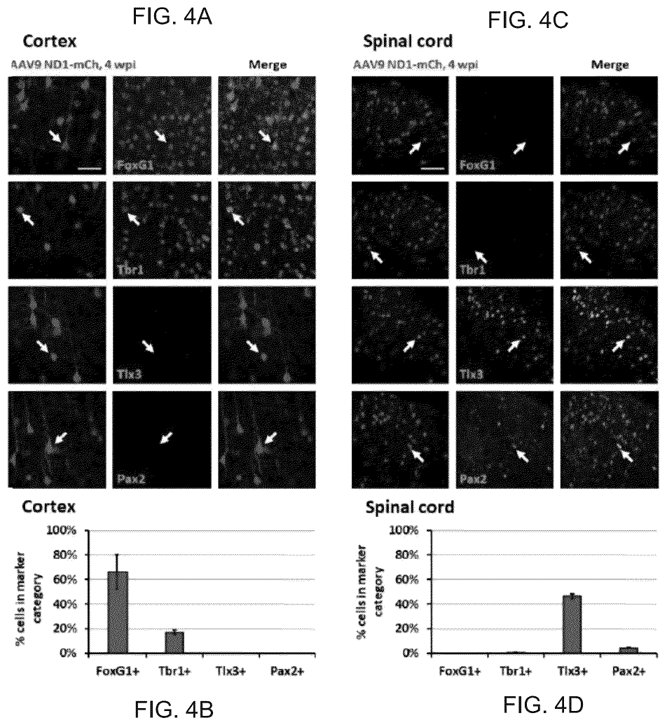

As a result of our carefully selected injection coordinates described above, infected cells were mostly found in the dorsal horn of the spinal cord, Rexed laminae 1-6 (Rexed, J. Comp. Neurol., 100:297-379 (1954)). For most of the quantification, including cell conversion and NeuN acquisition, cells were counted if they appeared in any part of this region. For quantification based on cell subtype (FIGS. 3 and 4), cells were only counted if they appeared in Rexed laminae 1-3, centered about the substantia gelatinosa, a region dominated by small, excitatory interneurons and easily demarcated due to its high cell density (Santos et al., J. Physiol., 581:241-254 (2007)). This region was chosen for its ease of demarcation and so that a consistent local population of neuronal subtypes could be expected from sample to sample. Quantification was performed on collected images using the z-stacked images as a guide and the layered stacks to check the vertical dimension. Strict background cutoffs for positive signals were calculated for each channel as three times the average background intensity for the relevant tissue and antibody. Cells were binned by presence (i.e., above the background cutoff) or absence (i.e., below the background cutoff) for each marker in question, using the viral fluorophore (mCherry or GFP) to identify infected cells and DAPI to confirm each cell for counting. To estimate the total number of converted neurons per infection for our contusion experiments, we multiplied the average number of NeuN+ infected cells per horizontal section, calculated from one dorsal, one central, and one ventral section, by the total number of horizontal sections per sample. All quantification was performed on three biological replicates per data point and is reported as the means and standard deviations of the three replicates.

### Example 1—NeuroD1 Reprograms Reactive Astrocytes into Neurons in the Injured Spinal Cord

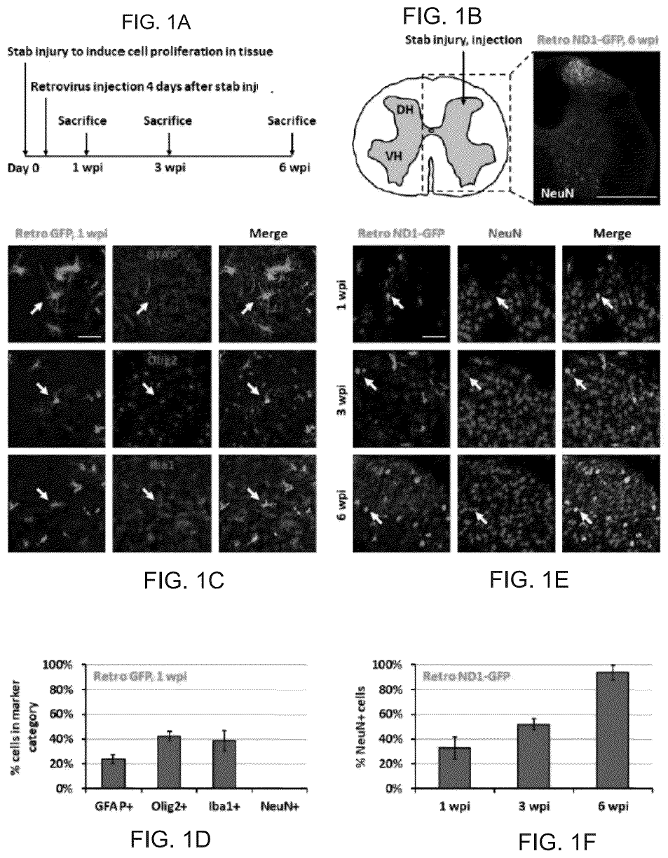

SCI has been studied for decades but so far there is still limited therapy to treat SCI patients. Besides axonal degeneration, neuronal loss following SCI is a major obstacle for functional recovery. We previously demonstrated that expressing NeuroD1 in reactive astrocytes after brain injury can directly convert astrocytes into neurons (Guo et al., Cell Stem Cell, 14:188-202 (2014)). In this study, we investigated whether such in vivo direct conversion technology can be used to regenerate functional new neurons in injured spinal cord. To target the dividing reactive astrocytes after injury, we employed retroviruses that mainly express ectopic genes in dividing cells but not in neurons, which cannot divide. We injected NeuroD1-expressing retroviruses at 4 days post-stab injury (dpi), when many dividing reactive astrocytes have been detected (Chen et al., J. Neurosci., 28:10983-10989 (2008); and Hong et al., Glia, 62:2044-2060 (2014)), and analyzed samples at 1, 3, and 6 weeks-post-injection (wpi) (FIG. 1A). In this study, we chose the spinal cord dorsal horn as our major region of interest because it is composed of both excitatory and inhibitory neurons and is critical to afferent sensory information processing (FIG. 1B). Motor neuron regeneration in the spinal cord ventral horn was studied separately. We first explored the cell types infected by our control CAG::GFP retroviruses. At 1 wpi, we found that the control GFP retroviruses infected a mixture of glial cell types including reactive astrocytes (GFAP+ and some GFAP+/Olig2+), oligodendrocyte progenitor cells (OPCs) (Olig2+), and microglia (Iba1+) (FIGS. 1C and 1D), but not NeuN+ neurons (FIG. 1D). In contrast, cells infected by the CAG::NeuroD1-GFP retrovirus showed an increasing number of NeuN+ cells with neuronal morphology over time (FIG. 1E), and quantitatively reached 93.5%, which is a high efficiency, at 6 wpi (FIG. 1F), indicating a successful glia-to-neuron conversion in the injured spinal cord.

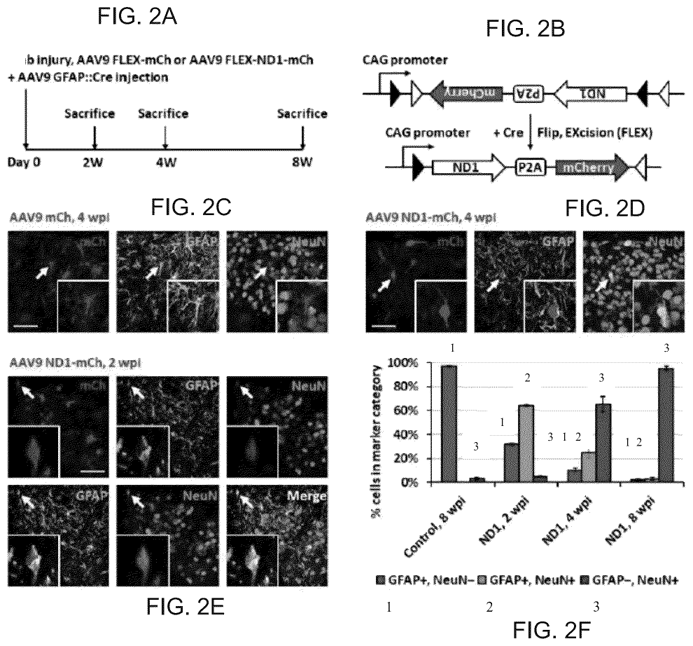

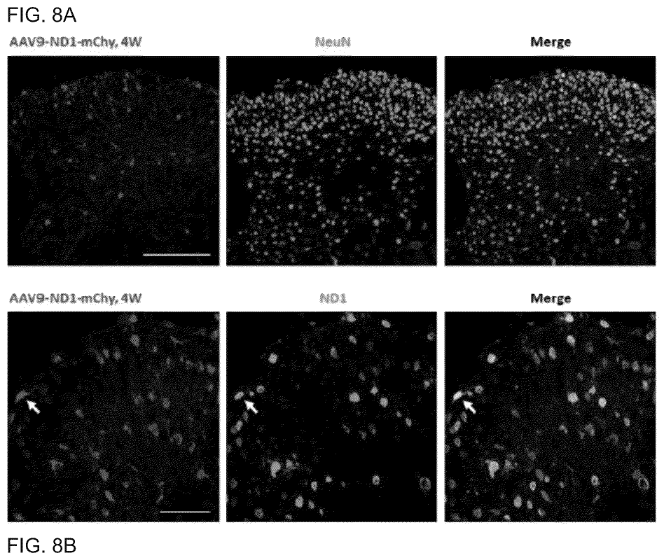

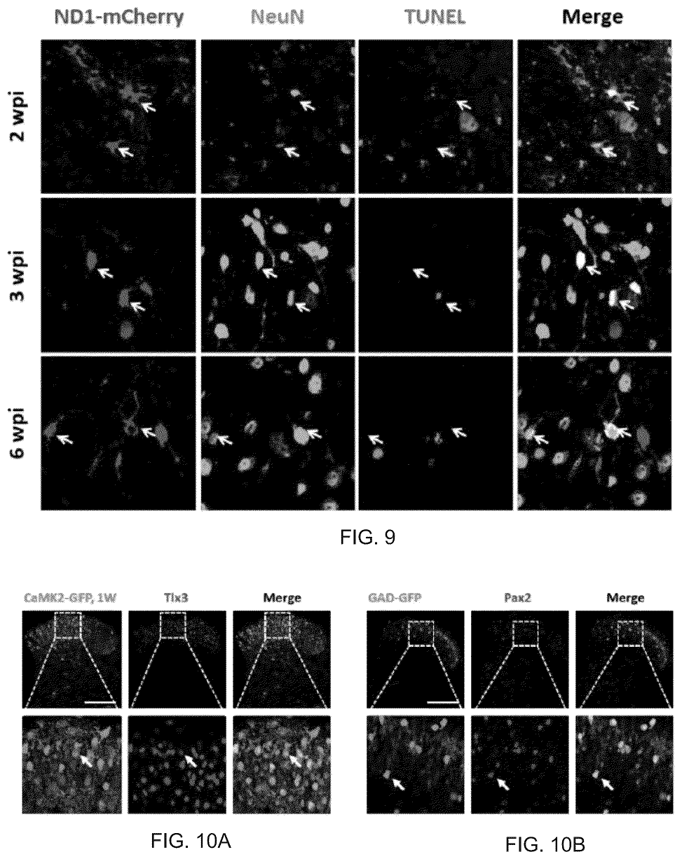

While retroviruses can target reactive glial cells, the number of dividing glial cells at the time of viral infection may be limited. We adopted an AAV gene delivery system in which the transgene expression is controlled by an astrocyte-specific GFAP promoter (FIG. 2A). Specifically, we used a Cre-Flex gene expression system, which contains two AAV vectors, with one encoding GFAP-Cre and the other encoding the transgene in reverse form flanked by double LoxP sites (FLEX vector) (Atasoy et al., J. Neurosci., 28:7025-7030 (2008); Chen et al., BioRxiv, Apr. 4, 2018; doi: http://dx.doi.org/10.1101/294967); and Liu et al., J. Neurosci., 35:9336-9355 (2015)). Thus, when the two AAVs are co-injected into the spinal cord, Cre recombinase will be expressed in the infected reactive astrocytes and turn on the transgene expression in FLEX vector by flipping the transgene sequence into the correct form for transcription (FIG. 2B). We first confirmed the specificity of the Cre-Flex AAV system in the spinal cord by co-injecting AAV GFAP::Cre and AAV FLEXCAG::mCherry (or::GFP) into the stab-injured dorsal horn. The control virus infected cells were mostly GFAP+, NeuN-astrocytes at 4 wpi (FIG. 2C). Next, we co-injected AAV GFAP::Cre with AAV FLEX-CAG::NeuroD1-P2A-mCherry into the stab-injured dorsal horn. In contrast to the control AAV, the NeuroD1-mCherry infected cells were mostly NeuN+/GFAP-neurons with clear neuronal morphology at 4 wpi (FIG. 2D). NeuroD1 overexpression in the infected cells was confirmed by immunostaining (FIG. 8). Interestingly, besides NeuN+/GFAP-converted neurons, we also observed many NeuroD1-AAV-infected cells at 2 wpi with co-immunostaining of both GFAP+ and NeuN+(FIG. 2E), suggesting a potential intermediate stage during astrocyteto-neuron conversion. We termed these GFAP+/NeuN+ cells induced by NeuroD1 expression in astrocytes as “AtN transitional cells”. We did not observe any such transitional cells in the control mCherry-infected spinal cord after injury, suggesting that AtN conversion does not happen following neural injury but can be induced by ectopic expression of transcription factors such as NeuroD1. Quantitative analysis revealed that the control AAV-infected cells were mostly GFAP+ astrocytes by 8 wpi (FIG. 2F, left bars), whereas NeuroD1 AAV-infected cells showed a progressive increase in the percentage of neurons (NeuN+/GFAP-, right bars in FIG. 2F) from 2 to 8 wpi, reaching ˜95% at 8 wpi (FIG. 2F, far right bar). Note that at 2 wpi, over 60% of NeuroD1-infected cells were GFAP+/NeuN+ transitional cells (middle bar in FIG. 2F), which gradually decreased at 4 wpi and 8 wpi together with a decrease of GFAP+ astrocytes (left bars in FIG. 2F) among the NeuroD1-infected cell population. Further analysis showed that neither transitional cells nor converted neurons exhibited significant cell death suggesting that apoptosis does not play a significant role during the NeuroD1-mediated cell conversion process (FIG. 9). Comparing to Ngn2-mediated or Ascl1-mediated AtN conversion (Gascon et al., Cell Stem Cell, 18:396-409 (2016)), less apoptosis was detected during NeuroD1-mediated conversion process, which may suggest that different transcription factors act through different signaling and metabolic pathways to carry out cell conversion.

### Example 2—NeuroD1 Converts Dorsal Spinal Astrocytes Into Tlx3+ Glutamatergic Neurons

After demonstrating astrocyte-to-neuron conversion in the spinal cord, we next investigated which subtypes of neurons were generated through NeuroD1-mediated conversion. The dorsal horn of the spinal cord contains two main neuronal subtypes: glutamatergic and GABAergic neurons (Abraira and Ginty, Neuron, 79:618-639 (2013)). During spinal cord development, two transcription factors, Tlx3 and Pax2, appear to play roles in determining cell fate specification in the dorsal horn (Cheng et al., Nature Neurosci., 8:1510-1515 (2005); and Huang et al., Dev. Biol., 322:394-405 (2008)). Interestingly, by examining AAV NeuroD1-GFP infected cells in the dorsal horn at 8 wpi, we found that the majority of NeuroD1-converted neurons were Tlx3+(62.6±3.3%), suggesting a majority glutamatergic neuronal subtype (FIG. 3A). In contrast, only a small percentage of NeuroD1-converted neurons in the dorsal horn were Pax2+(8.8±1.3%), suggesting a minority GABAergic neuronal subtype (FIG. 3A). Because AAV might infect a small proportion of neurons (FIG. 2F, control), we further examined retrovirus NeuroD1-GFP-infected cells in the dorsal horn at 6 wpi and found that, similar to our AAV experiments, the retrovirus NeuroD1-converted neurons were mainly Tlx3+(FIG. 3B). As a control, we quantified the proportion of Tlx3+ and Pax2+ neurons in the spinal cord dorsal horn of uninjured and untreated mice, and found that the native proportions of Tlx3+ and Pax2+ cells were 62.0±6.4% and 14.2±2.0%, respectively (FIG. 3C, left two bars). In comparison, the proportion of Tlx3+ and Pax2+ neurons among the AAV NeuroD1-converted neurons were 62.6±3.3% and 8.8±1.3%, respectively (FIG. 3C, middle two bars); and among the retrovirus NeuroD1-converted neurons were 50.3±17.0% of Tlx3+ and 16.4±4.3% Pax2+ (FIG. 3C, right two bars). These results suggest that the majority of NeuroD1-converted neurons in the dorsal horn of spinal cord are Tlx3+ neurons, with a small proportion being Pax2+ neurons.

We further confirmed the neuronal subtypes after NeuroD1 conversion using AAV CaMKII-GFP to identify glutamatergic neurons and GAD-GFP transgenic mice to identify GABAergic neurons. When co-injecting AAV GFAP::Cre and Flex-NeuroD1-mCherry together with AAV CaMKII::GFP (Dittgen et al., PNAS, 101:18206-18211 (2004)), we observed 89.5±5.2% (n=3) of GFP+ cells coexpressing Tlx3+, confirming that these Tlx3+ neurons are indeed glutamatergic (FIG. 3D). Many NeuroD1-mCherry converted neurons were also colocalizing with CaMKII-GFP (FIG. 3D), suggesting that they were glutamatergic neurons. When injecting AAV GFAP::Cre and Flex-NeuroD1-mCherry in GAD-GFP mice (n=3), in which GABAergic neurons are genetically labeled with GFP, we did not observe GAD-GFP co-expression with Tlx3+, as expected. Indeed, the CaMKII and GAD markers co-stained consistently with endogenous Tlx3+ and Pax2+ neurons as well, which we observed in the dorsal horn of spinal cord in uninjured, untreated mice (FIG. 10). Therefore, the majority of NeuroD1-converted neurons in the dorsal horn of the spinal cord are glutamatergic neurons, consistent with our finding in the mouse cortex (Chen et al., BioRxiv, Apr. 4, 2018; doi: http://dx.doi.org/10.1101/294967).

### Example 3—NeuroD1-Converted Neurons Express Region-Specific Neuronal Subtype Markers

While NeuroD1-converted neurons appear to be mainly glutamatergic neurons in both the mouse cortex and spinal cord, we further investigated whether they are the same type of glutamatergic neurons or not. For this purpose, we injected the same AAV GFAP::Cre and AAV FLEX-NeuroD1-mCherry into the mouse M1 motor cortex and the spinal cord, and then performed a serial immunostaining using both cortical neuronal markers (FoxG1 and Tbr1) and spinal neuronal markers (Tlx3 and Pax2) at 4 wpi (FIG. 4). The majority of NeuroD1-infected cells were converted into neurons in both the brain and the spinal cord at 4 wpi (FIGS. 4A and 4C). Strikingly, when we compared the neuronal subtypes resulting from NeuroD1-mediated conversion in the brain versus the spinal cord side-by-side, a distinct pattern emerged: the converted neurons in the mouse cortex acquired cortical neuron markers such as FoxG1 (66.1±14.3%) and Tbr1 (17.1±1.9%), but not spinal neuron markers such as Tlx3 (0%) or Pax2 (0%) (FIGS. 4A and 4B). In contrast, the converted neurons in the spinal cord acquired spinal neuron markers Tlx3 (46.4±2.2%) and Pax2 (4.2±0.3%), but not cortical neuron markers FoxG1 (0%) or Tbr1 (0.6±0.5%) (FIGS. 4C and 4D). Morphologically, the converted neurons in the brain resembled cortical pyramidal neurons with larger cell bodies (FIG. 4A), while those in the spinal cord resembled dorsal horn interneurons with smaller cell bodies (FIG. 4C). The relative lower percentage of Tbr1+ cells among the converted neurons in the cortex suggest that the newly converted neurons may not be mature enough at 4 wpi and may take longer time to fully acquire their neuronal identity. These distinct differences in the neuronal identity after conversion by the same transcription factor in the brain versus the spinal cord suggest that the glial cell lineage, here cortical lineage versus spinal lineage, as well as the local environment may exert an important influence on the resulting subtypes of converted neurons.

### Example 4—NeuroD1-Converted Neurons are Physiologically Functional

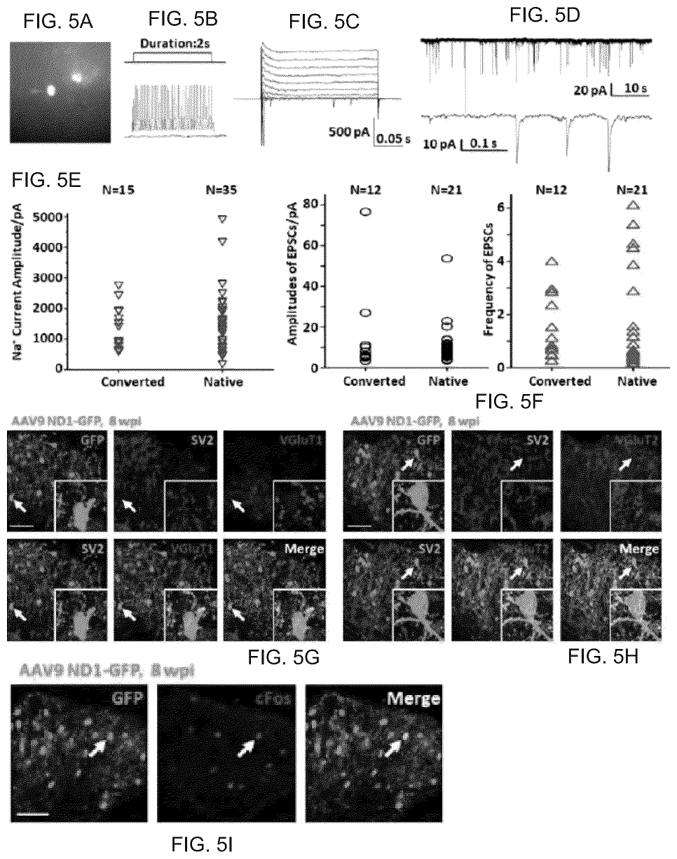

To test the functionality and circuit-integration of NeuroD1-converted neurons, we performed patch-clamp electrophysiological recordings of native and converted neurons on spinal cord slices from mice sacrificed at 8-10 wpi (FIG. 5A). The converted neurons could generate repetitive action potentials (FIG. 5B) and displayed large Na+ and K+ currents (FIG. 5C). Moreover, we detected robust spontaneous EPSCs from the NeuroD1-converted neurons (FIG. 5D). Quantitatively, we found that the NeuroD1-converted neurons showed similar levels of Na+ currents (FIG. 5E) and spontaneous EPSCs to their neighboring native neurons (FIG. 5F). Immunostaining with a series of synaptic markers including SV2 and VGlut1/VGlut2 further confirmed that the NeuroD1-converted neurons were surrounded by numerous synaptic puncta with many of them directly innervating the neuronal soma and dendrites (FIGS. 5G and 5H, cyan and yellow dots). Finally, cFos, an immediate early gene that is typically activated by neuronal activity during functional tasks, was clearly detected in some of the NeuroD1-converted neurons, indicating that they were functionally active in the local spinal cord circuits (FIG. 5I). These results demonstrate that NeuroD1 can reprogram reactive astrocytes into functional neurons in the dorsal horn of the injured spinal cord.

### Example 5—NeuroD1-Mediated Cell Conversion in the Contusive SCI Model

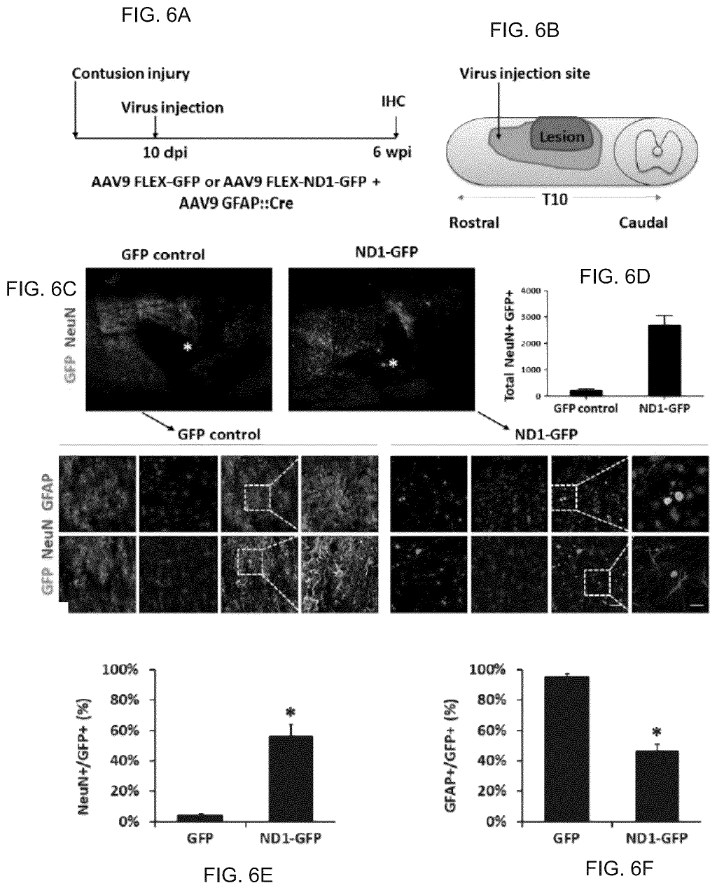

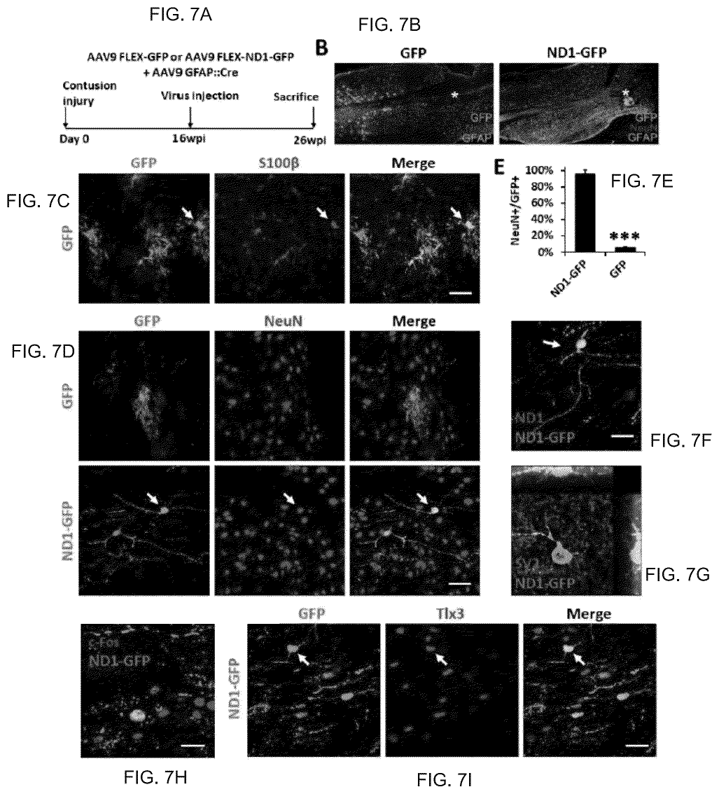

To move closer towards clinical situations, we evaluated NeuroD1-mediated neuronal conversion in the contusive SCI model. Compared with stab injury, contusive injury (e.g., a contusive injury created using a piston driven metal bar to strike the area) creates a much more severe injury environment, which could affect the efficiency of neuronal conversion and the survival of converted neurons. We therefore performed two experiments to test our AAV GFAP::Cre and Flex-NeuroD1-GFP system after contusive SCI: one short-delay injection to test our treatment as a response to acute injury (FIG. 6), and one long-delay injection to test our treatment as a response to chronic injury (FIG. 7). The advantage of the short-delay experiment is to maximize infection rate by taking advantage of the post-injury proliferation of reactive astrocytes, while the advantage of the long-delay experiment is to maximize the neuronal survival after conversion by allowing injury-induced neuroinflammation to taper down and minimize the secondary effects of the contusion injury. In our short-delay experiment, viral injection was conducted at 10 days post-contusive injury, and tissues were collected at 6 weeks post-viral infection (FIG. 6A). Viral injections were performed 1 mm away from the contusion site to avoid the injury core (FIG. 6B). The injury core is apparent after contusion and is characterized by the loss of NeuN+ neuronal cell bodies (FIG. 6C, labeled by *). Viral injection at 10 days post-contusion resulted in many GFP+ cells surrounding the injury core in both control GFP and NeuroD1-GFP groups (FIG. 6C), indicating good infection rate and survival of the AAV infected cells in the contusive SCI model. On the other hand, the AAV NeuroD1-GFP infected cells showed a dramatic morphological difference from the control GFP group (FIG. 6C). As illustrated in the enlarged images in FIG. 6C, the GFP infected cells in the control group showed typical astrocytic morphology and colocalization with GFAP signal (which stained magenta), but rarely showed any colocalization with the neuronal marker NeuN (which stained red). In contrast, NeuroD1-GFP infected cells were often colocalized with NeuN but rarely colocalized with GFAP (FIG. 6C), indicating successful neuronal conversion. Quantitatively, we counted the total number of converted neurons to be ˜2,600 cells surrounding the lesion core areas (FIG. 6D). The efficiency of NeuroD1-mediated neuronal conversion in the short-delay experiment as measured by NeuN immunoreactivity was ˜55% (FIG. 6E), while the remaining cells were mostly GFAP+(FIG. 6F). In contrast, the GFP-infected cells were mostly GFAP+ astrocytes and rarely NeuN+ neurons (only 3.9% NeuN+ in GFP group) (FIGS. 6E and 6F).

In our long-delay experiment, viral injection was conducted at 4 months post-contusive injury, when glial scar has been well formed after contusion, and tissues were collected at 10 weeks post-viral infection (FIG. 7A). FIG. 7B illustrates the overall morphology of the spinal cord with immunostaining of GFP, NeuN, and GFAP. As in the short-delay experiment, the lesion core (labeled by *) also lacked NeuN+ neurons. In the control AAV GFP alone group, the viral infected cells were mainly S100b+ astrocytes (FIG. 7C), but rarely showed any NeuN+ signal (FIG. 7D, top row). In contrast, the majority of NeuroD1-GFP infected cells were converted into NeuN+ neurons (FIG. 7D, bottom row; quantified in FIG. 7E). The NeuroD1-mediated conversion efficiency reached >95%, a very high efficiency (FIG. 7E). Immunostaining confirmed the NeuroD1 overexpression in the NeuroD1-GFP infected cells (FIG. 7F). Furthermore, NeuroD1-converted neurons at 10 wpi were surrounded by many synaptic puncta (SV2) with some of them directly innervating the soma and dendrites (FIG. 7G, yellow dots). We also identified c-Fos+ cells among NeuroD1-converted neurons (FIG. 7H), indicating that they were able to integrate into the local spinal cord functional circuitry. Lastly, some of the NeuroD1-converted neurons in the contusive SCI model at 10 wpi showed glutamatergic subtype through expression of Tlx3 in the dorsal horn (FIG. 7I), consistent with our stab injury model. Altogether, these results indicate that NeuroD1 overexpression can reprogram reactive astrocytes into functional neurons after contusive SCI under both acute and chronic treatment conditions, with higher conversion efficiency achieved after glial scar formation. This clinically relevant model can be used in future studies to further test functional improvement after SCI using in vivo cell conversion technology.

### Example 6—Astrocyte to Neuron Conversion

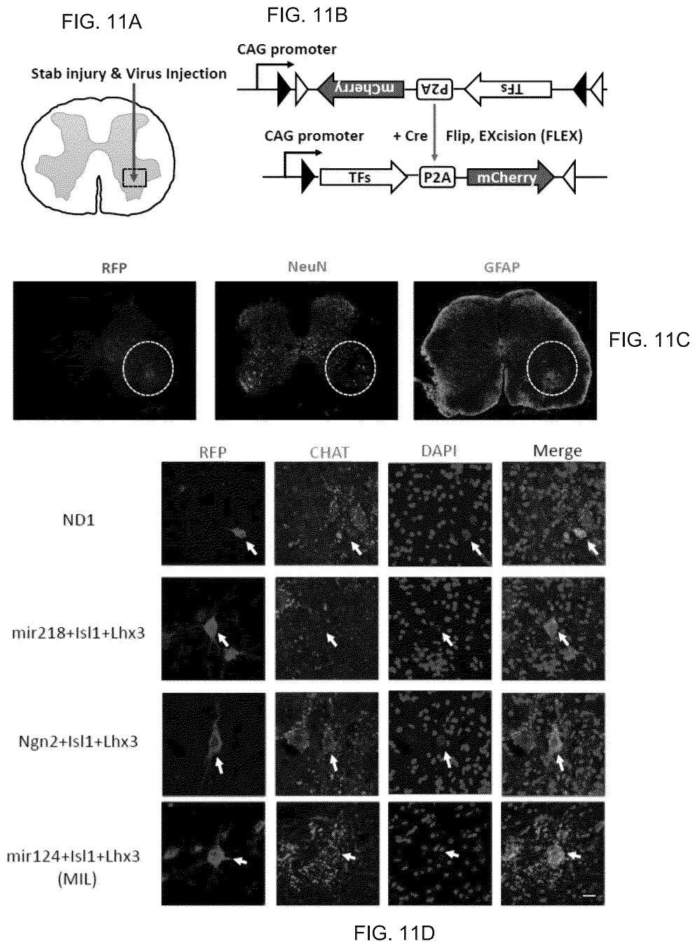

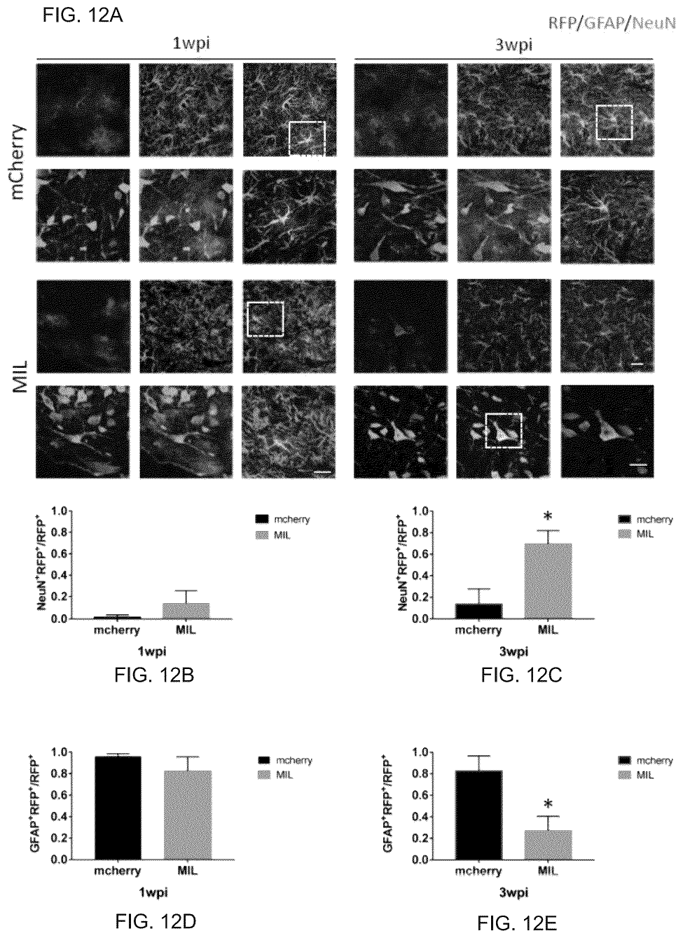

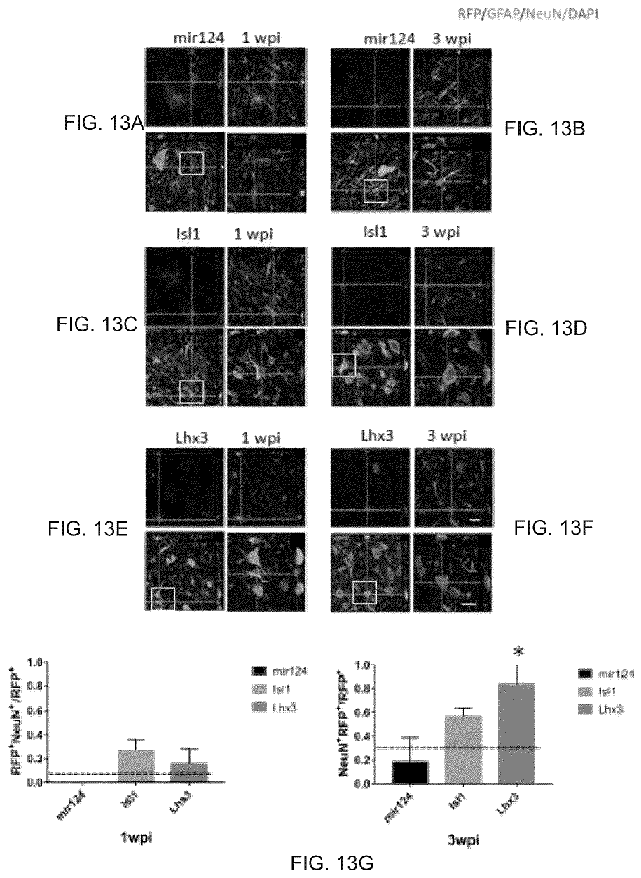

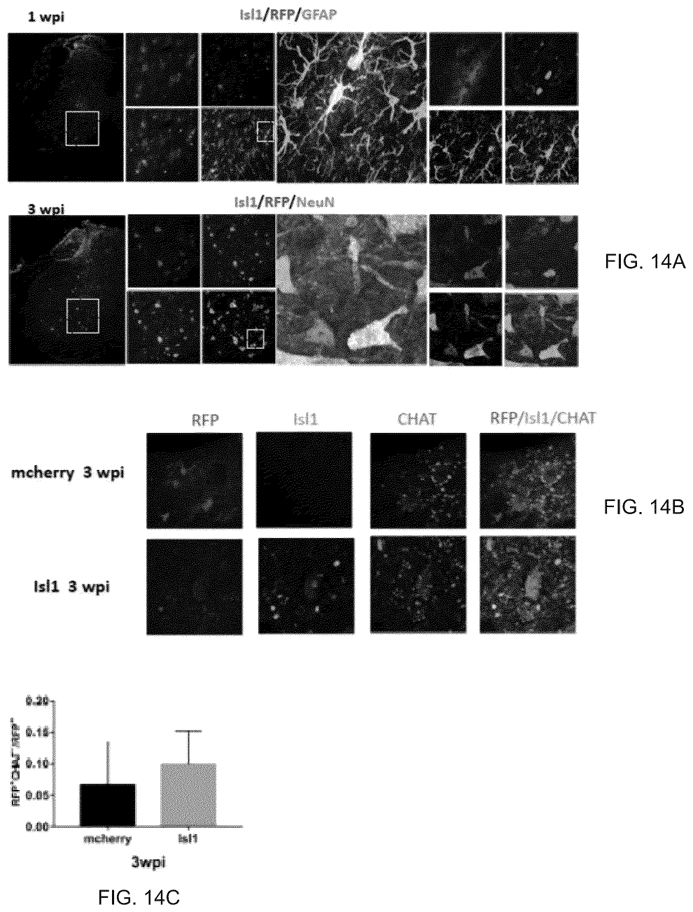

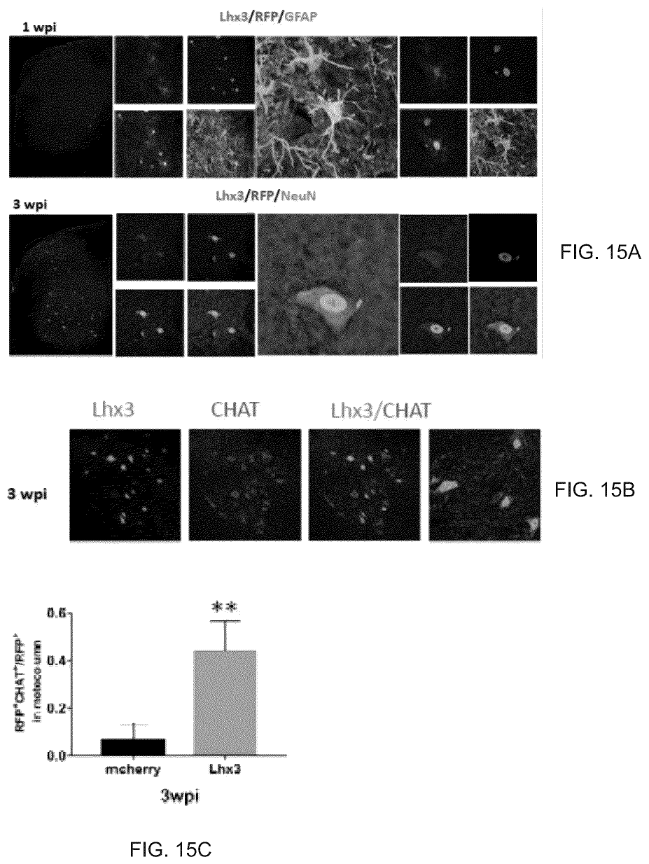

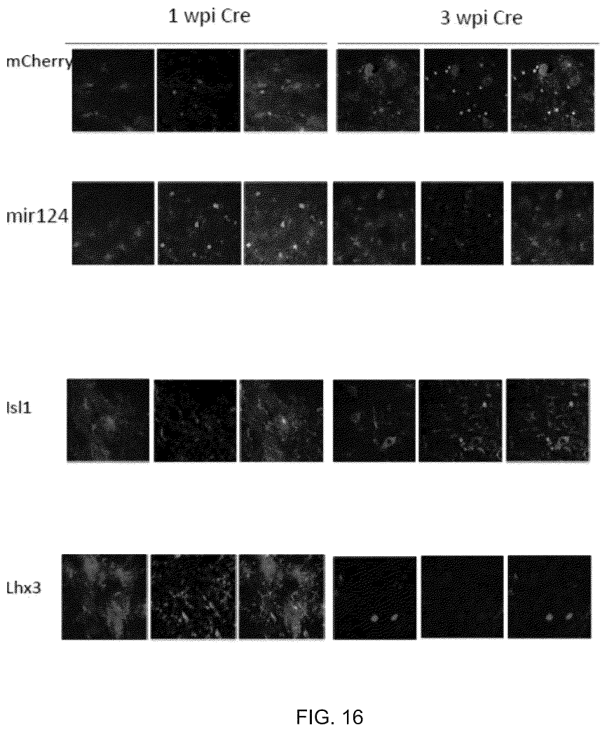

Nucleic acid driving expression of Mir124, NeuroD1, Isl1, Lhx3, Ngn2, or their combinations were introduced into the mouse ventral horn and found to convert astrocytes into neurons, with some of the converted neurons display motor neuron properties by immunostaining positive for ChAT, a typical motoneuron marker (FIGS. 11-16).

### Example 7—NeuroD1 and Dlx2-Mediated Cell Conversion

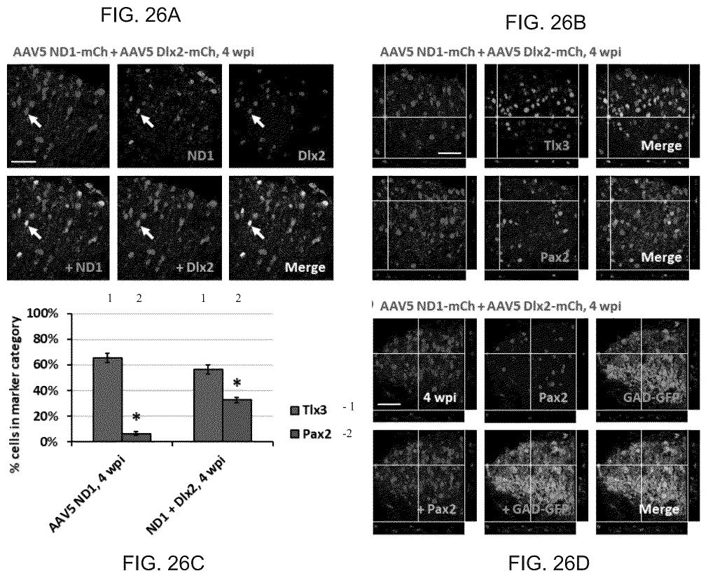

The following was performed to investigate whether it was possible to increase the proportion of GABAergic neurons by combining NeuroD1 with other transcription factors. A 1:1 ratio of AAV5 FLEX-NeuroD1-mCherry and AAV5 FLEX-Dlx2-mCherry in combination with AAV5-GFAP-Cre (FIG. 26; n=3) was used for injection. First, co-expression of NeuroD1 and Dlx2 was confirmed with immunostaining after viral infection (FIG. 26A). Immunostaining experiments demonstrated that many NeuroD1+Dlx2-converted neurons were Tlx3⁺ or Pax2⁺ neurons (FIG. 26B). Quantitative analysis revealed that 32.5±2.1% of NeuroD1+Dlx2-converted neurons were Pax2⁺ neurons (FIG. 26C), a 5-fold increase compared to that generated by NeuroD1 alone (6.3%; p=0.05, Kruskal-Wallis H-test). The percentage of Tlx3⁺ neurons generated by NeuroD1+Dlx2 was 56.2±3.4% (FIG. 26C). The GABAergic identity of the NeuroD1+Dlx2-converted neurons was further confirmed in GAD-GFP mice, where NeuroD1+Dlx2-converted neurons were both positive for both Pax2 and GAD-GFP+(FIG. 26D; n=3; 4 wpi). These results demonstrate that the ratio of newly converted Tlx3⁺ versus Pax2⁺ neurons in the dorsal horn of spinal cord can be determined through the combinations of NeuroD1 and Dlx2 transcription factors.

### Example 8—Treating ALS

The mouse model for ALS (SOD1*G93A) was used to investigate a gene therapy treatment using in vivo astrocyte-to-neuron conversion technology to regenerate motor neurons in the spinal cord and restore motor functions in ALS mice.

AAV9-GFAP-Cre+AAV9-Flex-mCherry were used to infect astrocytes as control experiments. AAV9-GFAP-Cre+AAV9-Flex-NeuroD1-mCherry, or AAV9-GFAP-Cre+AAV9-Flex-NeuroD1-GFP+AAV9-Flex-Isl1-mCherry, or AAV9-GFAP-Cre+AAV9-Flex-NeuroD1-GFP+AAV9-Flex-Lhx3-mCherry were employed to convert astrocytes into neurons in the spinal cord, through intra-spinal injection or intrathecal injection.

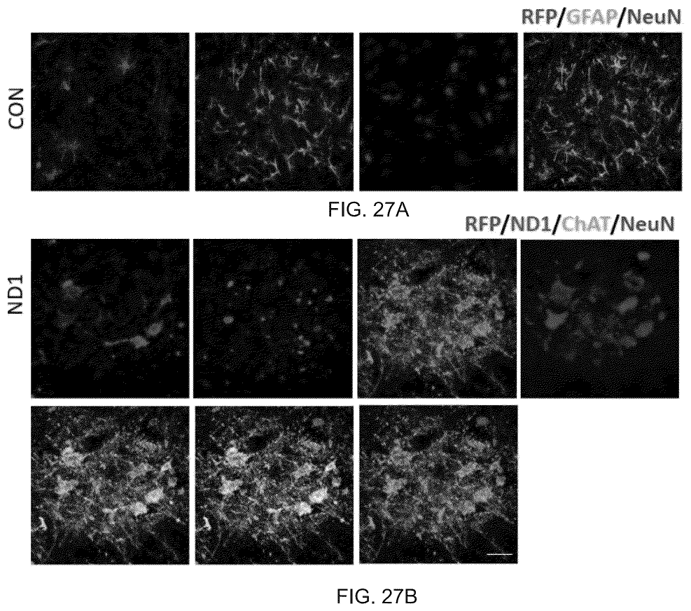

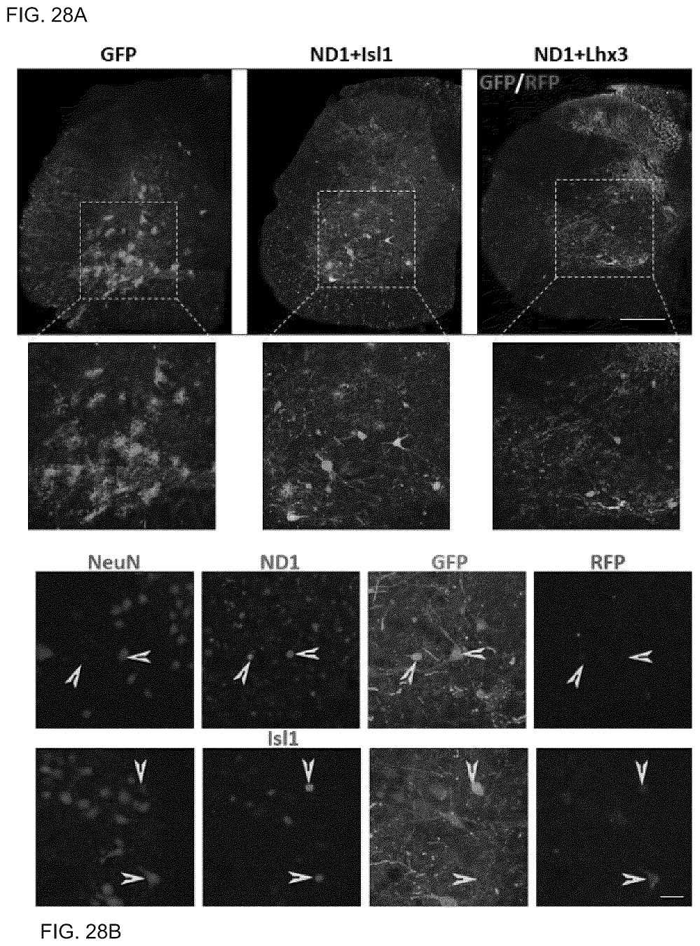

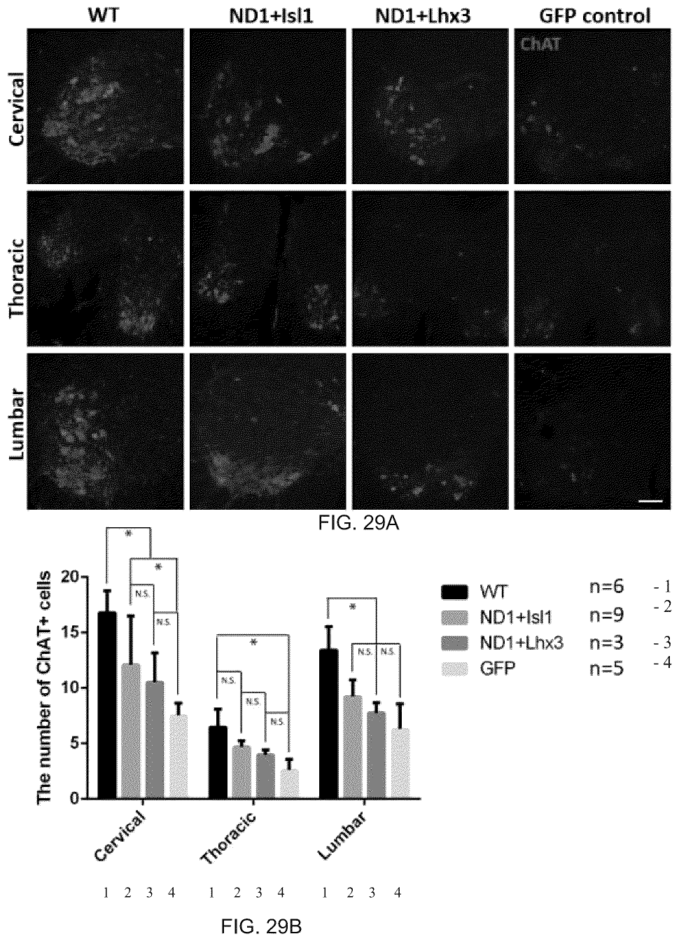

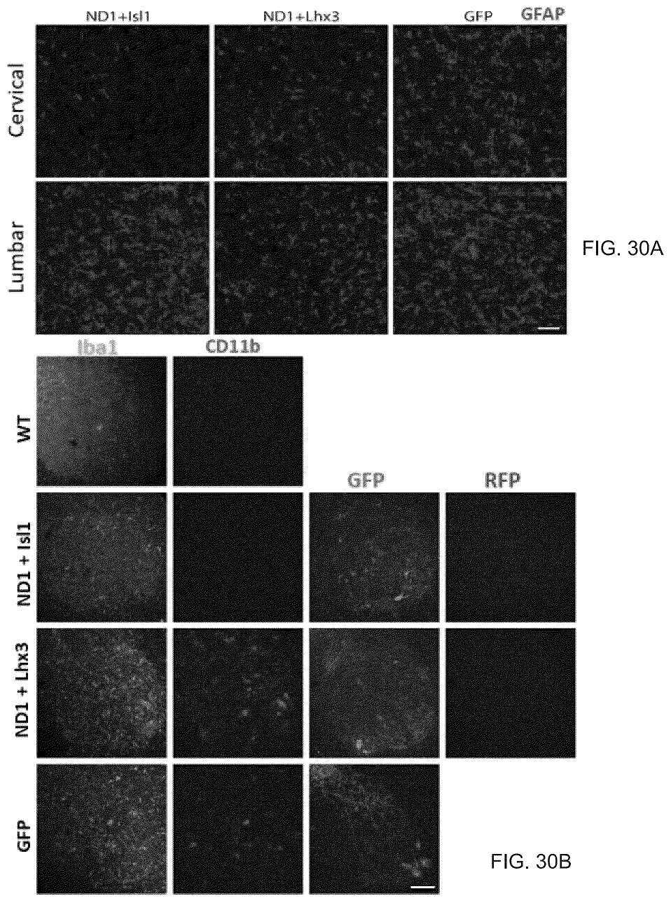

Injecting AAV9 expressing NeuroD1, or NeuroD1+Isl1, or NeuroD1+Lhx3, into the ventral horn of the spinal cord all generated ChAT+ motor neurons in ALS mice, but control mCherry alone AAV infected cells remained astrocytes (FIGS. 27-29).

Treatment with AAV9 expressing NeuroD1+Isl1 also reduced neuroinflammation shown as reduced Iba1 and CD11b signals (FIG. 30).

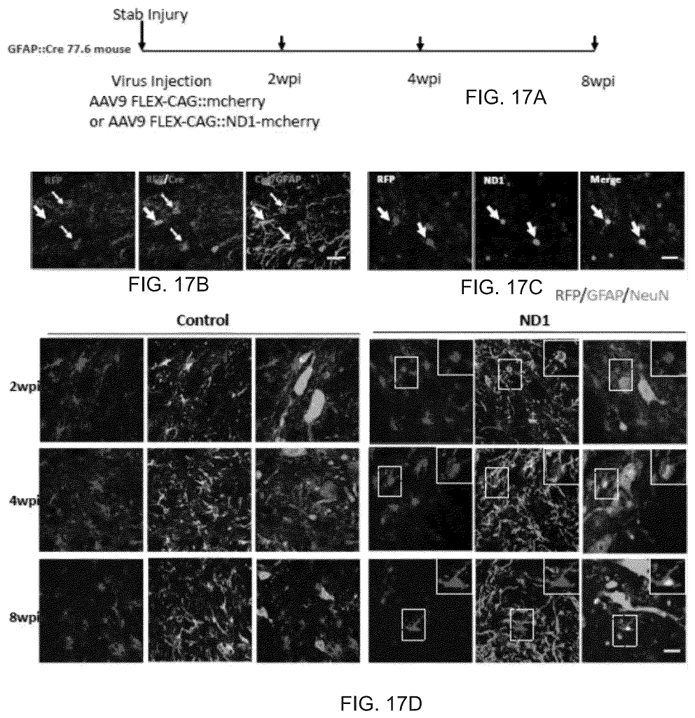

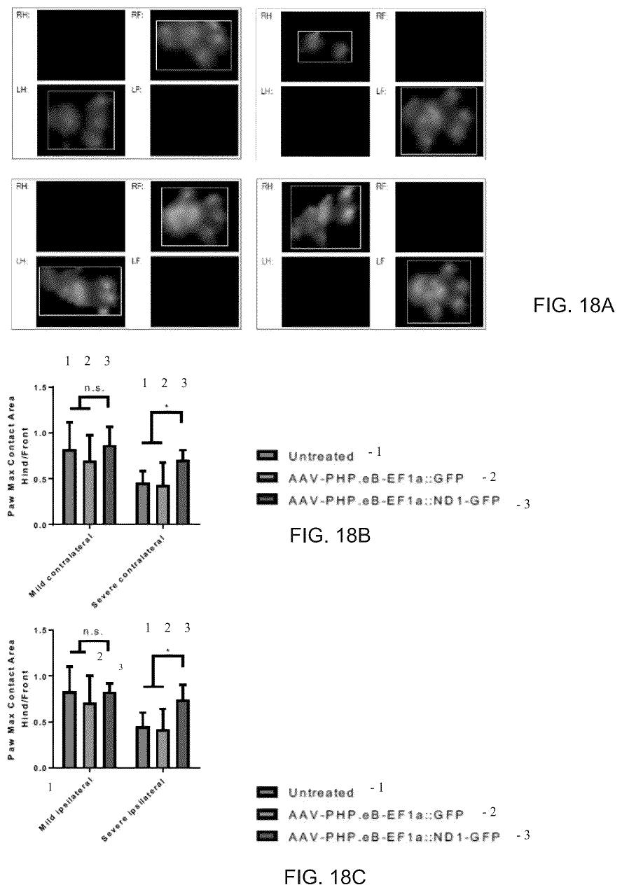

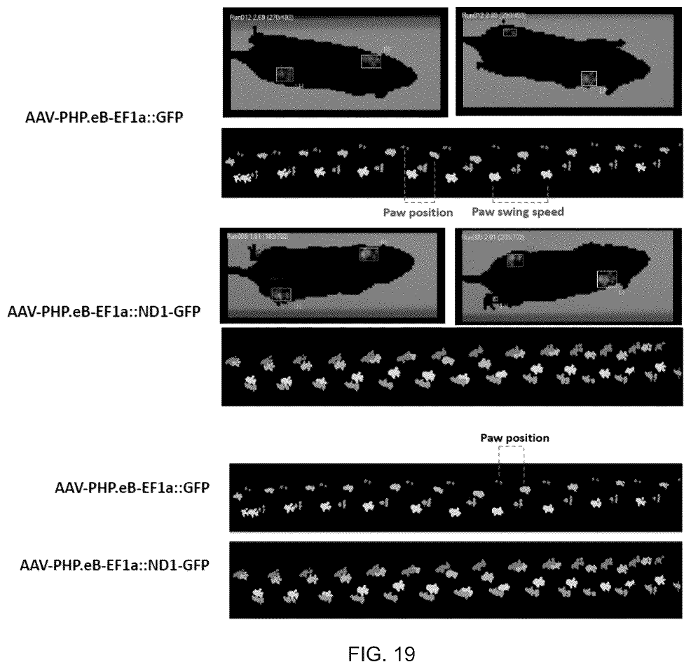

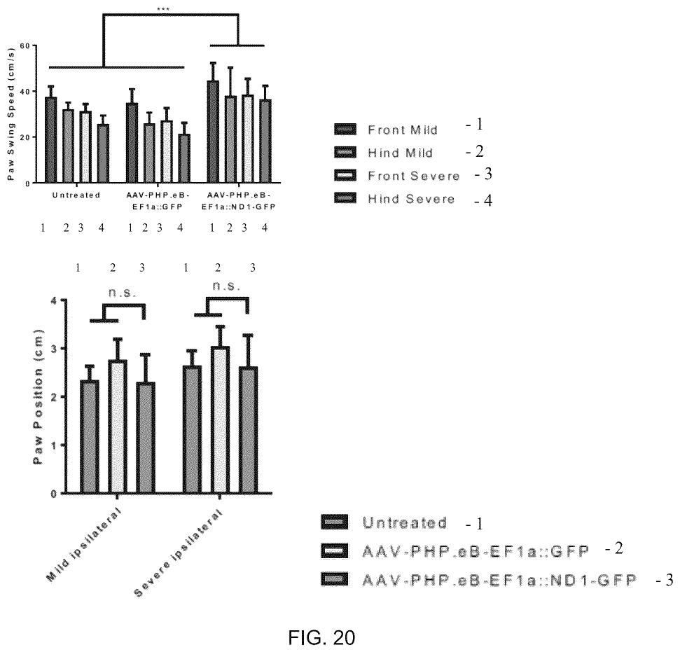

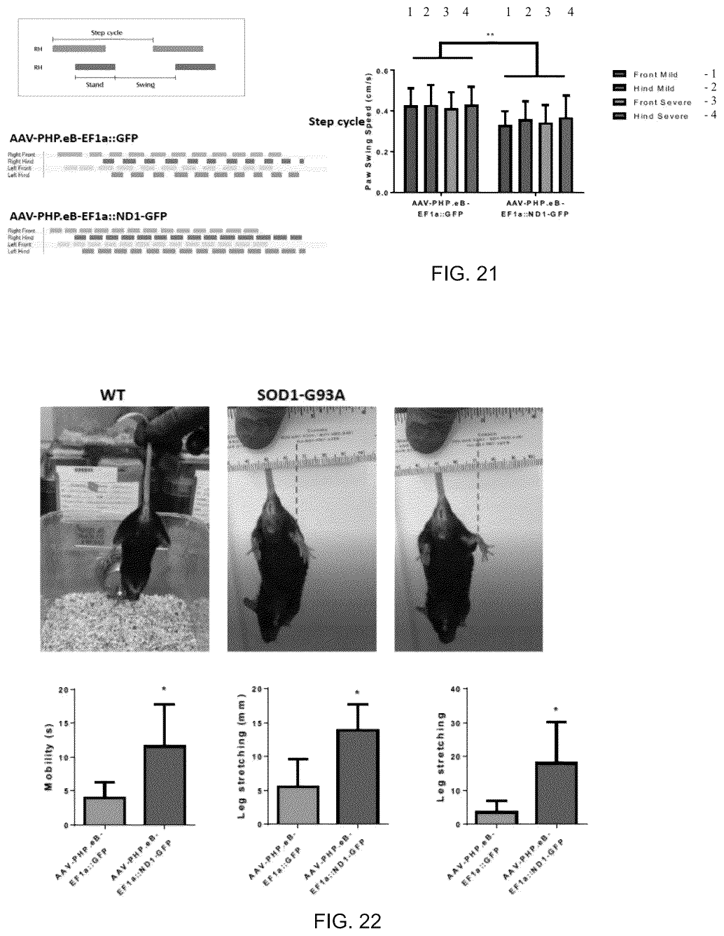

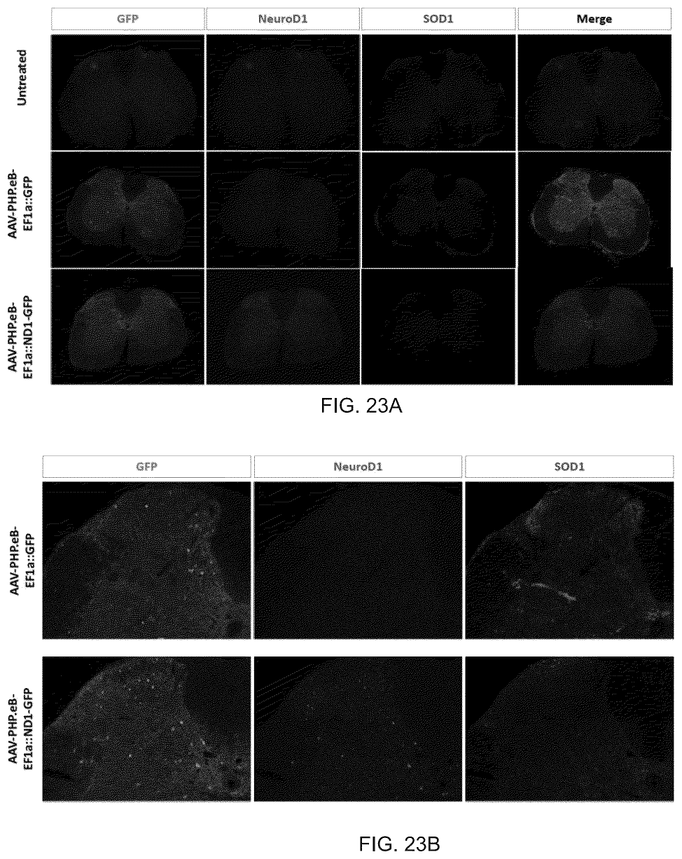

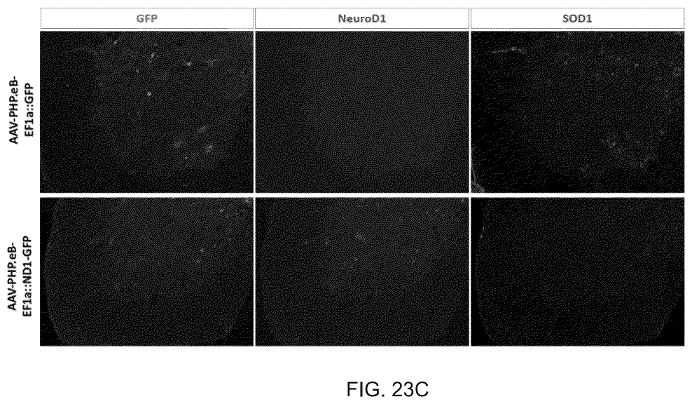

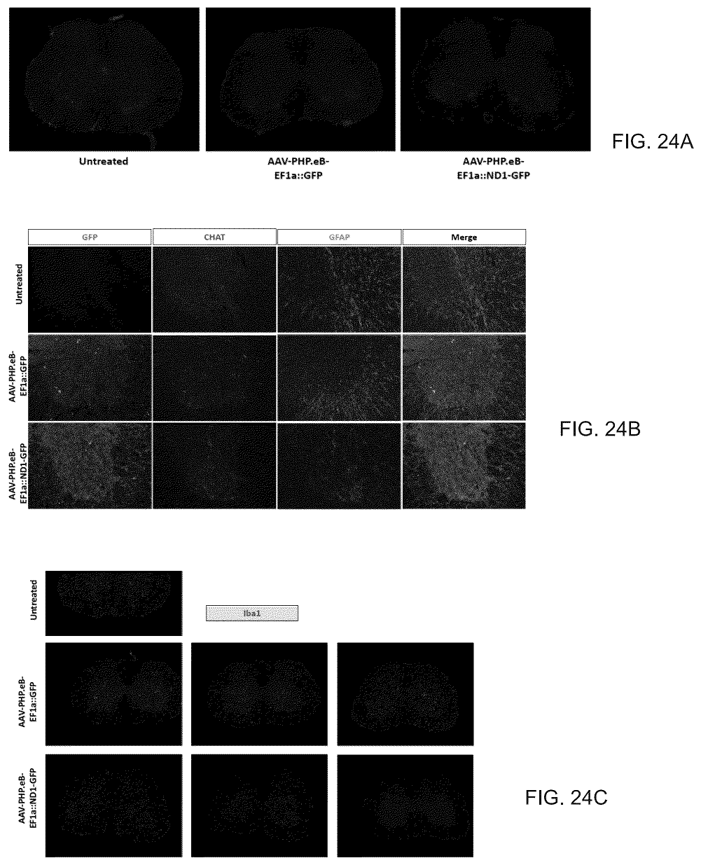

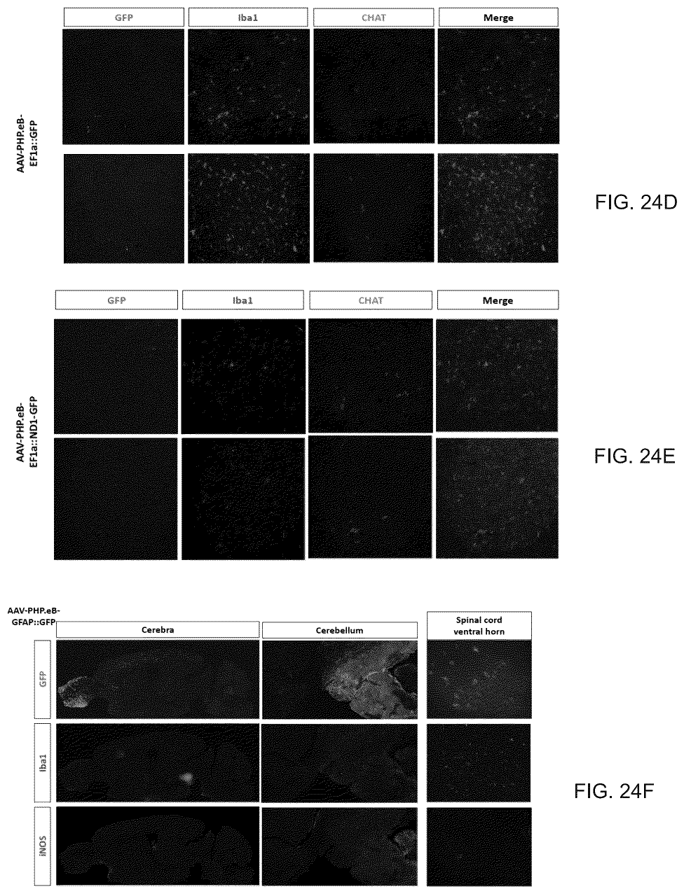

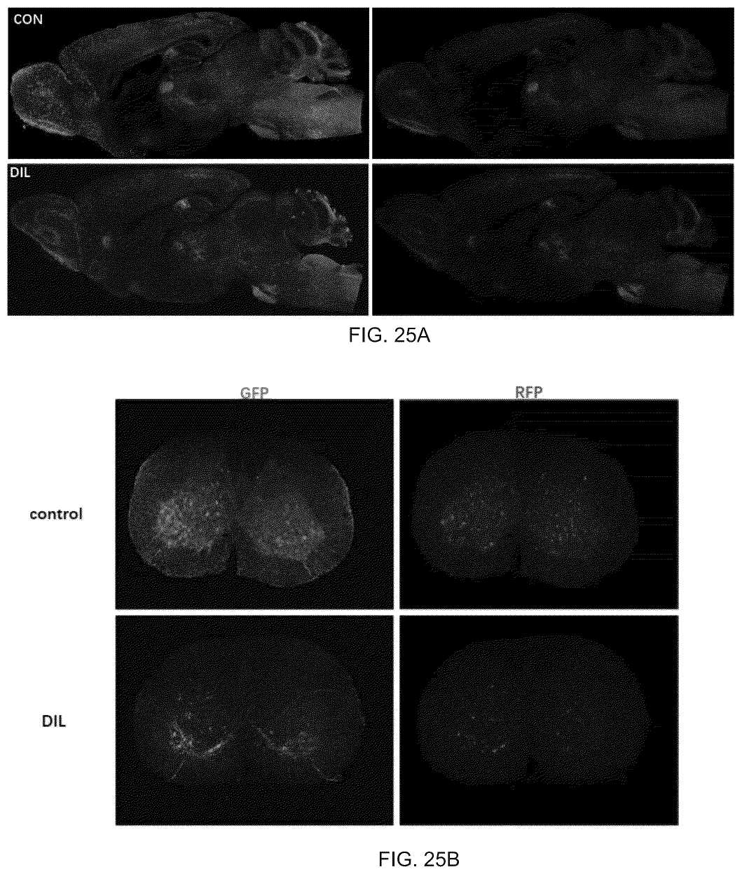

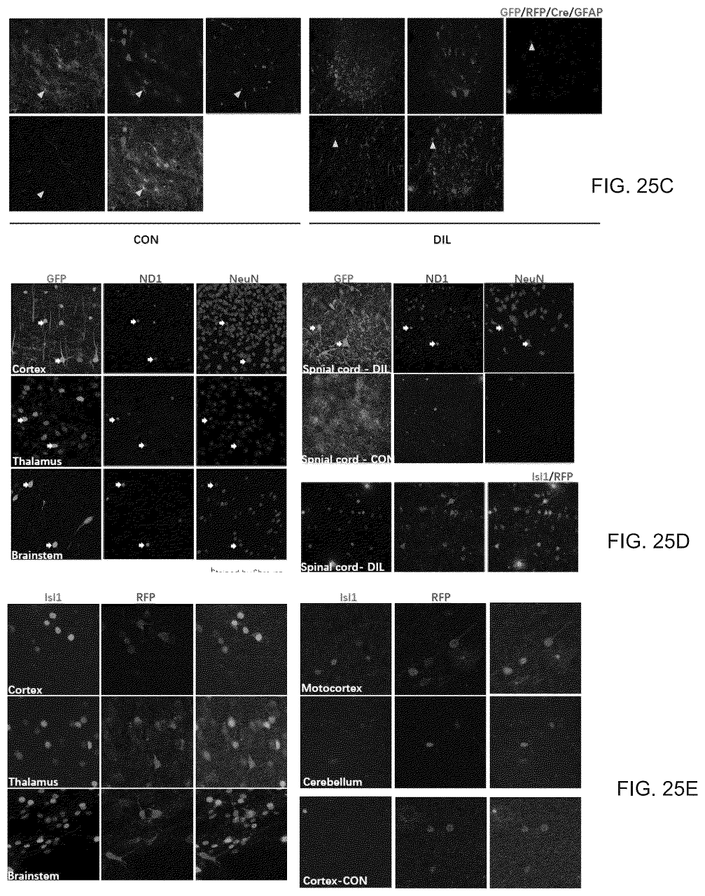

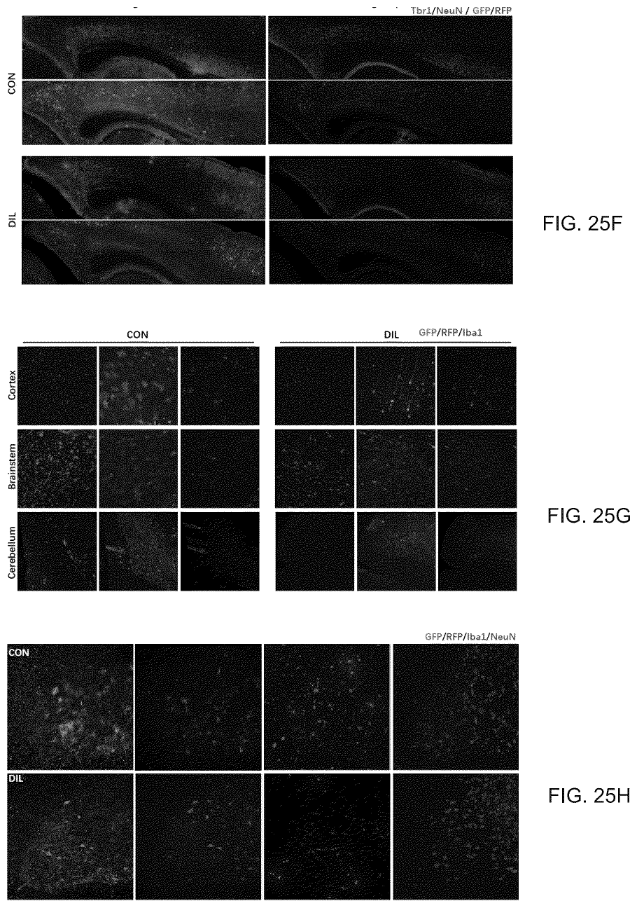

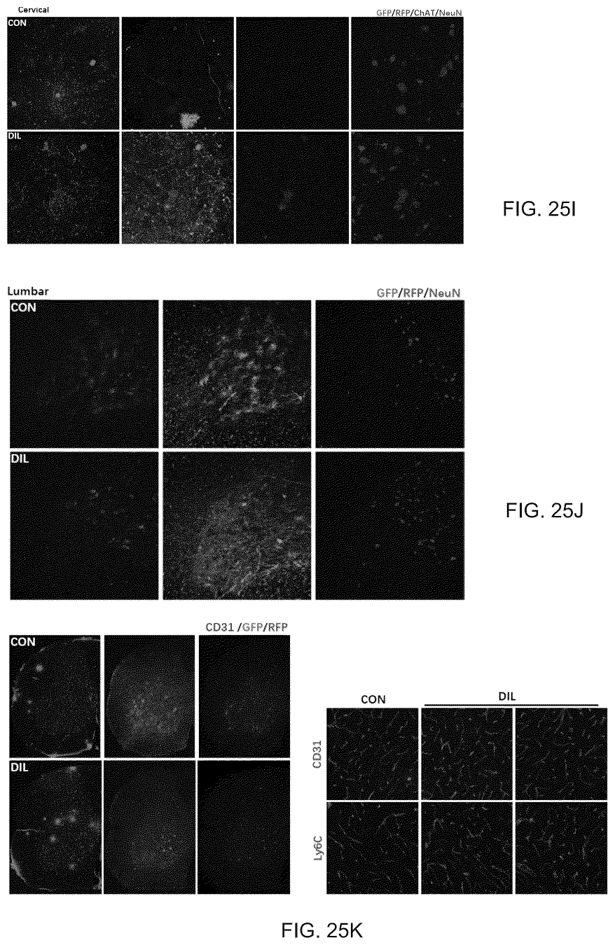

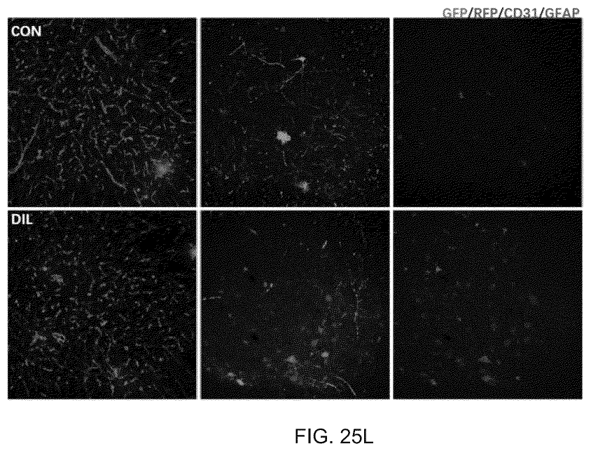

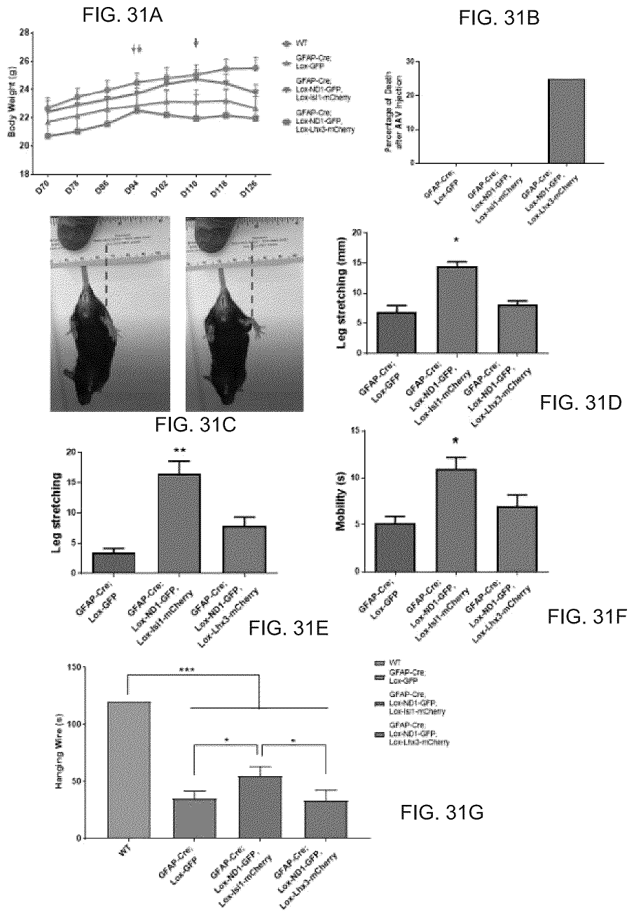

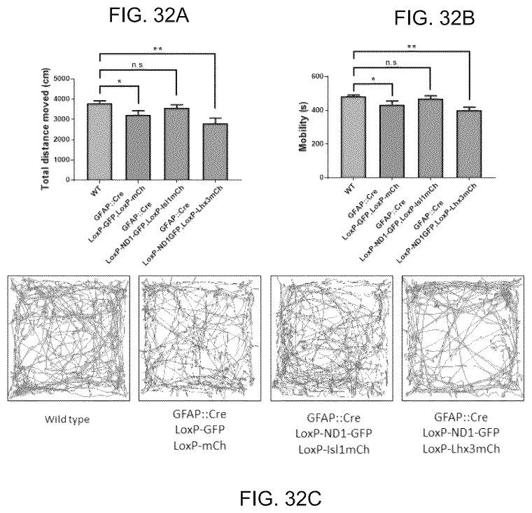

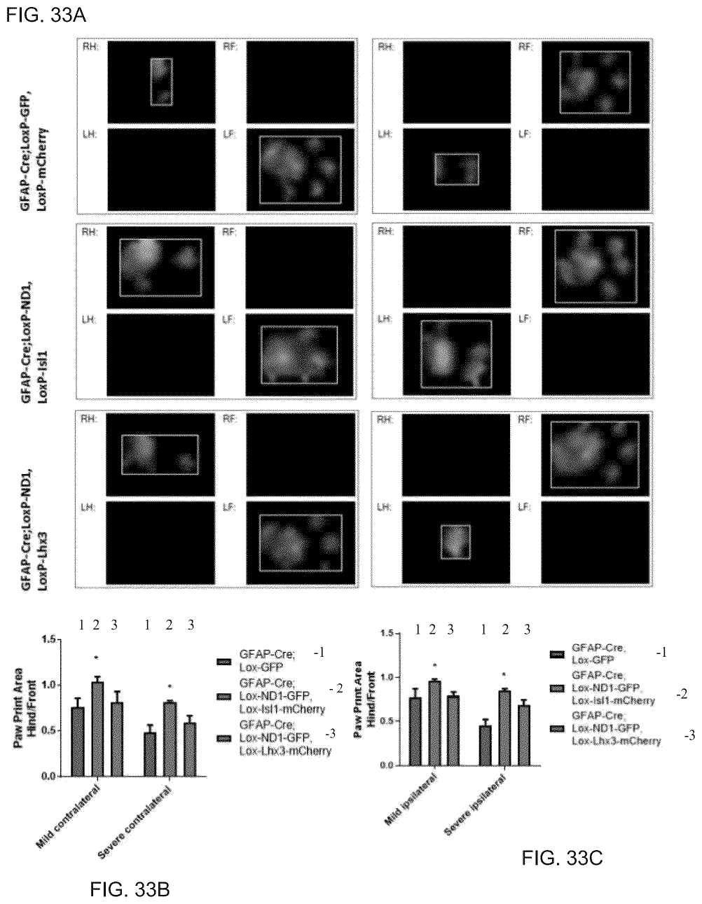

Moreover, treatment with AAV9 expressing NeuroD1+Isl1 also partially rescued the body weight, leg stretching ability, hanging wire duration, and open field mobility in ALS mice. Catwalk tests further found that treatment with AAV9 expressing NeuroD1+Isl1 increased paw print areas of the hind legs in ALS mice (FIG. 31-33). See, also, FIGS. 17-25. These results demonstrate that there were a little more motor neurons that appeared in cervical spinal cord. Motor neurons degenerated seriously in lumbar spinal cord in both control and NeuroD1-Isl1-Lhx3 (DIL) groups. The Iba1 signal revealed more microglia in the control group, and CD31 and Ly6C staining revealed that there was no huge difference in the blood vessels. The macrophage marker CD11b also was examined with combinations of factors that included NeuroD1+Isl1 and NeuroD1+Lhx3. Of these combinations, NeuroD1+Isl1 resulted in a greater reduction of CD11b signal, compared to the NeuroD1+Lhx3 group or GFP control group.

Taken together, these results confirm that gene therapy treatment can significantly convert astrocytes into neurons and that the regenerated motor neurons can improve motor functions in ALS mice. See, also, FIGS. 17-25.

### Animal Use

In this example, B6.Cg-Tg (SOD1*G93A) dl1Gur/J mice (The Jackson Laboratory) were mated with C57BL/6J females (The Jackson Laboratory) to obtain mice on a pure background. Mice were genotyped by PCR against human SOD1 after weaning (P21-27), and the littermates without mutation were used as normal mice. Mice were housed in a 12 hour light/dark cycle and supplied with sufficient food and water.

### Laminectomy, Injury, and Stereotaxic Viral Injection

SOD1G93A mice were anesthetized by intraperitoneal injection of ketamine/xylazine (80-120 mg/kg ketamine; 10-16 mg/kg xylazine), followed by fur trimming on the back, and placement into a stereotaxic setup. Artificial eye ointment was applied to cover the eye for protection purposes. A laminectomy was then performed at the Ti1-L1 vertebrae to expose the spinal cord. 1.0 μL of AAV9-GFAP-Cre+AAV9-Flex-NeuroD1-mCherry or AAV9-GFAP-Cre+AAV9-Flex-mCherry were injected into the ventral horn of spinal cord (0.45 mm lateral to the central artery with a depth of 0.9 mm), through a 50 μL Hamilton syringe with a 34-gauge injection needle at a rate of 0.05 μL/minute. After injection, the needle was kept in place for three minutes to prevent drawing out the virus and then slowly withdrawn. The surgical area was then treated with antibiotic ointment. The mice were kept on a heating pad and treated with Carprofen for pain relieve via subcutaneous injection (5 mg/kg) on the day of surgery and drinking water (10 mg/kg) for three days after surgery and closely monitored for one week to ensure full recovery of health.

### Intrathecal Injection

For intrathecal injections, 9-week-old mice were anesthetized using isoflurane. Ten microliters of AAV-PHP.eB-GFAP-Cre+AAV-PHP.eB-Flex-TFs-GFP/mCherry or +AAV-PHP.eB-Flex-GFP/mCherry were injected into the mouse lumbar subarachnoid space using a 25 μL Hamilton syringe with a 31-gauge injection needle. Briefly, mice were anesthetized with 3% isoflurane and shaved 2*2 cm² of fur at the posterior end of the animal near the tail to facilitate a batter visualization during needle insertion. Mice were placed in a nose cone for a continued isoflurane administration during the procedure in prone position, and the isoflurane was reduced to 1.5%. An artificial eye ointment was applied to cover the eye for protection purposes. The needle was carefully inserted between the groove of L5 and L6 vertebrae, and the mice were observe for a tail flick as a sign of a successful entry into the lumbar cistern. Once tail flick was observed, the syringe was stabilizes and the injection proceeded slowly. This injection was repeated twice every 24 hours to achieve optimal amount of virus (totally 2×10¹⁰ genome copies virus per mouse). All the injections were done with a timer to achieve a constant injection speed, inject 10 mL in 2 minutes. The syringe was removed after 1 minute to minimize CSF and vector leakage. Mice were maintained in the same position for 5 additional minutes. After surgery, animals were housed in cage with free access to food and water till end point.

### Body Weight

The body weight of each mouse was recorded every 8 days for 8 weeks from day 70 to day 126.

### Animal Behavior

### Open Field Test

Open field test was conducted in an opaque, open acrylic box (40×40×40 cm) in a brightly lit room. Mice were habituated to the testing environment for 1 hour in the testing room prior the test. Each individual mouse was randomly placed at one corner of the box in a dark room with red light. Video camera recorded horizontal movement of the mouse for 10 minutes. Total distance traveled and duration of movement were measured by Noldus EthoVision XT software. The open field apparatus was cleaned with 70% ethanol between each trail.

### Catwalk Test

Mice were habituated at the testing room for 1 hour before the test. The catwalk was conducted in the Noldus CatWalk XT system. Each individual mouse was placed at the entrance of the walkway with a straight line to guild the movement. The mouse traveled freely on the walkway. Mouse traveling with a constant speed was as a successful trail. Three trails were recorded with the illuminated footprints technology. Paw print area, swing speed, and step cycle were measured by the CatWalk XT software. The catwalk apparatus was cleaned with 70% ethanol between each trail.

### Wire Hanging Test

To test muscle strength, the wire hanging test was conducted. Each individual mouse was placed on a wired mesh. Until the mouse stopped moving, the mesh was gently turned around. The time of mouse falling onto a soft surface was measured.

### Leg Stretching Test

At the end point (22 weeks), each individual mouse was suspended by the tail from a lever for about 30 seconds. Video camera recorded the movement of the leg. The Leg stretching frequency, distance, and the duration of movement were measured.

### Example 9—Additional Embodiments

Embodiment 1. A method for treating a mammal having a spinal cord injury (SCI), wherein said method comprises administering a composition comprising exogenous nucleic acid encoding a Neuronal Differentiation 1 (NeuroD1) polypeptide or a biologically active fragment thereof to said mammal.
Embodiment 2. The method of embodiment 1, wherein said mammal is a human.
Embodiment 3. The method of embodiment 1, wherein the spinal cord injury is due to a condition selected from the group consisting of: ischemic stroke; hemorrhagic stroke; physical injury; concussion; contusion; blast; penetration; tumor; inflammation; infection; traumatic spinal injury; ischemic or hemorrhagic myelopathy (spinal cord infarction); global ischemia as caused by cardiac arrest or severe hypotension (shock); hypoxic-ischemic encephalopathy as caused by hypoxia, hypoglycemia, or anemia; CNS embolism as caused by infective endocarditis or atrial myxoma; fibrocartilaginous embolic myelopathy; CNS thrombosis as caused by pediatric leukemia; cerebral venous sinus thrombosis as caused by nephrotic syndrome (kidney disease), chronic inflammatory disease, pregnancy, use of estrogen-based contraceptives, meningitis, dehydration; or a combination of any two or more thereof.
Embodiment 4. The method of embodiment 1, wherein said administering step comprises delivering an expression vector comprising a nucleic acid encoding a NeuroD1 polypeptide or a biologically active fragment thereof to the spinal cord.
Embodiment 5. The method of embodiment 1, wherein said administering step comprises delivering a recombinant viral expression vector comprising a nucleic acid encoding a NeuroD1 polypeptide or a biologically active fragment thereof to the spinal cord.
Embodiment 6. The method of embodiment 1, wherein said administering step comprises delivering a recombinant adeno-associated virus expression vector comprising a nucleic acid encoding a NeuroD1 polypeptide or a biologically active fragment thereof to the spinal cord.
Embodiment 7. The method of embodiment 6, wherein the adeno-associated virus is an AAV.PHP.eB.
Embodiment 8. The method of any of embodiments 1-7, wherein said administering step comprises administering a recombinant expression vector comprising a nucleic acid sequence encoding NeuroD1 polypeptide, wherein the nucleic acid sequence encoding NeuroD1 polypeptide comprises a nucleic acid sequence selected from the group consisting of: a nucleic acid sequence encoding SEQ ID NO:2 or a functional fragment thereof; a nucleic acid sequence encoding SEQ ID NO:4 or a functional fragment thereof; SEQ ID NO:1 or a functional fragment thereof; SEQ ID NO:3 or a functional fragment thereof; and a nucleic acid sequence encoding a protein which has 70%, 71%, 72%, 73%, 74%, 75%, 76%, 77%, 78%, 79%, 81%, 82%, 83%, 84%, 85%, 86%, 87%, 88%, 89%, 90%, 91%, 92%, 93%, 94%, 95%, 96%, 97%, 98%, 99%, or greater, identity to SEQ ID NO: 2 or SEQ ID NO: 4, or a functional fragment thereof.
Embodiment 9. The method of any one of embodiments 1-8, wherein said administering step comprises a stereotactic injection to the spinal cord.
Embodiment 10. The method of any one of embodiments 1-8, wherein said administering step comprises an intravenous injection or intravenous infusion.
Embodiment 11. A method of treating a mammal having a spinal cord injury, wherein said method comprises administering a pharmaceutical composition comprising a pharmaceutically acceptable carrier containing adeno-associated virus particles comprising a nucleic acid encoding a NeuroD1 polypeptide or a biologically active fragment thereof to the spinal cord of said mammal.
Embodiment 12. The method of embodiment 11, wherein the pharmaceutical composition comprises about 1 μL to about 500 μL of a pharmaceutically acceptable carrier containing adeno-associated virus at a concentration of 10¹⁰-10¹⁴ adeno-associated virus particles/mL of carrier comprising a nucleic acid encoding a NeuroD1 polypeptide or a biologically active fragment thereof.
Embodiment 13. The method of embodiment 11 or 12, wherein the pharmaceutical composition is injected in the spinal cord of said mammal at a controlled flow rate of about 0.1 L/minute to about 5 μL/minute.
Embodiment 14. A method for treating a mammal having spinal cord injury, wherein said method comprises administering a composition comprising exogenous nucleic acid encoding mir124, exogenous nucleic acid encoding a ISL LIM Homeobox 1 (Isl1) polypeptide or a biologically active fragment thereof, and exogenous nucleic acid encoding a LIM Homeobox 3 (Lhx3) polypeptide or biologically active fragment thereof to the spinal cord of said mammal.
Embodiment 15. The method of embodiment 14, wherein said mammal is a human.
Embodiment 16. The method of embodiment 14, wherein said administering step comprises delivering (i) an expression vector comprising a nucleic acid encoding mir124, (ii) an expression vector comprising a nucleic acid encoding a Isl1 polypeptide or a biologically active fragment thereof, and (iii) an expression vector comprising a nucleic acid encoding a polypeptide or biologically active fragment thereof Lhx3 to the spinal cord of said mammal.
Embodiment 17. The method of embodiment 14, wherein said administering step comprises delivering (i) a recombinant viral expression vector comprising a nucleic acid encoding mir124, (ii) a recombinant viral expression vector comprising a nucleic acid encoding a Isl1 polypeptide or biologically active fragment thereof, and (iii) a recombinant viral expression vector comprising a nucleic acid encoding a Lhx3 polypeptide or a biologically active fragment thereof to the spinal cord of said mammal.
Embodiment 18. The method of embodiment 14, wherein said administering step comprises delivering (i) a recombinant adeno-associated virus expression vector comprising a nucleic acid encoding mir124, (ii) a recombinant adeno-associated virus expression vector comprising a nucleic acid encoding a Isl1 polypeptide or a biologically active fragment thereof, and (iii) a recombinant adeno-associated virus expression vector comprising a nucleic acid encoding a Lhx3 polypeptide or biologically active fragment thereof to the spinal cord of said mammal.
Embodiment 19. The method of embodiment 14, wherein said administering step comprises delivering an expression vector comprising a nucleic acid encoding mir124, a Isl1 polypeptide or a biologically active fragment thereof, and a Lhx3 polypeptide or a biologically active fragment thereof to the spinal cord of said mammal.
Embodiment 20. The method of embodiment 14, wherein said administering step comprises delivering a recombinant viral expression vector comprising a nucleic acid encoding mir124, a Isl1 polypeptide or a biologically active fragment thereof, and a Lhx3 polypeptide or a biologically active fragment thereof to the spinal cord of said mammal.
Embodiment 21. The method of embodiment 14, wherein said administering step comprises delivering a recombinant adeno-associated virus expression vector comprising a nucleic acid encoding mir124, a Isl1 polypeptide or biologically active fragment thereof, and a Lhx3 polypeptide or a biologically active fragment thereof to the spinal cord of said mammal.
Embodiment 22. The method of embodiment 14, wherein said administering step further comprises administering therapeutically effective doses of one or more of exogenous nucleic acid encoding a Neurogenin 2 (Ngn2) polypeptide or a biologically active fragment thereof, mir218, and a nucleic acid encoding a NeuroD1 polypeptide or a biologically active fragment thereof in combination with any combination of mir124, a Isl1 polypeptide or a biologically active fragment thereof, or a Lhx3 polypeptide or a biologically active fragment thereof to the spinal cord of said mammal.
Embodiment 23. The method of embodiments 18 or 21, wherein the adeno-associated virus is an AAV.PHP.eB.
Embodiment 24. A method for treating a mammal having Amyotrophic lateral sclerosis (ALS), wherein said method comprises administering a composition comprising exogenous nucleic acid encoding a NeuroD1 polypeptide or a biologically active fragment thereof to the central nervous system of said mammal.
Embodiment 25. The method of embodiment 24, wherein said mammal is a human.
Embodiment 26. The method of embodiment 24, wherein said administering step comprises delivering an expression vector comprising a nucleic acid encoding a NeuroD1 polypeptide or a biologically active fragment thereof to the brain.
Embodiment 27. The method of embodiment 24, wherein said administering step comprises delivering a recombinant viral expression vector comprising a nucleic acid encoding a NeuroD1 polypeptide or a biologically active fragment thereof to the brain.
Embodiment 28. The method of embodiment 24, wherein said administering step comprises delivering a recombinant adeno-associated virus expression vector comprising a nucleic acid encoding a NeuroD1 polypeptide or a biologically active fragment thereof to the brain.
Embodiment 29. The method of embodiment 28, wherein the adeno-associated virus is an AAV.PHP.eB.
Embodiment 30. The method of any one of embodiments 24-29, wherein said administering step comprises administering a recombinant expression vector comprising a nucleic acid sequence encoding NeuroD1 protein, wherein the nucleic acid sequence encoding NeuroD1 protein comprises a nucleic acid sequence selected from the group consisting of: a nucleic acid sequence encoding SEQ ID NO:2 or a functional fragment thereof; a nucleic acid sequence encoding SEQ ID NO:4 or a functional fragment thereof; SEQ ID NO:1 or a functional fragment thereof; SEQ ID NO:3 or a functional fragment thereof; and a nucleic acid sequence encoding a protein which has 70%, 71%, 72%, 73%, 74%, 75%, 76%, 77%, 78%, 79%, 81%, 82%, 83%, 84%, 85%, 86%, 87%, 88%, 89%, 90%, 91%, 92%, 93%, 94%, 95%, 96%, 97%, 98%, 99%, or greater, identity to SEQ ID NO: 2 or SEQ ID NO: 4, or a functional fragment thereof.
Embodiment 31. The method of any one of embodiments 24-30, wherein said administering step comprises a stereotactic intracranial injection.
Embodiment 32. The method of embodiment 31, wherein said administering step comprises two or more stereotactic intracranial injections.
Embodiment 33. The method of any one of embodiments 24-30, wherein said administering step comprises a retro-orbital injection.
Embodiment 34. A method of treating a mammal having ALS, wherein said method comprises administering a pharmaceutical composition comprising a pharmaceutically acceptable carrier containing adeno-associated virus particles comprising a nucleic acid encoding NeuroD1 to the central nervous system of said mammal.
Embodiment 35. The method of embodiment 34, wherein the pharmaceutical composition comprises about 1 μL to about 500 μL of a pharmaceutically acceptable carrier containing adeno-associated virus at a concentration of 10¹⁰-10¹⁴ adeno-associated virus particles/ml of carrier comprising a nucleic acid encoding a NeuroD1 polypeptide.
Embodiment 36. The method of embodiment 34 or 35, wherein the pharmaceutical composition is injected in the central nervous system of said mammal at a controlled flow rate of about 0.1 μL/minute to about 5 μL/minute.
Embodiment 37. A method for treating a mammal having Amyotrophic lateral sclerosis (ALS), wherein said method comprises administering a composition comprising exogenous nucleic acid encoding a NeuroD1 polypeptide or a biologically active fragment thereof, exogenous nucleic acid encoding an Isl1 polypeptide or a biologically active fragment thereof, and exogenous nucleic acid encoding a Lhx3 polypeptide or a biologically active fragment thereof to the central nervous system of said mammal.
Embodiment 38. The method of embodiment 37, wherein said mammal is a human.
Embodiment 39. The method of embodiment 37, wherein said administering step comprises delivering (i) an expression vector comprising a nucleic acid encoding a NeuroD1 polypeptide or a biologically active fragment thereof, (ii) an expression vector comprising a nucleic acid encoding a Isl1 polypeptide or a biologically active fragment thereof, and (iii) an expression vector comprising a nucleic acid encoding a Lhx3 polypeptide or a biologically active fragment thereof to the central nervous system of said mammal.
Embodiment 40. The method of embodiment 37, wherein said administering step comprises delivering (i) a recombinant viral expression vector comprising a nucleic acid encoding a NeuroD1 polypeptide or a biologically active fragment thereof, (ii) a recombinant viral expression vector comprising a nucleic acid encoding a Isl1 polypeptide or a biologically active fragment thereof, and (iii) a recombinant viral expression vector comprising a nucleic acid encoding a Lhx3 polypeptide or a biologically active fragment thereof to the central nervous system of said mammal.
Embodiment 41. The method of embodiment 37, wherein said administering step comprises delivering (i) a recombinant adeno-associated virus expression vector comprising a nucleic acid encoding a NeuroD1 polypeptide or a biologically active fragment thereof, (ii) a recombinant adeno-associated virus expression vector comprising a nucleic acid encoding a Isl1 polypeptide or a biologically active fragment thereof, and (iii) a recombinant adeno-associated virus expression vector comprising a nucleic acid encoding a Lhx3 polypeptide or a biologically active fragment thereof to the central nervous system of said mammal.
Embodiment 42. The method of embodiment 37, wherein said administering step comprises delivering an expression vector comprising a nucleic acid encoding a NeuroD1 polypeptide or a biologically active fragment thereof, a Isl1 polypeptide or a biologically active fragment thereof and a Lhx3 polypeptide or a biologically active fragment thereof to the central nervous system of said mammal.
Embodiment 43. The method of embodiment 37, wherein said administering step comprises delivering a recombinant viral expression vector comprising a nucleic acid encoding a NeuroD1 polypeptide or a biologically active fragment thereof, a Isl1 polypeptide or a biologically active fragment thereof, and a Lhx3 polypeptide or a biologically active fragment thereof to the central nervous system of said mammal.
Embodiment 44. The method of embodiment 37, wherein said administering step comprises delivering a recombinant adeno-associated virus expression vector comprising a nucleic acid encoding a NeuroD1 polypeptide or a biologically active fragment thereof, a Isl1 polypeptide or a biologically active fragment thereof, and a Lhx3 polypeptide or a biologically active fragment thereof to the central nervous system of said mammal.
Embodiment 45. The method of embodiment 37, wherein said administering step further comprises administering therapeutically effective doses of one or more of exogenous nucleic acid encoding Ngn2, mir218, and mir124 in combination with any combination of a NeuroD1 polypeptide or a biologically active fragment thereof, a Isl1 polypeptide or a biologically active fragment thereof, and a Lhx3 polypeptide or a biologically active fragment thereof to the central nervous system of said mammal.
Embodiment 46. The method of embodiment 41 or embodiment 44, wherein the adeno-associated virus is an AAV.PHP.eB.
Embodiment 47. A method for (1) regenerating dorsal spinal cord neurons, (2) generating new glutamatergic neurons, or (3) increasing circulation in the spinal cord within a mammal having a SCI and in need of said (1), (2), or (3), wherein said method comprises administering a composition comprising exogenous nucleic acid encoding a NeuroD1 polypeptide or a biologically active fragment thereof to said mammal, wherein (a) said spinal cord neurons are regenerated, (b) new glutamatergic neurons are generated, or (c) spinal cord circulation is increased.
Embodiment 48. The method of embodiment 47, wherein said mammal is a human.
Embodiment 49. The method of embodiment 47, wherein said administering step comprises delivering an expression vector comprising a nucleic acid encoding a NeuroD1 polypeptide or a biologically active fragment thereof to the spinal cord.
Embodiment 50. The method of embodiment 47, wherein said administering step comprises delivering a recombinant viral expression vector comprising a nucleic acid encoding a NeuroD1 polypeptide or a biologically active fragment thereof to the spinal cord.
Embodiment 51. The method of embodiment 47, wherein said administering step comprises delivering a recombinant adeno-associated virus expression vector comprising a nucleic acid encoding a NeuroD1 polypeptide or a biologically active fragment thereof to the spinal cord.
Embodiment 52. The method of embodiment 51, wherein the adeno-associated virus is an AAV.PHP.eB.
Embodiment 53. The method of any one of embodiments 47-52, wherein said administering step comprises administering a recombinant expression vector comprising a nucleic acid sequence encoding NeuroD1 polypeptide, wherein the nucleic acid sequence encoding NeuroD1 polypeptide comprises a nucleic acid sequence selected from the group consisting of: a nucleic acid sequence encoding SEQ ID NO:2 or a functional fragment thereof; a nucleic acid sequence encoding SEQ ID NO:4 or a functional fragment thereof; SEQ ID NO:1 or a functional fragment thereof; SEQ ID NO:3 or a functional fragment thereof; and a nucleic acid sequence encoding a protein which has 70%, 71%, 72%, 73%, 74%, 75%, 76%, 77%, 78%, 79%, 81%, 82%, 83%, 84%, 85%, 86%, 87%, 88%, 89%, 90%, 91%, 92%, 93%, 94%, 95%, 96%, 97%, 98%, 99%, or greater, identity to SEQ ID NO: 2 or SEQ ID NO: 4, or a functional fragment thereof.
Embodiment 54. The method of any one of embodiments 47-53, wherein said administering step comprises a stereotactic injection to the spinal cord.
Embodiment 55. The method of any one of embodiments 47-54, wherein said administering step comprises an intravenous injection or intravenous infusion.
Embodiment 56. A method for (1) generating motor neurons, (2) reducing the number of microglia, or (3) reducing the number of reactive astrocytes within a mammal having ALS disease and in need of said (1), (2), or (3), wherein said method comprises administering a composition comprising exogenous nucleic acid encoding a NeuroD1 polypeptide or a biologically active fragment thereof to said mammal, wherein (a) said motor neurons are generated, (b) the number of microglia is reduced, or (c) the number of reactive astrocytes is reduced.
Embodiment 57. The method of embodiment 56, wherein said mammal is a human.
Embodiment 58. The method of embodiment 56, wherein said administering step comprises delivering an expression vector comprising a nucleic acid encoding a NeuroD1 polypeptide or a biologically active fragment thereof to the spinal cord.
Embodiment 59. The method of embodiment 56, wherein said administering step comprises delivering a recombinant viral expression vector comprising a nucleic acid encoding a NeuroD1 polypeptide or a biologically active fragment thereof to the spinal cord.
Embodiment 60. The method of embodiment 56, wherein said administering step comprises delivering a recombinant adeno-associated virus expression vector comprising a nucleic acid encoding a NeuroD1 polypeptide or a biologically active fragment thereof to the spinal cord.
Embodiment 61. The method of embodiment 60, wherein the adeno-associated virus is an AAV.PHP.eB.
Embodiment 62. The method of any one of embodiments 56-61, wherein said administering step comprises administering a recombinant expression vector comprising a nucleic acid sequence encoding NeuroD1 polypeptide, wherein the nucleic acid sequence encoding NeuroD1 polypeptide comprises a nucleic acid sequence selected from the group consisting of: a nucleic acid sequence encoding SEQ ID NO:2 or a functional fragment thereof; a nucleic acid sequence encoding SEQ ID NO:4 or a functional fragment thereof; SEQ ID NO:1 or a functional fragment thereof; SEQ ID NO:3 or a functional fragment thereof; and a nucleic acid sequence encoding a protein which has 70%, 71%, 72%, 73%, 74%, 75%, 76%, 77%, 78%, 79%, 81%, 82%, 83%, 84%, 85%, 86%, 87%, 88%, 89%, 90%, 91%, 92%, 93%, 94%, 95%, 96%, 97%, 98%, 99%, or greater, identity to SEQ ID NO: 2 or SEQ ID NO: 4, or a functional fragment thereof.
Embodiment 63. The method of any one of embodiments 56-62, wherein said administering step comprises a stereotactic injection to the spinal cord.
Embodiment 64. The method of any one of embodiments 56-63, wherein said administering step comprises an intravenous injection or intravenous infusion.
Embodiment 65. A method for (1) regenerating dorsal spinal cord neurons, (2) generating new glutamatergic neurons, or (3) increasing circulation in the spinal cord within a mammal having a SCI and in need of said (1), (2), or (3), wherein said method comprises administering a composition comprising exogenous nucleic acid encoding a NeuroD1 polypeptide or a biologically active fragment thereof, exogenous nucleic acid encoding a Isl1 polypeptide or a biologically active fragment thereof, or exogenous nucleic acid encoding a Lhx3 polypeptide or a biologically active fragment thereof to said mammal, wherein (a) said spinal cord neurons are regenerated, (b) new glutamatergic neurons are generated, or (c) spinal cord circulation is increased.
Embodiment 66. The method of embodiment 65, wherein said mammal is a human.
Embodiment 67. The method of embodiment 66, wherein said administering step comprises delivering (i) an expression vector comprising a nucleic acid encoding a NeuroD1 polypeptide or a biologically active fragment thereof, (ii) an expression vector comprising a nucleic acid encoding a Isl1 polypeptide or a biologically active fragment thereof, or (iii) an expression vector comprising a nucleic acid encoding a Lhx3 polypeptide or a biologically active fragment thereof to the central nervous system of said mammal.
Embodiment 68. The method of embodiment 66, wherein said administering step comprises delivering (i) a recombinant viral expression vector comprising a nucleic acid encoding a NeuroD1 polypeptide or a biologically active fragment thereof, (ii) a recombinant viral expression vector comprising a nucleic acid encoding a Isl1 polypeptide or a biologically active fragment thereof, or (iii) a recombinant viral expression vector comprising a nucleic acid encoding a Lhx3 polypeptide or a biologically active fragment thereof to the central nervous system of said mammal.
Embodiment 69. The method of embodiment 66, wherein said administering step comprises delivering (i) a recombinant adeno-associated virus expression vector comprising a nucleic acid encoding a NeuroD1 polypeptide or a biologically active fragment thereof, (ii) a recombinant adeno-associated virus expression vector comprising a nucleic acid encoding a Isl1 polypeptide or a biologically active fragment thereof, or (iii) a recombinant adeno-associated virus expression vector comprising a nucleic acid encoding a Lhx3 polypeptide or a biologically active fragment thereof to the central nervous system of said mammal.
Embodiment 70. The method of embodiment 69, wherein the adeno-associated virus is an AAV.PHP.eB.
Embodiment 71. The method of any one of embodiments 65-70, wherein said administering step comprises a stereotactic injection to the spinal cord.
Embodiment 72. The method of any one of embodiments 65-71, wherein said administering step comprises an intravenous injection or intravenous infusion.
Embodiment 73. A method for treating a mammal having spinal cord injury, wherein said method comprises administering a composition comprising (a) exogenous nucleic acid encoding a NeuroD1 polypeptide or a biologically active fragment thereof and (b) exogenous nucleic acid encoding a Distal-Less Homeobox 2 (Dlx2) polypeptide or biologically active fragment thereof to the spinal cord of said mammal.
Embodiment 74. The method of embodiment 73, wherein said mammal is a human.
Embodiment 75. The method of embodiment 73, wherein said administering step comprises delivering (i) an expression vector comprising a nucleic acid a NeuroD1 polypeptide and (ii) an expression vector comprising a nucleic acid encoding a Dlx2 polypeptide or a biologically active fragment thereof to the spinal cord of said mammal.
Embodiment 76. The method of embodiment 73, wherein said administering step comprises delivering (i) a recombinant viral expression vector comprising a nucleic acid encoding a NeuroD1 polypeptide and (ii) a recombinant viral expression vector comprising a nucleic acid encoding a Dlx2 polypeptide or biologically active fragment thereof to the spinal cord of said mammal.
Embodiment 77. The method of embodiment 73, wherein said administering step comprises delivering (i) a recombinant adeno-associated virus expression vector comprising a nucleic acid encoding a NeuroD1 polypeptide and (ii) a recombinant adeno-associated virus expression vector comprising a nucleic acid encoding a Dlx2 polypeptide or a biologically active fragment thereof to the spinal cord of said mammal.
Embodiment 78. The method of embodiment 73, wherein said administering step comprises delivering an expression vector comprising a nucleic acid encoding a NeuroD1 polypeptide or a biologically active fragment thereof and a Dlx2 polypeptide or a biologically active fragment thereof to the spinal cord of said mammal.
Embodiment 79. The method of embodiment 73, wherein said administering step comprises delivering a recombinant viral expression vector comprising a nucleic acid encoding a NeuroD1 polypeptide or a biologically active fragment thereof and a Dlx2 polypeptide or a biologically active fragment thereof to the spinal cord of said mammal.
Embodiment 80. The method of embodiment 73, wherein said administering step comprises delivering a recombinant adeno-associated virus expression vector comprising a nucleic acid encoding a NeuroD1 polypeptide or biologically active fragment thereof and a Dlx2 polypeptide or a biologically active fragment thereof to the spinal cord of said mammal.
Embodiment 81. The method of embodiments 77 or 80, wherein the adeno-associated virus is an AAV.PHP.eB.
Embodiment 82. The method of any one of embodiments 73-81, wherein said administering step comprises administering a recombinant expression vector comprising a nucleic acid sequence encoding NeuroD1 polypeptide, wherein the nucleic acid sequence encoding NeuroD1 polypeptide comprises a nucleic acid sequence selected from the group consisting of: a nucleic acid sequence encoding SEQ ID NO:2 or a functional fragment thereof; a nucleic acid sequence encoding SEQ ID NO:4 or a functional fragment thereof; SEQ ID NO:1 or a functional fragment thereof; SEQ ID NO:3 or a functional fragment thereof; and a nucleic acid sequence encoding a protein which has 70%, 71%, 72%, 73%, 74%, 75%, 76%, 77%, 78%, 79%, 81%, 82%, 83%, 84%, 85%, 86%, 87%, 88%, 89%, 90%, 91%, 92%, 93%, 94%, 95%, 96%, 97%, 98%, 99%, or greater, identity to SEQ ID NO: 2 or SEQ ID NO: 4, or a functional fragment thereof.
Embodiment 83. The method of any one of embodiments 73-82, wherein said administering step comprises administering a recombinant expression vector comprising a nucleic acid sequence encoding Dlx2 polypeptide, wherein the nucleic acid sequence encoding Dlx2 polypeptide comprises a nucleic acid sequence selected from the group consisting of: a nucleic acid sequence encoding SEQ ID NO: 11 or a functional fragment thereof; a nucleic acid sequence encoding SEQ ID NO:13 or a functional fragment thereof; SEQ ID NO:10 or a functional fragment thereof; SEQ ID NO:12 or a functional fragment thereof; and a nucleic acid sequence encoding a protein which has 70%, 71%, 72%, 73%, 74%, 75%, 76%, 77%, 78%, 79%, 81%, 82%, 83%, 84%, 85%, 86%, 87%, 88%, 89%, 90%, 91%, 92%, 93%, 94%, 95%, 96%, 97%, 98%, 99%, or greater, identity to SEQ ID NO: 11 or SEQ ID NO: 13, or a functional fragment thereof.
Embodiment 84. A method for (1) regenerating dorsal spinal cord neurons, (2) generating new neurons, or (3) increasing circulation in the spinal cord within a mammal having a SCI and in need of said (1), (2), or (3), wherein said method comprises administering a composition comprising (i) exogenous nucleic acid encoding a NeuroD1 polypeptide or a biologically active fragment thereof and (ii) exogenous nucleic acid encoding a Dlx2 polypeptide or a biologically active fragment thereof, wherein (a) said spinal cord neurons are regenerated, (b) new neurons are generated, or (c) spinal cord circulation is increased.
Embodiment 85. The method of embodiment 84, wherein said mammal is a human.
Embodiment 86. The method of embodiment 84, wherein said administering step comprises delivering (i) an expression vector comprising a nucleic acid encoding a NeuroD1 polypeptide or a biologically active fragment thereof and (ii) an expression vector comprising a nucleic acid encoding a Dlx2 polypeptide or a biologically active fragment thereof to the central nervous system of said mammal.
Embodiment 87. The method of embodiment 84, wherein said administering step comprises delivering (i) a recombinant viral expression vector comprising a nucleic acid encoding a NeuroD1 polypeptide or a biologically active fragment thereof and (ii) a recombinant viral expression vector comprising a nucleic acid encoding a Dlx2 polypeptide or a biologically active fragment thereof to the central nervous system of said mammal.
Embodiment 88. The method of embodiment 84, wherein said administering step comprises delivering (i) a recombinant adeno-associated virus expression vector comprising a nucleic acid encoding a NeuroD1 polypeptide or a biologically active fragment thereof and (ii) a recombinant adeno-associated virus expression vector comprising a nucleic acid encoding a Dlx2 polypeptide or a biologically active fragment thereof to the central nervous system of said mammal.
Embodiment 89. The method of embodiment 84, wherein said administering step comprises delivering an expression vector comprising a nucleic acid encoding a NeuroD1 polypeptide or a biologically active fragment thereof and a Dlx2 polypeptide or a biologically active fragment thereof to the spinal cord of said mammal.
Embodiment 90. The method of embodiment 84, wherein said administering step comprises delivering a recombinant viral expression vector comprising a nucleic acid encoding a NeuroD1 polypeptide or a biologically active fragment thereof and a Dlx2 polypeptide or a biologically active fragment thereof to the spinal cord of said mammal.
Embodiment 91. The method of embodiment 84, wherein said administering step comprises delivering a recombinant adeno-associated virus expression vector comprising a nucleic acid encoding a NeuroD1 polypeptide or biologically active fragment thereof and a Dlx2 polypeptide or a biologically active fragment thereof to the spinal cord of said mammal.
Embodiment 92. The method of embodiments 88 or 91, wherein the adeno-associated virus is an AAV.PHP.eB.
Embodiment 93. The method of any one of embodiments 73-92, wherein said administering step comprises a stereotactic injection to the spinal cord.
Embodiment 94. The method of any one of embodiments 73-93, wherein said administering step comprises an intravenous injection or intravenous infusion.
Embodiment 95. The method of embodiment 84, wherein said new neurons are selected from the group consisting of glutamatergic neurons and GABAergic neurons.
Embodiment 96. The method of embodiment 95, wherein said new neurons are glutamatergic neurons.
Embodiment 97. The method of embodiment 95, wherein said new neurons are GABAergic neurons.
Embodiment 98. The method of embodiments 77 or 80, wherein the adeno-associated virus is an AAV serotype 5.
Embodiment 99. The method of embodiments 88 or 91, wherein the adeno-associated virus is an AAV serotype 5.
Embodiment 100. A method for treating a mammal having ALS, wherein said method comprises administering a composition comprising (a) exogenous nucleic acid encoding a NeuroD1 polypeptide or a biologically active fragment thereof and (b) exogenous nucleic acid encoding an Isl1 polypeptide or biologically active fragment thereof to said mammal.
Embodiment 101. The method of embodiment 100, wherein said mammal is a human.
Embodiment 102. The method of embodiment 100, wherein said administering step comprises delivering (i) an expression vector comprising a nucleic acid a NeuroD1 polypeptide and (ii) an expression vector comprising a nucleic acid encoding an Isl1 polypeptide or a biologically active fragment thereof to said mammal.
Embodiment 103. The method of embodiment 100, wherein said administering step comprises delivering (i) a recombinant viral expression vector comprising a nucleic acid encoding a NeuroD1 polypeptide and (ii) a recombinant viral expression vector comprising a nucleic acid encoding an Isl1 polypeptide or biologically active fragment thereof to said mammal.
Embodiment 104. The method of embodiment 100, wherein said administering step comprises delivering (i) a recombinant adeno-associated virus expression vector comprising a nucleic acid encoding a NeuroD1 polypeptide and (ii) a recombinant adeno-associated virus expression vector comprising a nucleic acid encoding an Isl1 polypeptide or a biologically active fragment thereof to the spinal cord of said mammal.
Embodiment 105. The method of embodiment 100, wherein said administering step comprises delivering an expression vector comprising a nucleic acid encoding a NeuroD1 polypeptide or a biologically active fragment thereof and an Isl1 polypeptide or a biologically active fragment thereof to said mammal.
Embodiment 106. The method of embodiment 100, wherein said administering step comprises delivering a recombinant viral expression vector comprising a nucleic acid encoding a NeuroD1 polypeptide or a biologically active fragment thereof and an Isl1 polypeptide or a biologically active fragment thereof to said mammal.
Embodiment 107. The method of embodiment 100, wherein said administering step comprises delivering a recombinant adeno-associated virus expression vector comprising a nucleic acid encoding a NeuroD1 polypeptide or biologically active fragment thereof and an Isl1 polypeptide or a biologically active fragment thereof to said mammal.
Embodiment 108. The method of embodiments 104 or 107, wherein said adeno-associated virus is an AAV.PHP.eB.
Embodiment 109. The method of any one of embodiments 100-108, wherein said administering step comprises administering a recombinant expression vector comprising a nucleic acid sequence encoding NeuroD1 polypeptide, wherein the nucleic acid sequence encoding NeuroD1 polypeptide comprises a nucleic acid sequence selected from the group consisting of: a nucleic acid sequence encoding SEQ ID NO:2 or a functional fragment thereof; a nucleic acid sequence encoding SEQ ID NO:4 or a functional fragment thereof; SEQ ID NO:1 or a functional fragment thereof; SEQ ID NO:3 or a functional fragment thereof; and a nucleic acid sequence encoding a protein which has 70%, 71%, 72%, 73%, 74%, 75%, 76%, 77%, 78%, 79%, 81%, 82%, 83%, 84%, 85%, 86%, 87%, 88%, 89%, 90%, 91%, 92%, 93%, 94%, 95%, 96%, 97%, 98%, 99%, or greater, identity to SEQ ID NO: 2 or SEQ ID NO: 4, or a functional fragment thereof.
Embodiment 110. The method of any one of embodiments 100-109, wherein said administering step comprises administering a recombinant expression vector comprising a nucleic acid sequence encoding an Isl1 polypeptide, wherein the nucleic acid sequence encoding an Isl1 polypeptide comprises a nucleic acid sequence selected from the group consisting of: a nucleic acid sequence encoding SEQ ID NO: 15 or a functional fragment thereof; a nucleic acid sequence encoding SEQ ID NO:17 or a functional fragment thereof; SEQ ID NO:14 or a functional fragment thereof; SEQ ID NO:16 or a functional fragment thereof; and a nucleic acid sequence encoding a protein which has 70%, 71%, 72%, 73%, 74%, 75%, 76%, 77%, 78%, 79%, 81%, 82%, 83%, 84%, 85%, 86%, 87%, 88%, 89%, 90%, 91%, 92%, 93%, 94%, 95%, 96%, 97%, 98%, 99%, or greater, identity to SEQ ID NO: 15 or SEQ ID NO: 17, or a functional fragment thereof.
Embodiment 111. A method for treating a mammal having spinal cord injury, wherein said method comprises administering a composition comprising (a) exogenous nucleic acid encoding a NeuroD1 polypeptide or a biologically active fragment thereof and (b) exogenous nucleic acid encoding an Isl1 polypeptide or biologically active fragment thereof to the spinal cord of said mammal.
Embodiment 112. The method of embodiment 111, wherein said mammal is a human.
Embodiment 113. The method of embodiment 111, wherein said administering step comprises delivering (i) an expression vector comprising a nucleic acid a NeuroD1 polypeptide and (ii) an expression vector comprising a nucleic acid encoding an Isl1 polypeptide or a biologically active fragment thereof to the spinal cord of said mammal.
Embodiment 114. The method of embodiment 111, wherein said administering step comprises delivering (i) a recombinant viral expression vector comprising a nucleic acid encoding a NeuroD1 polypeptide and (ii) a recombinant viral expression vector comprising a nucleic acid encoding an Isl1 polypeptide or biologically active fragment thereof to the spinal cord of said mammal.
Embodiment 115. The method of embodiment 111, wherein said administering step comprises delivering (i) a recombinant adeno-associated virus expression vector comprising a nucleic acid encoding a NeuroD1 polypeptide and (ii) a recombinant adeno-associated virus expression vector comprising a nucleic acid encoding an Isl1 polypeptide or a biologically active fragment thereof to the spinal cord of said mammal.
Embodiment 116. The method of embodiment 111, wherein said administering step comprises delivering an expression vector comprising a nucleic acid encoding a NeuroD1 polypeptide or a biologically active fragment thereof and an Isl1 polypeptide or a biologically active fragment thereof to the spinal cord of said mammal.
Embodiment 117. The method of embodiment 111, wherein said administering step comprises delivering a recombinant viral expression vector comprising a nucleic acid encoding a NeuroD1 polypeptide or a biologically active fragment thereof and an Isl1 polypeptide or a biologically active fragment thereof to the spinal cord of said mammal.
Embodiment 118. The method of embodiment 111, wherein said administering step comprises delivering a recombinant adeno-associated virus expression vector comprising a nucleic acid encoding a NeuroD1 polypeptide or biologically active fragment thereof and an Isl1 polypeptide or a biologically active fragment thereof to the spinal cord of said mammal.
Embodiment 119. The method of embodiments 115 or 118, wherein the adeno-associated virus is an AAV.PHP.eB.
Embodiment 120. The method of any one of embodiments 111-119, wherein said administering step comprises administering a recombinant expression vector comprising a nucleic acid sequence encoding NeuroD1 polypeptide, wherein the nucleic acid sequence encoding NeuroD1 polypeptide comprises a nucleic acid sequence selected from the group consisting of: a nucleic acid sequence encoding SEQ ID NO:2 or a functional fragment thereof; a nucleic acid sequence encoding SEQ ID NO:4 or a functional fragment thereof; SEQ ID NO:1 or a functional fragment thereof; SEQ ID NO:3 or a functional fragment thereof; and a nucleic acid sequence encoding a protein which has 70%, 71%, 72%, 73%, 74%, 75%, 76%, 77%, 78%, 79%, 81%, 82%, 83%, 84%, 85%, 86%, 87%, 88%, 89%, 90%, 91%, 92%, 93%, 94%, 95%, 96%, 97%, 98%, 99%, or greater, identity to SEQ ID NO: 2 or SEQ ID NO: 4, or a functional fragment thereof.
Embodiment 121. The method of any one of embodiments 111-120, wherein said administering step comprises administering a recombinant expression vector comprising a nucleic acid sequence encoding an Isl1 polypeptide, wherein the nucleic acid sequence encoding an Isl1 polypeptide comprises a nucleic acid sequence selected from the group consisting of: a nucleic acid sequence encoding SEQ ID NO: 15 or a functional fragment thereof; a nucleic acid sequence encoding SEQ ID NO:17 or a functional fragment thereof; SEQ ID NO:14 or a functional fragment thereof; SEQ ID NO:16 or a functional fragment thereof; and a nucleic acid sequence encoding a protein which has 70%, 71%, 72%, 73%, 74%, 75%, 76%, 77%, 78%, 79%, 81%, 82%, 83%, 84%, 85%, 86%, 87%, 88%, 89%, 90%, 91%, 92%, 93%, 94%, 95%, 96%, 97%, 98%, 99%, or greater, identity to SEQ ID NO: 15 or SEQ ID NO: 17, or a functional fragment thereof.

SEQUENCES
Human NeuroD1 nucleic acid sequence encoding human NeuroD1
protein - 1071 nucleotides, including stop codon
SEQ ID NO: 1
ATGACCAAATCGTACAGCGAGAGTGGGCTGATGGGCGAGCCTCAGCCCCAAGGTCCTCCAAGCT
GGACAGACGAGTGTCTCAGTTCTCAGGACGAGGAGCACGAGGCAGACAAGAAGGAGGACGACCT
CGAAGCCATGAACGCAGAGGAGGACTCACTGAGGAACGGGGGAGAGGAGGAGGACGAAGATGAG
GACCTGGAAGAGGAGGAAGAAGAGGAAGAGGAGGATGACGATCAAAAGCCCAAGAGACGCGGCC
CCAAAAAGAAGAAGATGACTAAGGCTCGCCTGGAGCGTTTTAAATTGAGACGCATGAAGGCTAA
CGCCCGGGAGCGGAACCGCATGCACGGACTGAACGCGGCGCTAGACAACCTGCGCAAGGTGGTG
CCTTGCTATTCTAAGACGCAGAAGCTGTCCAAAATCGAGACTCTGCGCTTGGCCAAGAACTACA
TCTGGGCTCTGTCGGAGATCCTGCGCTCAGGCAAAAGCCCAGACCTGGTCTCCTTCGTTCAGAC
GCTTTGCAAGGGCTTATCCCAACCCACCACCAACCTGGTTGCGGGCTGCCTGCAACTCAATCCT
CGGACTTTTCTGCCTGAGCAGAACCAGGACATGCCCCCCCACCTGCCGACGGCCAGCGCTTCCT
TCCCTGTACACCCCTACTCCTACCAGTCGCCTGGGCTGCCCAGTCCGCCTTACGGTACCATGGA
CAGCTCCCATGTCTTCCACGTTAAGCCTCCGCCGCACGCCTACAGCGCAGCGCTGGAGCCCTTC
TTTGAAAGCCCTCTGACTGATTGCACCAGCCCTTCCTTTGATGGACCCCTCAGCCCGCCGCTCA
GCATCAATGGCAACTTCTCTTTCAAACACGAACCGTCCGCCGAGTTTGAGAAAAATTATGCCTT
TACCATGCACTATCCTGCAGCGACACTGGCAGGGGCCCAAAGCCACGGATCAATCTTCTCAGGC
ACCGCTGCCCCTCGCTGCGAGATCCCCATAGACAATATTATGTCCTTCGATAGCCATTCACATC
ATGAGCGAGTCATGAGTGCCCAGCTCAATGCCATATTTCATGATTAG
Human NeuroD1 amino acid sequence - 356 amino acids - encoded
by SEQ ID NO: 1
SEQ ID NO: 2
MTKSYSESGLMGEPQPQGPPSWTDECLSSQDEEHEADKKEDDLEAMNAEEDSLRNGGEEEDEDE
DLEEEEEEEEEDDDQKPKRRGPKKKKMTKARLERFKLRRMKANARERNRMHGLNAALDNLRKVV
PCYSKTQKLSKIETLRLAKNYIWALSEILRSGKSPDLVSFVQTLCKGLSQPTTNLVAGCLQLNP
RTFLPEQNQDMPPHLPTASASFPVHPYSYQSPGLPSPPYGTMDSSHVFHVKPPPHAYSAALEPF
FESPLTDCTSPSFDGPLSPPLSINGNFSFKHEPSAEFEKNYAFTMHYPAATLAGAQSHGSIFSG
TAAPRCEIPIDNIMSFDSHSHHERVMSAQLNAIFHD
Mouse NeuroD1 nucleic acid sequence encoding mouse NeuroD1
protein - 1074 nucleotides, including stop codon
SEQ ID NO: 3
ATGACCAAATCATACAGCGAGAGCGGGCTGATGGGCGAGCCTCAGCCCCAAGGTCCCCCAAGCT
GGACAGATGAGTGTCTCAGTTCTCAGGACGAGGAACACGAGGCAGACAAGAAAGAGGACGAGCT
TGAAGCCATGAATGCAGAGGAGGACTCTCTGAGAAACGGGGGAGAGGAGGAGGAGGAAGATGAG
GATCTAGAGGAAGAGGAGGAAGAAGAAGAGGAGGAGGAGGATCAAAAGCCCAAGAGACGGGGTC
CCAAAAAGAAAAAGATGACCAAGGCGCGCCTAGAACGTTTTAAATTAAGGCGCATGAAGGCCAA
CGCCCGCGAGCGGAACCGCATGCACGGGCTGAACGCGGCGCTGGACAACCTGCGCAAGGTGGTA
CCTTGCTACTCCAAGACCCAGAAACTGTCTAAAATAGAGACACTGCGCTTGGCCAAGAACTACA
TCTGGGCTCTGTCAGAGATCCTGCGCTCAGGCAAAAGCCCTGATCTGGTCTCCTTCGTACAGAC
GCTCTGCAAAGGTTTGTCCCAGCCCACTACCAATTTGGTCGCCGGCTGCCTGCAGCTCAACCCT
CGGACTTTCTTGCCTGAGCAGAACCCGGACATGCCCCCGCATCTGCCAACCGCCAGCGCTTCCT
TCCCGGTGCATCCCTACTCCTACCAGTCCCCTGGACTGCCCAGCCCGCCCTACGGCACCATGGA
CAGCTCCCACGTCTTCCACGTCAAGCCGCCGCCACACGCCTACAGCGCAGCTCTGGAGCCCTTC
TTTGAAAGCCCCCTAACTGACTGCACCAGCCCTTCCTTTGACGGACCCCTCAGCCCGCCGCTCA
GCATCAATGGCAACTTCTCTTTCAAACACGAACCATCCGCCGAGTTTGAAAAAAATTATGCCTT
TACCATGCACTACCCTGCAGCGACGCTGGCAGGGCCCCAAAGCCACGGATCAATCTTCTCTTCC
GGTGCCGCTGCCCCTCGCTGCGAGATCCCCATAGACAACATTATGTCTTTCGATAGCCATTCGC
ATCATGAGCGAGTCATGAGTGCCCAGCTTAATGCCATCTTTCACGATTAG
Mouse NeuroD1 amino acid sequence - 357 amino acids - encoded by
SEQ ID NO: 3
SEQ ID NO: 4
MTKSYSESGLMGEPQPQGPPSWTDECLSSQDEEHEADKKEDELEAMNAEEDSLRNGGEEEEEDE
DLEEEEEEEEEEEDQKPKRRGPKKKKMTKARLERFKLRRMKANARERNRMHGLNAALDNLRKVV
PCYSKTQKLSKIETLRLAKNYIWALSEILRSGKSPDLVSFVQTLCKGLSQPTTNLVAGCLQLNP
RTFLPEQNPDMPPHLPTASASFPVHPYSYQSPGLPSPPYGTMDSSHVFHVKPPPHAYSAALEPF
FESPLTDCTSPSFDGPLSPPLSINGNFSFKHEPSAEFEKNYAFTMHYPAATLAGPQSHGSIFSS
GAAAPRCEIPIDNIMSFDSHSHHERVMSAQLNAIFHD
Mouse LCN2 promoter
SEQ ID NO: 5
GCAGTGTGGAGACACACCCACTTTCCCCAAGGGCTCCTGCTCCCCCAAGTGATCCCCTTATCCT
CCGTGCTAAGATGACACCGAGGTTGCAGTCCTTACCTTTGAAAGCAGCCACAAGGGCGTGGGGG
TGCACACCTTTAATCCCAGCACTCGGGAGGCAGAGGCAGGCAGATTTCTGAGTTCGAGACCAGC
CTGGTCTACAAAGTGAATTCCAGGACAGCCAGGGCTATACAGAGAAACCCTGTCTTGAAAAAAA
AAGAGAAAGAAAAAAGAAAAAAAAAAATGAAAGCAGCCACATCTAAGGACTACGTGGCACAGGA
GAGGGTGAGTCCCTGAGAGTTCAGCTGCTGCCCTGTCTGTTCCTGTAAATGGCAGTGGGGTCAT
GGGAAAGTGAAGGGGCTCAAGGTATTGGACACTTCCAGGATAATCTTTTGGACGCCTCACCCTG
TGCCAGGACCAAGGCTGAGCTTGGCAGGCTCAGAACAGGGTGTCCTGTTCTTCCCTGTCTAAAA
CATTCACTCTCAGCTTGCTCACCCTTCCCCAGACAAGGAAGCTGCACAGGGTCTGGTGTTCAGA
TGGCTTTGGCTTACAGCAGGTGTGGGTGTGGGGTAGGAGGCAGGGGGTAGGGGTGGGGGAAGCC
TGTACTATACTCACTATCCTGTTTCTGACCCTCTAGGACTCCTACAGGGTTATGGGAGTGGACA
GGCAGTCCAGATCTGAGCTGCTGACCCACAAGCAGTGCCCTGTGCCTGCCAGAATCCAAAGCCC
TGGGAATGTCCCTCTGGTCCCCCTCTGTCCCCTGCAGCCCTTCCTGTTGCTCAACCTTGCACAG
TTCCGACCTGGGGGAGAGAGGGACAGAAATCTTGCCAAGTATTTCAACAGAATGTACTGGCAAT
TACTTCATGGCTTCCTGGACTTGGTAAAGGATGGACTACCCCGCCCAACAGGGGGGCTGGCAGC
CAGGTAGGCCCATAAAAAGCCCGCTGGGGAGTCCTCCTCACTCTCTGCTCTTCCTCCTCCAGCA
CACATCAGACCTAGTAGCTGTGGAAACCA
Human GFAP promoter
SEQ ID NO: 6
GTCTGCAAGCAGACCTGGCAGCATTGGGCTGGCCGCCCCCCAGGGCCTCCTCTTCATGCCCAGT
GAATGACTCACCTTGGCACAGACACAATGTTCGGGGTGGGCACAGTGCCTGCTTCCCGCCGCAC
CCCAGCCCCCCTCAAATGCCTTCCGAGAAGCCCATTGAGTAGGGGGCTTGCATTGCACCCCAGC
CTGACAGCCTGGCATCTTGGGATAAAAGCAGCACAGCCCCCTAGGGGCTGCCCTTGCTGTGTGG
CGCCACCGGCGGTGGAGAACAAGGCTCTATTCAGCCTGTGCCCAGGAAAGGGGATCAGGGGATG
CCCAGGCATGGACAGTGGGTGGCAGGGGGGGAGAGGAGGGCTGTCTGCTTCCCAGAAGTCCAAG
GACACAAATGGGTGAGGGGACTGGGCAGGGTTCTGACCCTGTGGGACCAGAGTGGAGGGCGTAG
ATGGACCTGAAGTCTCCAGGGACAACAGGGCCCAGGTCTCAGGCTCCTAGTTGGGCCCAGTGGC
TCCAGCGTTTCCAAACCCATCCATCCCCAGAGGTTCTTCCCATCTCTCCAGGCTGATGTGTGGG
AACTCGAGGAAATAAATCTCCAGTGGGAGACGGAGGGGTGGCCAGGGAAACGGGGCGCTGCAGG
AATAAAGACGAGCCAGCACAGCCAGCTCATGCGTAACGGCTTTGTGGAGCTGTCAAGGCCTGGT
CTCTGGGAGAGAGGCACAGGGAGGCCAGACAAGGAAGGGGTGACCTGGAGGGACAGATCCAGGG
GCTAAAGTCCTGATAAGGCAAGAGAGTGCCGGCCCCCTCTTGCCCTATCAGGACCTCCACTGCC
ACATAGAGGCCATGATTGACCCTTAGACAAAGGGCTGGTGTCCAATCCCAGCCCCCAGCCCCAG
AACTCCAGGGAATGAATGGGCAGAGAGCAGGAATGTGGGACATCTGTGTTCAAGGGAAGGACTC
CAGGAGTCTGCTGGGAATGAGGCCTAGTAGGAAATGAGGTGGCCCTTGAGGGTACAGAACAGGT
TCATTCTTCGCCAAATTCCCAGCACCTTGCAGGCACTTACAGCTGAGTGAGATAATGCCTGGGT
TATGAAATCAAAAAGTTGGAAAGCAGGTCAGAGGTCATCTGGTACAGCCCTTCCTTCCCTTTTT
TTTTTTTTTTTTTTGTGAGACAAGGTCTCTCTCTGTTGCCCAGGCTGGAGTGGCGCAAACACAG
CTCACTGCAGCCTCAACCTACTGGGCTCAAGCAATCCTCCAGCCTCAGCCTCCCAAAGTGCTGG
GATTACAAGCATGAGCCACCCCACTCAGCCCTTTCCTTCCTTTTTAATTGATGCATAATAATTG
TAAGTATTCATCATGGTCCAACCAACCCTTTCTTGACCCACCTTCCTAGAGAGAGGGTCCTCTT
GATTCAGCGGTCAGGGCCCCAGACCCATGGTCTGGCTCCAGGTACCACCTGCCTCATGCAGGAG
TTGGCGTGCCCAGGAAGCTCTGCCTCTGGGCACAGTGACCTCAGTGGGGTGAGGGGAGCTCTCC
CCATAGCTGGGCTGCGGCCCAACCCCACCCCCTCAGGCTATGCCAGGGGGTGTTGCCAGGGGCA
CCCGGGCATCGCCAGTCTAGCCCACTCCTTCATAAAGCCCTCGCATCCCAGGAGCGAGCAGAGC
CAGAGCAT
Mouse Aldh1L1 promoter
SEQ ID NO: 7
AACTGAGAGTGGAGGGGCACAGAAGAGCCCAAGAGGCTCCTTAGGTTGTGTGGAGGGTACAATA
TGTTTGGGCTGAGCAACCCAGAGCCAGACTTTGTCTGGCTGGTAAGAGACAGAGGTGCCTGCTA
TCACAATCCAAGGGTCTGCTTGAGGCAGAGCCAGTGCAAAGGATGTGGTTAGAGCCAGCCTGGT
GTACTGAAGAGGGGCGAAGAGCTTGAGTAAGGAGTCTCAGCGGTGGTTTGAGAGGCAGGGTGGT
TAATGGAGTAGCTGCAGGGGAGAATCCTTGGGAGGGAGCCTGCAGGACAGAGCTTTGGTCAGGA
AGTGATGGGCATGTCACTGGACCCTGTATTGTCTCTGACTTTTCTCAAGTAGGACAATGACTCT
GCCCAGGGAGGGGGTCTGTGACAAGGTGGAAGGGCCAGAGGAGAACTTCTGAGAAGAAAACCAG
AGGCCGTGAAGAGGTGGGAAGGGCATGGGATTCAGAACCTCAGGCCCACCAGGACACAACCCCA
GGTCCACAGCAGATGGGTGACCTTGCATGTCTCAGTCACCAGCATTGTGCTCCTTGCTTATCAC
GCTTGGGTGAAGGAAATGACCCAAATAGCATAAAGCCTGAAGGCCGGGACTAGGCCAGCTAGGG
CTTGCCCTTCCCTTCCCAGCTGCACTTTCCATAGGTCCCACCTTCAGCAGATTAGACCCGCCTC
CTGCTTCCTGCCTCCTTGCCTCCTCACTCATGGGTCTATGCCCACCTCCAGTCTCGGGACTGAG
GCTCACTGAAGTCCCATCGAGGTCTGGTCTGGTGAATCAGCGGCTGGCTCTGGGCCCTGGGCGA
CCAGTTAGGTTCCGGGCATGCTAGGCAATGAACTCTACCCGGAATTGGGGGTGCGGGGAGGCGG
GGAGGTCTCCAACCCAGCCTTTTGAGGACGTGCCTGTCGCTGCACGGTGCTTTTTATAGACGAT
GGTGGCCCATTTTGCAGAAGGGAAAGCCGGAGCCCTCTGGGGAGCAAGGTCCCCGCAAATGGAC
GGATGACCTGAGCTTGGTTCTGCCAGTCCACTTCCCAAATCCCTCACCCCATTCTAGGGACTAG
GGAAAGATCTCCTGATTGGTCATATCTGGGGGCCTGGCCGGAGGGCCTCCTATGATTGGAGAGA
TCTAGGCTGGGCGGGCCCTAGAGCCCGCCTCTTCTCTGCCTGGAGGAGGAGCACTGACCCTAAC
CCTCTCTGCACAAGACCCGAGCTTGTGCGCCCTTCTGGGAGCTTGCTGCCCCTGTGCTGACTGC
TGACAGCTGACTGACGCTCGCAGCTAGCAGGTACTTCTGGGTTGCTAGCCCAGAGCCCTGGGCC
GGTGACCCTGTTTTCCCTACTTCCCGTCTTTGACCTTGGGTAAGTTTCTTTTTCTTTTGTTTTT
GAGAGAGGCACCCAGATCCTCTCCACTACAGGCAGCCGCTGAACCTTGGATCCTCAGCTCCTGC
CCTGGGAACTACAGTTCCTGCCCTTTTTTTCCCACCTTGAGGGAGGTTTTCCCTGAGTAGCTTC
GACTATCCTGGAACAAGCTTTGTAGACCAGCCTGGGTCTCCGGAGAGTTGGGATTAAAGGCGTG
CACCACCACC
Human NG2 promoter
SEQ ID NO: 8
CTCTGGTTTCAAGACCAATACTCATAACCCCCACATGGACCAGGCACCATCACACCTGAGCACT
GCACTTAGGGTCAAAGACCTGGCCCCACATCTCAGCAGCTATGTAGACTAGCTCCAGTCCCTTA
ATCTCTCTCAGCCTCAGTTTCTTCATCTGCAAAACAGGTCTCAGTTTCGTTGCAAAGTATGAAG
TGCTGGGCTGTTACTGGTCAAAGGGAAGAGCTGGGAAGAGGGTGCAAGGTGGGGTTGGGCTGGA
GATGGGCTGGAGCAGATAGATGGAGGGACCTGAATGGAGGAAGTAAACCAAGGCCCGGTAACAT
TGGGACTGGACAGAGAACACGCAGATCCTCTAGGCACCGGAAGCTAAGTAACATTGCCCTTTCT
CCTCCTGTTTGGGACTAGGCTGATGTTGCTGCCTGGAAGGGAGCCAGCAGAAGGGCCCCAGCCT
GAAGCTGTTAGGTAGAAGCCAAATCCAGGGCCAGATTTCCAGGAGGCAGCCTCGGGAAGTTGAA
ACACCCGGATTCAGGGGTCAGGAGGCCTGGGCTTCTGGCACCAAACGGCCAGGGACCTACTTTC
CACCTGGAGTCTTGTAAGAGCCACTTTCAGCTTGAGCTGCACTTTCGTCCTCCATGAAATGGGG
GAGGGGATGCTCCTCACCCACCTTGCAAGGTTATTTTGAGGCAAATGTCATGGCGGGACTGAGA
ATTCTTCTGCCCTGCGAGGAAATCCAGACATCTCTCCCTTACAGACAGGGAGACTGAGGTGAGG
CCCTTCCAGGCAGAGAAGGTCACTGTTGCAGCCATGGGCAGTGCCCCACAGGACCTCGGGTGGT
GCCTCTGGAGTCTGGAGAAGTTCCTAGGGGACCTCCGAGGCAAAGCAGCCCAAAAGCCGCCTGT
GAGGGTGGCTGGTGTCTGTCCTTCCTCCTAAGGCTGGAGTGTGCCTGTGGAGGGGTCTCCTGAA
CTCCCGCAAAGGCAGAAAGGAGGGAAGTAGGGGCTGGGACAGTTCATGCCTCCTCCCTGAGGGG
GTCTCCCGGGCTCGGCTCTTGGGGCCAGAGTTCAGGGTGTCTGGGCCTCTCTATGACTTTGTTC
TAAGTCTTTAGGGTGGGGCTGGGGTCTGGCCCAGCTGCAAGGGCCCCCTCACCCCTGCCCCAGA
GAGGAACAGCCCCGCACGGGCCCTTTAAGAAGGTTGAGGGTGGGGGCAGGTGGGGGAGTCCAAG
CCTGAAACCCGAGCGGGCGCGCGGGTCTGCGCCTGCCCCGCCCCCGGAGTTAAGTGCGCGGACA
CCCGGAGCCGGCCCGCGCCCAGGAGCAGAGCCGCGCTCGCTCCACTCAGCTCCCAGCTCCCAGG
ACTCCGCTGGCTCCTCGCAAGTCCTGCCGCCCAGCCCGCCGGG
CAG::NeuroD1-IRES-GFP
SEQ ID NO: 9
GATCCGGCCATTAGCCATATTATTCATTGGTTATATAGCATAAATCAATATTGGCTATTGGCCA
TTGCATACGTTGTATCCATATCATAATATGTACATTTATATTGGCTCATGTCCAACATTACCGC
CATGTTGACATTGATTATTGACTAGTTATTAATAGTAATCAATTACGGGGTCATTAGTTCATAG
CCCATATATGGAGTTCCGCGTTACATAACTTACGGTAAATGGCCCGCCTGGCTGACCGCCCAAC
GACCCCCGCCCATTGACGTCAATAATGACGTATGTTCCCATAGTAACGCCAATAGGGACTTTCC
ATTGACGTCAATGGGTGGAGTATTTACGGTAAACTGCCCACTTGGCAGTACATCAAGTGTATCA
TATGCCAAGTACGCCCCCTATTGACGTCAATGACGGTAAATGGCCCGCCTGGCATTATGCCCAG
TACATGACCTTATGGGACTTTCCTACTTGGCAGTACATCTACGTATTAGTCATCGCTATTACCA
TGGTGATGCGGTTTTGGCAGTACATCAATGGGCGTGGATAGCGGTTTGACTCACGGGGATTTCC
AAGTCTCCACCCCATTGACGTCAATGGGAGTTTGTTTTGGCACCAAAATCAACGGGACTTTCCA
AAATGTCGTAACAACTCCGCCCCATTGACGCAAATGGGCGGTAGGCATGTACGGTGGGAGGTCT
ATATAAGCAGAGCTCAATAAAAGAGCCCACAACCCCTCACTCGGGGCGCCAGTCCTCCGATTGA
CTGAGTCGCCCGGGTACCCGTATTCCCAATAAAGCCTCTTGCTGTTTGCATCCGAATCGTGGTC
TCGCTGTTCCTTGGGAGGGTCTCCTCTGAGTGATTGACTACCCACGACGGGGGTCTTTCATTTG
GGGGCTCGTCCGGGATTTGGAGACCCCTGCCCAGGGACCACCGACCCACCACCGGGAGGTAAGC
TGGCCAGCAACTTATCTGTGTCTGTCCGATTGTCTAGTGTCTATGTTTGATGTTATGCGCCTGC
GTCTGTACTAGTTAGCTAACTAGCTCTGTATCTGGCGGACCCGTGGTGGAACTGACGAGTTCTG
AACACCCGGCCGCAACCCTGGGAGACGTCCCAGGGACTTTGGGGGCCGTTTTTGTGGCCCGACC
TGAGGAAGGGAGTCGATGTGGAATCCGACCCCGTCAGGATATGTGGTTCTGGTAGGAGACGAGA
ACCTAAAACAGTTCCCGCCTCCGTCTGAATTTTTGCTTTCGGTTTGGAACCGAAGCCGCGCGTC
TTGTCTGCTGCAGCGCTGCAGCATCGTTCTGTGTTGTCTCTGTCTGACTGTGTTTCTGTATTTG
TCTGAAAATTAGGGCCAGACTGTTACCACTCCCTTAAGTTTGACCTTAGGTCACTGGAAAGATG
TCGAGCGGATCGCTCACAACCAGTCGGTAGATGTCAAGAAGAGACGTTGGGTTACCTTCTGCTC
TGCAGAATGGCCAACCTTTAACGTCGGATGGCCGCGAGACGGCACCTTTAACCGAGACCTCATC
ACCCAGGTTAAGATCAAGGTCTTTTCACCTGGCCCGCATGGACACCCAGACCAGGTCCCCTACA
TCGTGACCTGGGAAGCCTTGGCTTTTGACCCCCCTCCCTGGGTCAAGCCCTTTGTACACCCTAA
GCCTCCGCCTCCTCTTCCTCCATCCGCCCCGTCTCTCCCCCTTGAACCTCCTCGTTCGACCCCG
CCTCGATCCTCCCTTTATCCAGCCCTCACTCCTTCTCTAGGCGCCGGAATTCGATGTCGACATT
GATTATTGACTAGTTATTAATAGTAATCAATTACGGGGTCATTAGTTCATAGCCCATATATGGA
GTTCCGCGTTACATAACTTACGGTAAATGGCCCGCCTGGCTGACCGCCCAACGACCCCCGCCCA
TTGACGTCAATAATGACGTATGTTCCCATAGTAACGCCAATAGGGACTTTCCATTGACGTCAAT
GGGTGGACTATTTACGGTAAACTGCCCACTTGGCAGTACATCAAGTGTATCATATGCCAAGTAC
GCCCCCTATTGACGTCAATGACGGTAAATGGCCCGCCTGGCATTATGCCCAGTACATGACCTTA
TGGGACTTTCCTACTTGGCAGTACATCTACGTATTAGTCATCGCTATTACCATGGGTCGAGGTG
AGCCCCACGTTCTGCTTCACTCTCCCCATCTCCCCCCCCTCCCCACCCCCAATTTTGTATTTAT
TTATTTTTTAATTATTTTGTGCAGCGATGGGGGCGGGGGGGGGGGGGGCGCGCGCCAGGCGGGG
CGGGGCGGGGCGAGGGGCGGGGCGGGGCGAGGCGGAGAGGTGCGGCGGCAGCCAATCAGAGCGG
CGCGCTCCGAAAGTTTCCTTTTATGGCGAGGCGGCGGCGGCGGCGGCCCTATAAAAAGCGAAGC
GCGCGGCGGGCGGGAGTCGCTGCGTTGCCTTCGCCCCGTGCCCCGCTCCGCGCCGCCTCGCGCC
GCCCGCCCCGGCTCTGACTGACCGCGTTACTCCCACAGGTGAGCGGGCGGGACGGCCCTTCTCC
TCCGGGCTGTAATTAGCGCTTGGTTTAATGACGGCTCGTTTCTTTTCTGTGGCTGCGTGAAAGC
CTTAAAGGGCTCCGGGAGGGCCCTTTGTGCGGGGGGGAGCGGCTCGGGGGGTGCGTGCGTGTGT
GTGTGCGTGGGGAGCGCCGCGTGCGGCCCGCGCTGCCCGGCGGCTGTGAGCGCTGCGGGCGCGG
CGCGGGGCTTTGTGCGCTCCGCGTGTGCGCGAGGGGAGCGCGGCCGGGGGCGGTGCCCCGCGGT
GCGGGGGGGCTGCGAGGGGAACAAAGGCTGCGTGCGGGGTGTGTGCGTGGGGGGGTGAGCAGGG
GGTGTGGGCGCGGCGGTCGGGCTGTAACCCCCCCCTGCACCCCCCTCCCCGAGTTGCTGAGCAC
GGCCCGGCTTCGGGTGCGGGGCTCCGTGCGGGGCGTGGCGCGGGGCTCGCCGTGCCGGGCGGGG
GGTGGCGGCAGGTGGGGGTGCCGGGCGGGGCGGGGCCGCCTCGGGCCGGGGAGGGCTCGGGGGA
GGGGCGCGGCGGCCCCGGAGCGCCGGCGGCTGTCGAGGCGCGGCGAGCCGCAGCCATTGCCTTT
TATGGTAATCGTGCGAGAGGGCGCAGGGACTTCCTTTGTCCCAAATCTGGCGGAGCCGAAATCT
GGGAGGCGCCGCCGCACCCCCTCTAGCGGGCGCGGGCGAAGCGGTGCGGCGCCGGCAGGAAGGA
AATGGGCGGGGAGGGCCTTCGTGCGTCGCCGCGCCGCCGTCCCCTTCTCCATCTCCAGCCTCGG
GGCTGCCGCAGGGGGACGGCTGCCTTCGGGGGGGACGGGGCAGGGCGGGGTTCGGCTTCTGGCG
TGTGACCGGCGGCTCTAGAGCCTCTGCTAACCATGTTCATGCCTTCTTCTTTTTCCTACAGCTC
CTGGGCAACGTGCTGGTTGTTGTGCTGTCTCATCATTTTGGCAAAGAATTCGCTAGCGGATCCG
GCCGCCTCGGCCACCGGTCGCCACCATCGCCACCATGACCAAATCATACAGCGAGAGCGGGCTG
ATGGGCGAGCCTCAGCCCCAAGGTCCCCCAAGCTGGACAGATGAGTGTCTCAGTTCTCAGGACG
AGGAACACGAGGCAGACAAGAAAGAGGACGAGCTTGAAGCCATGAATGCAGAGGAGGACTCTCT
GAGAAACGGGGGAGAGGAGGAGGAGGAAGATGAGGATCTAGAGGAAGAGGAGGAAGAAGAAGAG
GAGGAGGAGGATCAAAAGCCCAAGAGACGGGGTCCCAAAAAGAAAAAGATGACCAAGGCGCGCC
TAGAACGTTTTAAATTAAGGCGCATGAAGGCCAACGCCCGCGAGCGGAACCGCATGCACGGGCT
GAACGCGGCGCTGGACAACCTGCGCAAGGTGGTACCTTGCTACTCCAAGACCCAGAAACTGTCT
AAAATAGAGACACTGCGCTTGGCCAAGAACTACATCTGGGCTCTGTCAGAGATCCTGCGCTCAG
GCAAAAGCCCTGATCTGGTCTCCTTCGTACAGACGCTCTGCAAAGGTTTGTCCCAGCCCACTAC
CAATTTGGTCGCCGGCTGCCTGCAGCTCAACCCTCGGACTTTCTTGCCTGAGCAGAACCCGGAC
ATGCCCCCGCATCTGCCAACCGCCAGCGCTTCCTTCCCGGTGCATCCCTACTCCTACCAGTCCC
CTGGACTGCCCAGCCCGCCCTACGGCACCATGGACAGCTCCCACGTCTTCCACGTCAAGCCGCC
GCCACACGCCTACAGCGCAGCTCTGGAGCCCTTCTTTGAAAGCCCCCTAACTGACTGCACCAGC
CCTTCCTTTGACGGACCCCTCAGCCCGCCGCTCAGCATCAATGGCAACTTCTCTTTCAAACACG
AACCATCCGCCGAGTTTGAAAAAAATTATGCCTTTACCATGCACTACCCTGCAGCGACGCTGGC
AGGGCCCCAAAGCCACGGATCAATCTTCTCTTCCGGTGCCGCTGCCCCTCGCTGCGAGATCCCC
ATAGACAACATTATGTCTTTCGATAGCCATTCGCATCATGAGCGAGTCATGAGTGCCCAGCTTA
ATGCCATCTTTCACGATTAGGTTTAAACGCGGCCGCGCCCCTCTCCCTCCCCCCCCCCTAACGT
TACTGGCCGAAGCCGCTTGGAATAAGGCCGGTGTGCGTTTGTCTATATGTTATTTTCCACCATA
TTGCCGTCTTTTGGCAATGTGAGGGCCCGGAAACCTGGCCCTGTCTTCTTGACGAGCATTCCTA
GGGGTCTTTCCCCTCTCGCCAAAGGAATGCAAGGTCTGTTGAATGTCGTGAAGGAAGCAGTTCC
TCTGGAAGCTTCTTGAAGACAAACAACGTCTGTAGCGACCCTTTGCAGGCAGCGGAACCCCCCA
CCTGGCGACAGGTGCCTCTGCGGCCAAAAGCCACGTGTATAAGATACACCTGCAAAGGCGGCAC
AACCCCAGTGCCACGTTGTGAGTTGGATAGTTGTGGAAAGAGTCAAATGGCTCTCCTCAAGCGT
ATTCAACAAGGGGCTGAAGGATGCCCAGAAGGTACCCCATTGTATGGGATCTGATCTGGGGCCT
CGGTGCACATGCTTTACATGTGTTTAGTCGAGGTTAAAAAAACGTCTAGGCCCCCCGAACCACG
GGGACGTGGTTTTCCTTTGAAAAACACGATGATAATATGGCCACAACCATGGTGAGCAAGGGCG
AGGAGCTGTTCACCGGGGTGGTGCCCATCCTGGTCGAGCTGGACGGCGACGTAAACGGCCACAA
GTTCAGCGTGTCCGGCGAGGGCGAGGGCGATGCCACCTACGGCAAGCTGACCCTGAAGTTCATC
TGCACCACCGGCAAGCTGCCCGTGCCCTGGCCCACCCTCGTGACCACCCTGACCTACGGCGTGC
AGTGCTTCAGCCGCTACCCCGACCACATGAAGCAGCACGACTTCTTCAAGTCCGCCATGCCCGA
AGGCTACGTCCAGGAGCGCACCATCTTCTTCAAGGACGACGGCAACTACAAGACCCGCGCCGAG
GTGAAGTTCGAGGGCGACACCCTGGTGAACCGCATCGAGCTGAAGGGCATCGACTTCAAGGAGG
ACGGCAACATCCTGGGGCACAAGCTGGAGTACAACTACAACAGCCACAACGTCTATATCATGGC
CGACAAGCAGAAGAACGGCATCAAGGTGAACTTCAAGATCCGCCACAACATCGAGGACGGCAGC
GTGCAGCTCGCCGACCACTACCAGCAGAACACCCCCATCGGCGACGGCCCCGTGCTGCTGCCCG
ACAACCACTACCTGAGCACCCAGTCCGCCCTGAGCAAAGACCCCAACGAGAAGCGCGATCACAT
GGTCCTGCTGGAGTTCGTGACCGCCGCCGGGATCACTCTCGGCATGGACGAGCTGTACAAGTAA
GTCGACAATCAACCTCTGGATTACAAAATTTGTGAAAGATTGACTGGTATTCTTAACTATGTTG
CTCCTTTTACGCTATGTGGATACGCTGCTTTAATGCCTTTGTATCATGCTATTGCTTCCCGTAT
GGCTTTCATTTTCTCCTCCTTGTATAAATCCTGGTTGCTGTCTCTTTATGAGGAGTTGTGGCCC
GTTGTCAGGCAACGTGGCGTGGTGTGCACTGTGTTTGCTGACGCAACCCCCACTGGTTGGGGCA
TTGCCACCACCTGTCAGCTCCTTTCCGGGACTTTCGCTTTCCCCCTCCCTATTGCCACGGCGGA
ACTCATCGCCGCCTGCCTTGCCCGCTGCTGGACAGGGGCTCGGCTGTTGGGCACTGACAATTCC
GTGGTGTTGTCGGGGAAGCTGACGTCCTTTCCATGGCTGCTCGCCTGTGTTGCCACCTGGATTC
TGCGCGGGACGTCCTTCTGCTACGTCCCTTCGGCCCTCAATCCAGCGGACCTTCCTTCCCGCGG
CCTGCTGCCGGCTCTGCGGCCTCTTCCGCGTCTTCGCCTTCGCCCTCAGACGAGTCGGATCTCC
CTTTGGGCCGCCTCCCCGCCTGGAATTCGAGCTCGAGCTTGTTAACATCGATAAAATAAAAGAT
TTTATTTAGTCTCCAGAAAAAGGGGGGAATGAAAGACCCCACCTGTAGGTTTGGCAAGCTAGCT
TAAGTAACGCCATTTTGCAAGGCATGGAAAAATACATAACTGAGAATAGAGAAGTTCAGATCAA
GGTCAGGAACAGATGGAACAGCTGAATATGGGCCAAACAGGATATCTGTGGTAAGCAGTTCCTG
CCCCGGCTCAGGGCCAAGAACAGATGGAACAGCTGAATATGGGCCAAACAGGATATCTGTGGTA
AGCAGTTCCTGCCCCGGCTCAGGGCCAAGAACAGATGGTCCCCAGATGCGGTCCAGCCCTCAGC
AGTTTCTAGAGAACCATCAGATGTTTCCAGGGTGCCCCAAGGACCTGAAATGACCCTGTGCCTT
ATTTGAACTAACCAATCAGTTCGCTTCTCGCTTCTGTTCGCGCGCTTCTGCTCCCCGAGCTCAA
TAAAAGAGCCCACAACCCCTCACTCGGGGCGCCAGTCCTCCGATTGACTGAGTCGCCCGGGTAC
CCGTGTATCCAATAAACCCTCTTGCAGTTGCATCCGACTTGTGGTCTCGCTGTTCCTTGGGAGG
GTCTCCTCTGAGTGATTGACTACCCGTCAGCGGGGGTCTTTCATTTCCGACTTGTGGTCTCGCT
GCCTTGGGAGGGTCTCCTCTGAGTGATTGACTACCCGTCAGCGGGGGTCTTCACATGCAGCATG
TATCAAAATTAATTTGGTTTTTTTTCTTAAGTATTTACATTAAATGGCCATAGTTGCATTAATG
AATCGGCCAACGCGCGGGGAGAGGCGGTTTGCGTATTGGCGCTCTTCCGCTTCCTCGCTCACTG
ACTCGCTGCGCTCGGTCGTTCGGCTGCGGCGAGCGGTATCAGCTCACTCAAAGGCGGTAATACG
GTTATCCACAGAATCAGGGGATAACGCAGGAAAGAACATGTGAGCAAAAGGCCAGCAAAAGGCC
AGGAACCGTAAAAAGGCCGCGTTGCTGGCGTTTTTCCATAGGCTCCGCCCCCCTGACGAGCATC
ACAAAAATCGACGCTCAAGTCAGAGGTGGCGAAACCCGACAGGACTATAAAGATACCAGGCGTT
TCCCCCTGGAAGCTCCCTCGTGCGCTCTCCTGTTCCGACCCTGCCGCTTACCGGATACCTGTCC
GCCTTTCTCCCTTCGGGAAGCGTGGCGCTTTCTCATAGCTCACGCTGTAGGTATCTCAGTTCGG
TGTAGGTCGTTCGCTCCAAGCTGGGCTGTGTGCACGAACCCCCCGTTCAGCCCGACCGCTGCGC
CTTATCCGGTAACTATCGTCTTGAGTCCAACCCGGTAAGACACGACTTATCGCCACTGGCAGCA
GCCACTGGTAACAGGATTAGCAGAGCGAGGTATGTAGGCGGTGCTACAGAGTTCTTGAAGTGGT
GGCCTAACTACGGCTACACTAGAAGGACAGTATTTGGTATCTGCGCTCTGCTGAAGCCAGTTAC
CTTCGGAAAAAGAGTTGGTAGCTCTTGATCCGGCAAACAAACCACCGCTGGTAGCGGTGGTTTT
TTTGTTTGCAAGCAGCAGATTACGCGCAGAAAAAAAGGATCTCAAGAAGATCCTTTGATCTTTT
CTACGGGGTCTGACGCTCAGTGGAACGAAAACTCACGTTAAGGGATTTTGGTCATGAGATTATC
AAAAAGGATCTTCACCTAGATCCTTTTAAATTAAAAATGAAGTTTGCGGCCGGCCGCAAATCAA
TCTAAAGTATATATGAGTAAACTTGGTCTGACAGTTACCAATGCTTAATCAGTGAGGCACCTAT
CTCAGCGATCTGTCTATTTCGTTCATCCATAGTTGCCTGACTCCCCGTCGTGTAGATAACTACG
ATACGGGAGGGCTTACCATCTGGCCCCAGTGCTGCAATGATACCGCGAGACCCACGCTCACCGG
CTCCAGATTTATCAGCAATAAACCAGCCAGCCGGAAGGGCCGAGCGCAGAAGTGGTCCTGCAAC
TTTATCCGCCTCCATCCAGTCTATTAATTGTTGCCGGGAAGCTAGAGTAAGTAGTTCGCCAGTT
AATAGTTTGCGCAACGTTGTTGCCATTGCTACAGGCATCGTGGTGTCACGCTCGTCGTTTGGTA
TGGCTTCATTCAGCTCCGGTTCCCAACGATCAAGGCGAGTTACATGATCCCCCATGTTGTGCAA
AAAAGCGGTTAGCTCCTTCGGTCCTCCGATCGTTGTCAGAAGTAAGTTGGCCGCAGTGTTATCA
CTCATGGTTATGGCAGCACTGCATAATTCTCTTACTGTCATGCCATCCGTAAGATGCTTTTCTG
TGACTGGTGAGTACTCAACCAAGTCATTCTGAGAATAGTGTATGCGGCGACCGAGTTGCTCTTG
CCCGGCGTCAACACGGGATAATACCGCGCCACATAGCAGAACTTTAAAAGTGCTCATCATTGGA
AAACGTTCTTCGGGGCGAAAACTCTCAAGGATCTTACCGCTGTTGAGATCCAGTTCGATGTAAC
CCACTCGTGCACCCAACTGATCTTCAGCATCTTTTACTTTCACCAGCGTTTCTGGGTGAGCAAA
AACAGGAAGGCAAAATGCCGCAAAAAAGGGAATAAGGGCGACACGGAAATGTTGAATACTCATA
CTCTTCCTTTTTCAAT
Human Dlx2 nucleic acid sequence encoding human Dlx2 protein
SEQ ID NO: 10
ATGACTGGAGTCTTTGACAGTCTAGTGGCTGATATGCACTCGACCCAGATCGCCGCCTCCAGCA
CGTACCACCAGCACCAGCAGCCCCCGAGCGGCGGCGGCGCCGGCCCGGGTGGCAACAGCAGCAG
CAGCAGCAGCCTCCACAAGCCCCAGGAGTCGCCCACCCTTCCGGTGTCCACCGCCACCGACAGC
AGCTACTACACCAACCAGCAGCACCCGGCGGGCGGCGGCGGCGGCGGGGGCTCGCCCTACGCGC
ACATGGGTTCCTACCAGTACCAAGCCAGCGGCCTCAACAACGTCCCTTACTCCGCCAAGAGCAG
CTATGACCTGGGCTACACCGCCGCCTACACCTCCTACGCTCCCTATGGAACCAGTTCGTCCCCA
GCCAACAACGAGCCTGAGAAGGAGGACCTTGAGCCTGAAATTCGGATAGTGAACGGGAAGCCAA
AGAAAGTCCGGAAACCCCGCACCATCTACTCCAGTTTCCAGCTGGCGGCTCTTCAGCGGCGTTT
CCAAAAGACTCAATACTTGGCCTTGCCGGAGCGAGCCGAGCTGGCGGCCTCTCTGGGCCTCACC
CAGACTCAGGTCAAAATCTGGTTCCAGAACCGCCGGTCCAAGTTCAAGAAGATGTGGAAAAGTG
GTGAGATCCCCTCGGAGCAGCACCCTGGGGCCAGCGCTTCTCCACCTTGTGCTTCGCCGCCAGT
CTCAGCGCCGGCCTCCTGGGACTTTGGTGTGCCGCAGCGGATGGCGGGCGGCGGTGGTCCGGGC
AGTGGCGGCAGCGGCGCCGGCAGCTCGGGCTCCAGCCCGAGCAGCGCGGCCTCGGCTTTTCTGG
GCAACTACCCCTGGTACCACCAGACCTCGGGATCCGCCTCACACCTGCAGGCCACGGCGCCGCT
GCTGCACCCCACTCAGACCCCGCAGCCGCATCACCACCACCACCATCACGGCGGCGGGGGCGCC
CCGGTGAGCGCGGGGACGATTTTCTAA
Human Dlx2 amino acid sequence - encoded by SEQ ID NO: 10
SEQ ID NO: 11
MTGVFDSLVADMHSTQIAASSTYHQHQQPPSGGGAGPGGNSSSSSSLHKPQESPTLPVSTATDS
SYYTNQQHPAGGGGGGGSPYAHMGSYQYQASGLNNVPYSAKSSYDLGYTAAYTSYAPYGTSSSP
ANNEPEKEDLEPEIRIVNGKPKKVRKPRTIYSSFQLAALQRRFQKTQYLALPERAELAASLGLT
QTQVKIWFQNRRSKFKKMWKSGEIPSEQHPGASASPPCASPPVSAPASWDFGVPQRMAGGGGPG
SGGSGAGSSGSSPSSAASAFLGNYPWYHQTSGSASHLQATAPLLHPTQTPQPHHHHHHHGGGGA
PVSAGTIF
Mouse Dlx2 nucleic acid sequence encoding mouse Dlx2 protein
SEQ ID NO: 12
ATGACTGGAGTCTTTGACAGTCTGGTGGCTGATATGCACTCGACCCAGATCACCGCCTCCAGCA
CGTACCACCAGCACCAGCAGCCCCCGAGCGGTGCGGGCGCCGGCCCTGGCGGCAACAGCAACAG
CAGCAGCAGCAACAGCAGCCTGCACAAGCCCCAGGAGTCGCCAACCCTCCCGGTGTCCACGGCT
ACGGACAGCAGCTACTACACCAACCAGCAGCACCCGGCGGGCGGCGGCGGCGGGGGGGCCTCGC
CCTACGCGCACATGGGCTCCTACCAGTACCACGCCAGCGGCCTCAACAATGTCTCCTACTCCGC
CAAAAGCAGCTACGACCTGGGCTACACCGCCGCGTACACCTCCTACGCGCCCTACGGCACCAGT
TCGTCTCCGGTCAACAACGAGCCGGACAAGGAAGACCTTGAGCCTGAAATCCGAATAGTGAACG
GGAAGCCAAAGAAAGTCCGGAAACCACGCACCATCTACTCCAGTTTCCAGCTGGCGGCCCTTCA
ACGACGCTTCCAGAAGACCCAGTATCTGGCCCTGCCAGAGCGAGCCGAGCTGGCGGCGTCCCTG
GGCCTCACCCAAACTCAGGTCAAAATCTGGTTCCAGAACCGCCGATCCAAGTTCAAGAAGATGT
GGAAAAGCGGCGAGATACCCACCGAGCAGCACCCTGGAGCCAGCGCTTCTCCTCCTTGTGCCTC
CCCGCCGGTCTCGGCGCCAGCATCCTGGGACTTCGGCGCGCCGCAGCGGATGGCTGGCGGCGGC
CCGGGCAGCGGAGGCGGCGGTGCGGGCAGCTCTGGCTCCAGCCCGAGCAGCGCCGCCTCGGCCT
TTCTGGGAAACTACCCGTGGTACCACCAGGCTTCGGGCTCCGCTTCACACCTGCAGGCCACAGC
GCCACTTCTGCATCCTTCGCAGACTCCGCAGGCGCACCATCACCACCATCACCACCACCACGCA
GGCGGGGGCGCCCCGGTGAGCGCGGGGACGATTTTCTAA
Mouse Dlx2 amino acid sequence - encoded by SEQ ID NO: 12
SEQ ID NO: 13
MTGVFDSLVADMHSTQITASSTYHQHQQPPSGAGAGPGGNSNSSSSNSSLHKPQESPTLPVSTA
TDSSYYTNQQHPAGGGGGGASPYAHMGSYQYHASGLNNVSYSAKSSYDLGYTAAYTSYAPYGTS
SSPVNNEPDKEDLEPEIRIVNGKPKKVRKPRTIYSSFQLAALQRRFQKTQYLALPERAELAASL
GLTQTQVKIWFQNRRSKFKKMWKSGEIPTEQHPGASASPPCASPPVSAPASWDFGAPQRMAGGG
PGSGGGGAGSSGSSPSSAASAFLGNYPWYHQASGSASHLQATAPLLHPSQTPQAHHHHHHHHHA
GGGAPVSAGTIF
Human Isl1 nucleic acid sequence encoding human
Isl1 protein
SEQ ID NO: 14
tgaaggaaga ggaagaggag gagagggagg ccagagccag aacagcccgg cagcccgggc
ttcgggggag aacggcctga gccccgagca agttgcctcg ggagccctaa tcctctcccg
ctggctcgcc gagcggtcag tggcgctcag cggcggcgag gctgaaatat gataatcaga
acagctgcgc cgcgcgccct gcagccaatg ggcgcggcgc tcgcctgacg tccccgcgcg
ctgcgtcaga ccaatggcga tggagctgag ttggagcaga gaagtttgag taagagataa
ggaagagagg tgcccgagcc gcgccgagtc tgccgccgcc gcagcgcctc cgctccgcca
actccgccgg cttaaattgg aatcctagat ccgcgagggc gcggcgcagc cgagcagcgg
ctctttcagc attggcaacc ccaggggcca atatttccca cttagccaca gctccagcat
cctctctgtg ggctgttcac cagctgtaca accaccattt cactgtggac attactccct
cttacagata tgggagacat gggagatcca ccaaaaaaaa aacgtctgat ttccctatgt
gttggttgcg gcaatcagat tcacgatcag tatattctga gggtttctcc ggatttggaa
tggcatgcgg catgtttgaa atgtgcggag tgtaatcagt atttggacga gagctgtaca
tgctttgtta gggatgggaa aacctactgt aaaagagatt atatcaggtt gtacgggatc
aaatgcgcca agtgcagcat cggcttcagc aagaacgact tcgtgatgcg tgcccgctcc
aaggtgtatc acatcgagtg tttccgctgt gtggcctgca gccgccagct catccctggg
gacgaatttg cgcttcggga ggacggtctc ttctgccgag cagaccacga tgtggtggag
agggccagtc taggcgctgg cgacccgctc agtcccctgc atccagcgcg gccactgcaa
atggcagcgg agcccatctc cgccaggcag ccggccctgc ggccccacgt ccacaagcag
ccggagaaga ccacccgcgt gcggactgtg ctgaacgaga agcagctgca caccttgcgg
acctgctacg ccgcaaaccc gcggccagat gcgctcatga aggagcaact ggtagagatg
acgggcctca gtccccgtgt gatccgggtc tggtttcaaa acaagcggtg caaggacaag
aagcgaagca tcatgatgaa gcaactccag cagcagcagc ccaatgacaa aactaatatc
caggggatga caggaactcc catggtggct gccagtccag agagacacga cggtggctta
caggctaacc cagtggaagt acaaagttac cagccacctt ggaaagtact gagcgacttc
gccttgcaga gtgacataga tcagcctgct tttcagcaac tggtcaattt ttcagaagga
ggaccgggct ctaattccac tggcagtgaa gtagcatcaa tgtcctctca acttccagat
acacctaaca gcatggtagc cagtcctatt gaggcatgag gaacattcat tctgtatttt
ttttccctgt tggagaaagt gggaaattat aatgtcgaac tctgaaacaa aagtatttaa
cgacccagtc aatgaaaact gaatcaagaa atgaatgctc catgaaatgc acgaagtctg
ttttaatgac aaggtgatat ggtagcaaca ctgtgaagac aatcatggga ttttactaga
attaaacaac aaacaaaacg caaaacccag tatatgctat tcaatgatct tagaagtact
gaaaaaaaaa gacgttttta aaacgtagag gatttatatt caaggatctc aaagaaagca
ttttcatttc actgcacatc tagagaaaaa caaaaataga aaattttcta gtccatccta
atctgaatgg tgctgtttct atattggtca ttgccttgcc aaacaggagc tccagcaaaa
gcgcaggaag agagactggc ctccttggct gaaagagtcc tttcaggaag gtggagctgc
attggtttga tatgtttaaa gttgacttta acaaggggtt aattgaaatc ctgggtctct
tggcctgtcc tgtagctggt ttatttttta ctttgccccc tccccacttt ttttgagatc
catcctttat caagaagtct gaagcgactt taaaggtttt tgaattcaga tttaaaaacc
aacttataaa gcattgcaac aaggttacct ctattttgcc acaagcgtct cgggattgtg
tttgacttgt gtctgtccaa gaacttttcc cccaaagatg tgtatagtta ttggttaaaa
tgactgtttt ctctctctat ggaaataaaa aggaaaaaaa aaaaaaaa
Human Isl1 amino acid sequence - encoded by SEQ ID NO: 14
SEQ ID NO: 15
MGDMGDPPKKKRLISLCVGCGNQIHDQYILRVSPDLEWHAACLKCAECNQYLDESCTCFVRDGK
TYCKRDYIRLYGIKCAKCSIGFSKNDFVMRARSKVYHIECFRCVACSRQLIPGDEFALREDGLF
CRADHDVVERASLGAGDPLSPLHPARPLQMAAEPISARQPALRPHVHKQPEKTTRVRTVLNEKQ
LHTLRTCYAANPRPDALMKEQLVEMTGLSPRVIRVWFQNKRCKDKKRSIMMKQLQQQQPNDKTN
IQGMTGTPMVAASPERHDGGLQANPVEVQSYQPPWKVLSDFALQSDIDQPAFQQLVNFSEGGPG
SNSTGSEVASMSSQLPDTPNSMVASPIEA
Mouse Isl1 nucleic acid sequence encoding mouse
Isl1 protein
SEQ ID NO: 16
caactccgcc ggcttaaatc ggactcccag atctgcgagg gcgcggcgca gccgggcagc
tgtttccccc agttttggca accccggggg ccactatttg ccacctagcc acagcaccag
catcctctct gtgggctatt caccaattgt ccaaccacca tttcactgtg gacgttactc
cctcttacag atatgggaga catgggcgat ccaccaaaaa aaaaacgtct gatttccctg
tgtgttggtt gcggcaatca aattcacgac cagtatattc tgagggtttc tccggatttg
gagtggcatg cagcatgttt gaaatgtgcg gagtgtaatc agtatttgga cgaaagctgt
acgtgctttg ttagggatgg gaaaacctac tgtaaaagag attatatcag gttgtacggg
atcaaatgcg ccaagtgcag cataggcttc agcaagaacg acttcgtgat gcgcgcccgc
tctaaggtgt accacatcga gtgtttccgc tgtgtagcct gcagccgaca gctcatcccg
ggagacgaat tcgccctgcg ggaggatggg cttttctgcc gtgcagacca cgatgtggtg
gagagagcca gcctgggagc tggagaccct ctcagtccct tgcatccagc gcggcctctg
caaatggcag ccgaacccat ctcggctagg cagccagctc tgcggccgca cgtccacaag
cagccggaga agaccacccg agtgcggact gtgctcaacg agaagcagct gcacaccttg
cggacctgct atgccgccaa ccctcggcca gatgcgctca tgaaggagca actagtggag
atgacgggcc tcagtcccag agtcatccga gtgtggtttc aaaacaagcg gtgcaaggac
aagaaacgca gcatcatgat gaagcagctc cagcagcagc aacccaacga caaaactaat
atccagggga tgacaggaac tcccatggtg gctgctagtc cggagagaca tgatggtggt
ttacaggcta acccagtaga ggtgcaaagt taccagccgc cctggaaagt actgagtgac
ttcgccttgc aaagcgacat agatcagcct gcttttcagc aactggtcaa tttttcagaa
ggaggaccag gctctaattc tactggcagt gaagtagcat cgatgtcctc gcagctccca
gatacaccca acagcatggt agccagtcct attgaggcat gaggaacatt cattcagatg
ttttgttttg ttttgttttg tttttttccc ctgttggaga aagtggg
Mouse Isl1 amino acid sequence - encoded by SEQ ID NO: 16
SEQ ID NO: 17
MGDMGDPPKKKRLISLCVGCGNQIHDQYILRVSPDLEWHAACLKCAECNQYLDESCTCFVRDGK
TYCKRDYIRLYGIKCAKCSIGFSKNDFVMRARSKVYHIECFRCVACSRQLIPGDEFALREDGLF
CRADHDVVERASLGAGDPLSPLHPARPLQMAAEPISARQPALRPHVHKQPEKTTRVRTVLNEKQ
LHTLRTCYAANPRPDALMKEQLVEMTGLSPRVIRVWFQNKRCKDKKRSIMMKQLQQQQPNDKTN
IQGMTGTPMVAASPERHDGGLQANPVEVQSYQPPWKVLSDFALQSDIDQPAFQQLVNFSEGGPG
SNSTGSEVASMSSQLPDTPNSMVASPIEA

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-121. (canceled)

122. A method of increasing the presence of choline acetyltransferase (ChAT) positive motor neurons in a mammal having Amyotrophic Lateral Sclerosis (ALS) in need thereof, wherein the method comprises injecting a composition comprising an exogenous nucleic acid encoding a NeuroD1 polypeptide into a ventral horn of a spinal cord of the mammal, thereby increasing the presence of ChAT positive motor neurons in the central nervous system of the mammal and improving motor function in the mammal.

123. The method of claim 122, wherein the mammal is a human.

124. The method of claim 122, wherein the injecting step comprises delivering an adeno-associated virus (AAV) expression vector of serotype 9 comprising the exogenous nucleic acid encoding the NeuroD1 polypeptide to the ventral horn of the spinal cord.

125. The method of claim 122, wherein the injecting step comprises delivering a recombinant viral expression vector comprising the exogenous nucleic acid encoding the NeuroD1 polypeptide to the ventral horn of the spinal cord.

126. The method of claim 122, wherein the injecting step comprises delivering an adeno-associated virus (AAV) expression vector comprising the exogenous nucleic acid encoding the NeuroD1 polypeptide to the ventral horn of the spinal cord.

127. The method of claim 126, wherein the AAV expression vector comprises a promoter selected from a group consisting of a glial fibrillary acidic protein (GFAP) promoter, an aldehyde dehydrogenase 1 family member L1 (Aldh1L1) promoter, a neuron-glial antigen 2 (NG2) promoter, a lipocalin 2 (lcn2) gene promoter, an S100b promoter, and a CAG promoter.

128. The method of claim 126, wherein the AAV expression vector comprises a glial fibrillary acidic protein (GFAP) promoter.

129. The method of claim 122, wherein the exogenous nucleic acid encoding the NeuroD1 polypeptide comprises SEQ ID NO: 1.

130. The method of claim 124, wherein the exogenous nucleic acid encoding the NeuroD1 polypeptide comprises SEQ ID NO:1.

131. The method of claim 123, wherein the NeuroD1 polypeptide comprises SEQ ID NO: 2.

132. The method of claim 125, wherein the NeuroD1 polypeptide comprises SEQ ID NO: 2.

133. The method of claim 122, wherein the method further comprises administering an exogenous nucleic acid encoding an Isl1 polypeptide.

134. The method of claim 133, wherein the Isl1 polypeptide comprises SEQ ID NO: 15.

135. The method of claim 133, wherein the exogenous nucleic acid encoding the Isl1 polypeptide comprises SEQ ID NO: 14.

136. The method of claim 122, wherein the method further comprises administering an exogenous nucleic acid encoding an Lhx3 polypeptide to the mammal.

137. The method of claim 122, wherein the method further comprises administering an exogenous nucleic acid encoding an Isl1 polypeptide and an exogenous nucleic acid encoding an Lhx3 polypeptide to the mammal.

138. The method of claim 122, wherein the method further comprises administering one or more exogenous nucleic acids encoding Ngn2, mir218, and mir124 to the mammal.

139. A method of increasing the presence of choline acetyltransferase (ChAT) positive motor neurons in a mammal having Amyotrophic lateral sclerosis (ALS) in need thereof, wherein the method comprises injecting a composition comprising (a) an exogenous nucleic acid encoding a human NeuroD1 polypeptide, and (b) an exogenous nucleic acid encoding a human Isl1 polypeptide into a ventral horn of a spinal cord of the mammal, thereby increasing the presence of ChAT positive motor neurons in the central nervous system of the mammal and improving motor function in the mammal.

140. The method of claim 139, wherein the injecting step comprises delivering one or more recombinant adeno-associated virus expression vectors comprising (i) the exogenous nucleic acid encoding the human NeuroD1 polypeptide, and (ii) the exogenous nucleic acid encoding the human Isl1 polypeptide to the ventral horn of the spinal cord.

141. The method of claim 139, wherein the human NeuroD1 polypeptide comprises SEQ ID NO:2, and the human Isl1 polypeptide comprises SEQ ID NO:15.


SEQUENCE LISTING (17 sequences)

SEQ ID NO: 1 - DNA, 1071 bp, Homo sapiens
  source 1..1071: mol_type = unassigned DNA
atgaccaaatcgtacagcgagagtgggctgatgggcgagcctcagccccaaggtcctccaagctggacagacgagtgtctcagttctcaggacgaggagcacgaggcagacaagaaggaggacgacctcgaagccatgaacgcagaggaggactcactgaggaacgggggagaggaggaggacgaagatgaggacctggaagaggaggaagaagaggaagaggaggatgacgatcaaaagcccaagagacgcggccccaaaaagaagaagatgactaaggctcgcctggagcgttttaaattgagacgcatgaaggctaacgcccgggagcggaaccgcatgcacggactgaacgcggcgctagacaacctgcgcaaggtggtgccttgctattctaagacgcagaagctgtccaaaatcgagactctgcgcttggccaagaactacatctgggctctgtcggagatcctgcgctcaggcaaaagcccagacctggtctccttcgttcagacgctttgcaagggcttatcccaacccaccaccaacctggttgcgggctgcctgcaactcaatcctcggacttttctgcctgagcagaaccaggacatgcccccccacctgccgacggccagcgcttccttccctgtacacccctactcctaccagtcgcctgggctgcccagtccgccttacggtaccatggacagctcccatgtcttccacgttaagcctccgccgcacgcctacagcgcagcgctggagcccttctttgaaagccctctgactgattgcaccagcccttcctttgatggacccctcagcccgccgctcagcatcaatggcaacttctctttcaaacacgaaccgtccgccgagtttgagaaaaattatgcctttaccatgcactatcctgcagcgacactggcaggggcccaaagccacggatcaatcttctcaggcaccgctgcccctcgctgcgagatccccatagacaatattatgtccttcgatagccattcacatcatgagcgagtcatgagtgcccagctcaatgccatatttcatgattag

SEQ ID NO: 2 - protein, 356 aa, Homo sapiens
MTKSYSESGLMGEPQPQGPPSWTDECLSSQDEEHEADKKEDDLEAMNAEEDSLRNGGEEEDEDEDLEEEEEEEEEDDDQKPKRRGPKKKKMTKARLERFKLRRMKANARERNRMHGLNAALDNLRKVVPCYSKTQKLSKIETLRLAKNYIWALSEILRSGKSPDLVSFVQTLCKGLSQPTTNLVAGCLQLNPRTFLPEQNQDMPPHLPTASASFPVHPYSYQSPGLPSPPYGTMDSSHVFHVKPPPHAYSAALEPFFESPLTDCTSPSFDGPLSPPLSINGNFSFKHEPSAEFEKNYAFTMHYPAATLAGAQSHGSIFSGTAAPRCEIPIDNIMSFDSHSHHERVMSAQLNAIFHD

SEQ ID NO: 3 - DNA, 1074 bp, Mus musculus
  source 1..1074: mol_type = unassigned DNA
atgaccaaatcatacagcgagagcgggctgatgggcgagcctcagccccaaggtcccccaagctggacagatgagtgtctcagttctcaggacgaggaacacgaggcagacaagaaagaggacgagcttgaagccatgaatgcagaggaggactctctgagaaacgggggagaggaggaggaggaagatgaggatctagaggaagaggaggaagaagaagaggaggaggaggatcaaaagcccaagagacggggtcccaaaaagaaaaagatgaccaaggcgcgcctagaacgttttaaattaaggcgcatgaaggccaacgcccgcgagcggaaccgcatgcacgggctgaacgcggcgctggacaacctgcgcaaggtggtaccttgctactccaagacccagaaactgtctaaaatagagacactgcgcttggccaagaactacatctgggctctgtcagagatcctgcgctcaggcaaaagccctgatctggtctccttcgtacagacgctctgcaaaggtttgtcccagcccactaccaatttggtcgccggctgcctgcagctcaaccctcggactttcttgcctgagcagaacccggacatgcccccgcatctgccaaccgccagcgcttccttcccggtgcatccctactcctaccagtcccctggactgcccagcccgccctacggcaccatggacagctcccacgtcttccacgtcaagccgccgccacacgcctacagcgcagctctggagcccttctttgaaagccccctaactgactgcaccagcccttcctttgacggacccctcagcccgccgctcagcatcaatggcaacttctctttcaaacacgaaccatccgccgagtttgaaaaaaattatgcctttaccatgcactaccctgcagcgacgctggcagggccccaaagccacggatcaatcttctcttccggtgccgctgcccctcgctgcgagatccccatagacaacattatgtctttcgatagccattcgcatcatgagcgagtcatgagtgcccagcttaatgccatctttcacgattag

SEQ ID NO: 4 - protein, 357 aa, Mus musculus
MTKSYSESGLMGEPQPQGPPSWTDECLSSQDEEHEADKKEDELEAMNAEEDSLRNGGEEEEEDEDLEEEEEEEEEEEDQKPKRRGPKKKKMTKARLERFKLRRMKANARERNRMHGLNAALDNLRKVVPCYSKTQKLSKIETLRLAKNYIWALSEILRSGKSPDLVSFVQTLCKGLSQPTTNLVAGCLQLNPRTFLPEQNPDMPPHLPTASASFPVHPYSYQSPGLPSPPYGTMDSSHVFHVKPPPHAYSAALEPFFESPLTDCTSPSFDGPLSPPLSINGNFSFKHEPSAEFEKNYAFTMHYPAATLAGPQSHGSIFSSGAAAPRCEIPIDNIMSFDSHSHHERVMSAQLNAIFHD

SEQ ID NO: 5 - DNA, 1053 bp, Mus musculus
  source 1..1053: mol_type = unassigned DNA
gcagtgtggagacacacccactttccccaagggctcctgctcccccaagtgatccccttatcctccgtgctaagatgacaccgaggttgcagtccttacctttgaaagcagccacaagggcgtgggggtgcacacctttaatcccagcactcgggaggcagaggcaggcagatttctgagttcgagaccagcctggtctacaaagtgaattccaggacagccagggctatacagagaaaccctgtcttgaaaaaaaaagagaaagaaaaaagaaaaaaaaaaatgaaagcagccacatctaaggactacgtggcacaggagagggtgagtccctgagagttcagctgctgccctgtctgttcctgtaaatggcagtggggtcatgggaaagtgaaggggctcaaggtattggacacttccaggataatcttttggacgcctcaccctgtgccaggaccaaggctgagcttggcaggctcagaacagggtgtcctgttcttccctgtctaaaacattcactctcagcttgctcacccttccccagacaaggaagctgcacagggtctggtgttcagatggctttggcttacagcaggtgtgggtgtggggtaggaggcagggggtaggggtgggggaagcctgtactatactcactatcctgtttctgaccctctaggactcctacagggttatgggagtggacaggcagtccagatctgagctgctgacccacaagcagtgccctgtgcctgccagaatccaaagccctgggaatgtccctctggtccccctctgtcccctgcagcccttcctgttgctcaaccttgcacagttccgacctgggggagagagggacagaaatcttgccaagtatttcaacagaatgtactggcaattacttcatggcttcctggacttggtaaaggatggactaccccgcccaacaggggggctggcagccaggtaggcccataaaaagcccgctggggagtcctcctcactctctgctcttcctcctccagcacacatcagacctagtagctgtggaaacca

SEQ ID NO: 6 - DNA, 1672 bp, Homo sapiens
  source 1..1672: mol_type = unassigned DNA
gtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagtagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggactgggcagggttctgaccctgtgggaccagagtggagggcgtagatggacctgaagtctccagggacaacagggcccaggtctcaggctcctagttgggcccagtggctccagcgtttccaaacccatccatccccagaggttcttcccatctctccaggctgatgtgtgggaactcgaggaaataaatctccagtgggagacggaggggtggccagggaaacggggcgctgcaggaataaagacgagccagcacagccagctcatgcgtaacggctttgtggagctgtcaaggcctggtctctgggagagaggcacagggaggccagacaaggaaggggtgacctggagggacagatccaggggctaaagtcctgataaggcaagagagtgccggccccctcttgccctatcaggacctccactgccacatagaggccatgattgacccttagacaaagggctggtgtccaatcccagcccccagccccagaactccagggaatgaatgggcagagagcaggaatgtgggacatctgtgttcaagggaaggactccaggagtctgctgggaatgaggcctagtaggaaatgaggtggcccttgagggtacagaacaggttcattcttcgccaaattcccagcaccttgcaggcacttacagctgagtgagataatgcctgggttatgaaatcaaaaagttggaaagcaggtcagaggtcatctggtacagcccttccttccctttttttttttttttttttgtgagacaaggtctctctctgttgcccaggctggagtggcgcaaacacagctcactgcagcctcaacctactgggctcaagcaatcctccagcctcagcctcccaaagtgctgggattacaagcatgagccaccccactcagccctttccttcctttttaattgatgcataataattgtaagtattcatcatggtccaaccaaccctttcttgacccaccttcctagagagagggtcctcttgattcagcggtcagggccccagacccatggtctggctccaggtaccacctgcctcatgcaggagttggcgtgcccaggaagctctgcctctgggcacagtgacctcagtggggtgaggggagctctccccatagctgggctgcggcccaaccccaccccctcaggctatgccagggggtgttgccaggggcacccgggcatcgccagtctagcccactccttcataaagccctcgcatcccaggagcgagcagagccagagcat

SEQ ID NO: 7 - DNA, 1610 bp, Mus musculus
  source 1..1610: mol_type = unassigned DNA
aactgagagtggaggggcacagaagagcccaagaggctccttaggttgtgtggagggtacaatatgtttgggctgagcaacccagagccagactttgtctggctggtaagagacagaggtgcctgctatcacaatccaagggtctgcttgaggcagagccagtgcaaaggatgtggttagagccagcctggtgtactgaagaggggcgaagagcttgagtaaggagtctcagcggtggtttgagaggcagggtggttaatggagtagctgcaggggagaatccttgggagggagcctgcaggacagagctttggtcaggaagtgatgggcatgtcactggaccctgtattgtctctgacttttctcaagtaggacaatgactctgcccagggagggggtctgtgacaaggtggaagggccagaggagaacttctgagaagaaaaccagaggccgtgaagaggtgggaagggcatgggattcagaacctcaggcccaccaggacacaaccccaggtccacagcagatgggtgaccttgcatgtctcagtcaccagcattgtgctccttgcttatcacgcttgggtgaaggaaatgacccaaatagcataaagcctgaaggccgggactaggccagctagggcttgcccttcccttcccagctgcactttccataggtcccaccttcagcagattagacccgcctcctgcttcctgcctccttgcctcctcactcatgggtctatgcccacctccagtctcgggactgaggctcactgaagtcccatcgaggtctggtctggtgaatcagcggctggctctgggccctgggcgaccagttaggttccgggcatgctaggcaatgaactctacccggaattgggggtgcggggaggcggggaggtctccaacccagccttttgaggacgtgcctgtcgctgcacggtgctttttatagacgatggtggcccattttgcagaagggaaagccggagccctctggggagcaaggtccccgcaaatggacggatgacctgagcttggttctgccagtccacttcccaaatccctcaccccattctagggactagggaaagatctcctgattggtcatatctgggggcctggccggagggcctcctatgattggagagatctaggctgggcgggccctagagcccgcctcttctctgcctggaggaggagcactgaccctaaccctctctgcacaagacccgagcttgtgcgcccttctgggagcttgctgcccctgtgctgactgctgacagctgactgacgctcgcagctagcaggtacttctgggttgctagcccagagccctgggccggtgaccctgttttccctacttcccgtctttgaccttgggtaagtttctttttcttttgtttttgagagaggcacccagatcctctccactacaggcagccgctgaaccttggatcctcagctcctgccctgggaactacagttcctgccctttttttcccaccttgagggaggttttccctgagtagcttcgactatcctggaacaagctttgtagaccagcctgggtctccggagagttgggattaaaggcgtgcaccaccacc

SEQ ID NO: 8 - DNA, 1387 bp, Homo sapiens
  source 1..1387: mol_type = unassigned DNA
ctctggtttcaagaccaatactcataacccccacatggaccaggcaccatcacacctgagcactgcacttagggtcaaagacctggccccacatctcagcagctatgtagactagctccagtcccttaatctctctcagcctcagtttcttcatctgcaaaacaggtctcagtttcgttgcaaagtatgaagtgctgggctgttactggtcaaagggaagagctgggaagagggtgcaaggtggggttgggctggagatgggctggagcagatagatggagggacctgaatggaggaagtaaaccaaggcccggtaacattgggactggacagagaacacgcagatcctctaggcaccggaagctaagtaacattgccctttctcctcctgtttgggactaggctgatgttgctgcctggaagggagccagcagaagggccccagcctgaagctgttaggtagaagccaaatccagggccagatttccaggaggcagcctcgggaagttgaaacacccggattcaggggtcaggaggcctgggcttctggcaccaaacggccagggacctactttccacctggagtcttgtaagagccactttcagcttgagctgcactttcgtcctccatgaaatgggggaggggatgctcctcacccaccttgcaaggttattttgaggcaaatgtcatggcgggactgagaattcttctgccctgcgaggaaatccagacatctctcccttacagacagggagactgaggtgaggcccttccaggcagagaaggtcactgttgcagccatgggcagtgccccacaggacctcgggtggtgcctctggagtctggagaagttcctaggggacctccgaggcaaagcagcccaaaagccgcctgtgagggtggctggtgtctgtccttcctcctaaggctggagtgtgcctgtggaggggtctcctgaactcccgcaaaggcagaaaggagggaagtaggggctgggacagttcatgcctcctccctgagggggtctcccgggctcggctcttggggccagagttcagggtgtctgggcctctctatgactttgttctaagtctttagggtggggctggggtctggcccagctgcaagggccccctcacccctgccccagagaggaacagccccgcacgggccctttaagaaggttgagggtgggggcaggtgggggagtccaagcctgaaacccgagcgggcgcgcgggtctgcgcctgccccgcccccggagttaagtgcgcggacacccggagccggcccgcgcccaggagcagagccgcgctcgctccactcagctcccagctcccaggactccgctggctcctcgcaagtcctgccgcccagcccgccggg

SEQ ID NO: 9 - DNA, 9232 bp, synthetic construct
  misc_feature 1..9232: note = vector construct
  source 1..9232: mol_type = other DNA
gatccggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcatgtacggtgggaggtctatataagcagagctcaataaaagagcccacaacccctcactcggggcgccagtcctccgattgactgagtcgcccgggtacccgtattcccaataaagcctcttgctgtttgcatccgaatcgtggtctcgctgttccttgggagggtctcctctgagtgattgactacccacgacgggggtctttcatttgggggctcgtccgggatttggagacccctgcccagggaccaccgacccaccaccgggaggtaagctggccagcaacttatctgtgtctgtccgattgtctagtgtctatgtttgatgttatgcgcctgcgtctgtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattcgatgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattcgctagcggatccggccgcctcggccaccggtcgccaccatcgccaccatgaccaaatcatacagcgagagcgggctgatgggcgagcctcagccccaaggtcccccaagctggacagatgagtgtctcagttctcaggacgaggaacacgaggcagacaagaaagaggacgagcttgaagccatgaatgcagaggaggactctctgagaaacgggggagaggaggaggaggaagatgaggatctagaggaagaggaggaagaagaagaggaggaggaggatcaaaagcccaagagacggggtcccaaaaagaaaaagatgaccaaggcgcgcctagaacgttttaaattaaggcgcatgaaggccaacgcccgcgagcggaaccgcatgcacgggctgaacgcggcgctggacaacctgcgcaaggtggtaccttgctactccaagacccagaaactgtctaaaatagagacactgcgcttggccaagaactacatctgggctctgtcagagatcctgcgctcaggcaaaagccctgatctggtctccttcgtacagacgctctgcaaaggtttgtcccagcccactaccaatttggtcgccggctgcctgcagctcaaccctcggactttcttgcctgagcagaacccggacatgcccccgcatctgccaaccgccagcgcttccttcccggtgcatccctactcctaccagtcccctggactgcccagcccgccctacggcaccatggacagctcccacgtcttccacgtcaagccgccgccacacgcctacagcgcagctctggagcccttctttgaaagccccctaactgactgcaccagcccttcctttgacggacccctcagcccgccgctcagcatcaatggcaacttctctttcaaacacgaaccatccgccgagtttgaaaaaaattatgcctttaccatgcactaccctgcagcgacgctggcagggccccaaagccacggatcaatcttctcttccggtgccgctgcccctcgctgcgagatccccatagacaacattatgtctttcgatagccattcgcatcatgagcgagtcatgagtgcccagcttaatgccatctttcacgattaggtttaaacgcggccgcgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcgagcttgttaacatcgataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttccgacttgtggtctcgctgccttgggagggtctcctctgagtgattgactacccgtcagcgggggtcttcacatgcagcatgtatcaaaattaatttggttttttttcttaagtatttacattaaatggccatagttgcattaatgaatcggccaacgcgcggggagaggcggtttgcgtattggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagtttgcggccggccgcaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaat

SEQ ID NO: 10 - DNA, 987 bp, Homo sapiens
  source 1..987: mol_type = unassigned DNA
atgactggagtctttgacagtctagtggctgatatgcactcgacccagatcgccgcctccagcacgtaccaccagcaccagcagcccccgagcggcggcggcgccggcccgggtggcaacagcagcagcagcagcagcctccacaagccccaggagtcgcccacccttccggtgtccaccgccaccgacagcagctactacaccaaccagcagcacccggcgggcggcggcggcggcgggggctcgccctacgcgcacatgggttcctaccagtaccaagccagcggcctcaacaacgtcccttactccgccaagagcagctatgacctgggctacaccgccgcctacacctcctacgctccctatggaaccagttcgtccccagccaacaacgagcctgagaaggaggaccttgagcctgaaattcggatagtgaacgggaagccaaagaaagtccggaaaccccgcaccatctactccagtttccagctggcggctcttcagcggcgtttccaaaagactcaatacttggccttgccggagcgagccgagctggcggcctctctgggcctcacccagactcaggtcaaaatctggttccagaaccgccggtccaagttcaagaagatgtggaaaagtggtgagatcccctcggagcagcaccctggggccagcgcttctccaccttgtgcttcgccgccagtctcagcgccggcctcctgggactttggtgtgccgcagcggatggcgggcggcggtggtccgggcagtggcggcagcggcgccggcagctcgggctccagcccgagcagcgcggcctcggcttttctgggcaactacccctggtaccaccagacctcgggatccgcctcacacctgcaggccacggcgccgctgctgcaccccactcagaccccgcagccgcatcaccaccaccaccatcacggcggcgggggcgccccggtgagcgcggggacgattttctaa

SEQ ID NO: 11 - protein, 328 aa, Homo sapiens
MTGVFDSLVADMHSTQIAASSTYHQHQQPPSGGGAGPGGNSSSSSSLHKPQESPTLPVSTATDSSYYTNQQHPAGGGGGGGSPYAHMGSYQYQASGLNNVPYSAKSSYDLGYTAAYTSYAPYGTSSSPANNEPEKEDLEPEIRIVNGKPKKVRKPRTIYSSFQLAALQRRFQKTQYLALPERAELAASLGLTQTQVKIWFQNRRSKFKKMWKSGEIPSEQHPGASASPPCASPPVSAPASWDFGVPQRMAGGGGPGSGGSGAGSSGSSPSSAASAFLGNYPWYHQTSGSASHLQATAPLLHPTQTPQPHHHHHHHGGGGAPVSAGTIF

SEQ ID NO: 12 - DNA, 999 bp, Mus musculus
  source 1..999: mol_type = unassigned DNA
atgactggagtctttgacagtctggtggctgatatgcactcgacccagatcaccgcctccagcacgtaccaccagcaccagcagcccccgagcggtgcgggcgccggccctggcggcaacagcaacagcagcagcagcaacagcagcctgcacaagccccaggagtcgccaaccctcccggtgtccacggctacggacagcagctactacaccaaccagcagcacccggcgggcggcggcggcgggggggcctcgccctacgcgcacatgggctcctaccagtaccacgccagcggcctcaacaatgtctcctactccgccaaaagcagctacgacctgggctacaccgccgcgtacacctcctacgcgccctacggcaccagttcgtctccggtcaacaacgagccggacaaggaagaccttgagcctgaaatccgaatagtgaacgggaagccaaagaaagtccggaaaccacgcaccatctactccagtttccagctggcggcccttcaacgacgcttccagaagacccagtatctggccctgccagagcgagccgagctggcggcgtccctgggcctcacccaaactcaggtcaaaatctggttccagaaccgccgatccaagttcaagaagatgtggaaaagcggcgagatacccaccgagcagcaccctggagccagcgcttctcctccttgtgcctccccgccggtctcggcgccagcatcctgggacttcggcgcgccgcagcggatggctggcggcggcccgggcagcggaggcggcggtgcgggcagctctggctccagcccgagcagcgccgcctcggcctttctgggaaactacccgtggtaccaccaggcttcgggctccgcttcacacctgcaggccacagcgccacttctgcatccttcgcagactccgcaggcgcaccatcaccaccatcaccaccaccacgcaggcgggggcgccccggtgagcgcggggacgattttctaa

SEQ ID NO: 13 - protein, 332 aa, Mus musculus
MTGVFDSLVADMHSTQITASSTYHQHQQPPSGAGAGPGGNSNSSSSNSSLHKPQESPTLPVSTATDSSYYTNQQHPAGGGGGGASPYAHMGSYQYHASGLNNVSYSAKSSYDLGYTAAYTSYAPYGTSSSPVNNEPDKEDLEPEIRIVNGKPKKVRKPRTIYSSFQLAALQRRFQKTQYLALPERAELAASLGLTQTQVKIWFQNRRSKFKKMWKSGEIPTEQHPGASASPPCASPPVSAPASWDFGAPQRMAGGGPGSGGGGAGSSGSSPSSAASAFLGNYPWYHQASGSASHLQATAPLLHPSQTPQAHHHHHHHHHAGGGAPVSAGTIF

SEQ ID NO: 14 - DNA, 2448 bp, Homo sapiens
  source 1..2448: mol_type = unassigned DNA
tgaaggaagaggaagaggaggagagggaggccagagccagaacagcccggcagcccgggcttcgggggagaacggcctgagccccgagcaagttgcctcgggagccctaatcctctcccgctggctcgccgagcggtcagtggcgctcagcggcggcgaggctgaaatatgataatcagaacagctgcgccgcgcgccctgcagccaatgggcgcggcgctcgcctgacgtccccgcgcgctgcgtcagaccaatggcgatggagctgagttggagcagagaagtttgagtaagagataaggaagagaggtgcccgagccgcgccgagtctgccgccgccgcagcgcctccgctccgccaactccgccggcttaaattggaatcctagatccgcgagggcgcggcgcagccgagcagcggctctttcagcattggcaaccccaggggccaatatttcccacttagccacagctccagcatcctctctgtgggctgttcaccagctgtacaaccaccatttcactgtggacattactccctcttacagatatgggagacatgggagatccaccaaaaaaaaaacgtctgatttccctatgtgttggttgcggcaatcagattcacgatcagtatattctgagggtttctccggatttggaatggcatgcggcatgtttgaaatgtgcggagtgtaatcagtatttggacgagagctgtacatgctttgttagggatgggaaaacctactgtaaaagagattatatcaggttgtacgggatcaaatgcgccaagtgcagcatcggcttcagcaagaacgacttcgtgatgcgtgcccgctccaaggtgtatcacatcgagtgtttccgctgtgtggcctgcagccgccagctcatccctggggacgaatttgcgcttcgggaggacggtctcttctgccgagcagaccacgatgtggtggagagggccagtctaggcgctggcgacccgctcagtcccctgcatccagcgcggccactgcaaatggcagcggagcccatctccgccaggcagccggccctgcggccccacgtccacaagcagccggagaagaccacccgcgtgcggactgtgctgaacgagaagcagctgcacaccttgcggacctgctacgccgcaaacccgcggccagatgcgctcatgaaggagcaactggtagagatgacgggcctcagtccccgtgtgatccgggtctggtttcaaaacaagcggtgcaaggacaagaagcgaagcatcatgatgaagcaactccagcagcagcagcccaatgacaaaactaatatccaggggatgacaggaactcccatggtggctgccagtccagagagacacgacggtggcttacaggctaacccagtggaagtacaaagttaccagccaccttggaaagtactgagcgacttcgccttgcagagtgacatagatcagcctgcttttcagcaactggtcaatttttcagaaggaggaccgggctctaattccactggcagtgaagtagcatcaatgtcctctcaacttccagatacacctaacagcatggtagccagtcctattgaggcatgaggaacattcattctgtattttttttccctgttggagaaagtgggaaattataatgtcgaactctgaaacaaaagtatttaacgacccagtcaatgaaaactgaatcaagaaatgaatgctccatgaaatgcacgaagtctgttttaatgacaaggtgatatggtagcaacactgtgaagacaatcatgggattttactagaattaaacaacaaacaaaacgcaaaacccagtatatgctattcaatgatcttagaagtactgaaaaaaaaagacgtttttaaaacgtagaggatttatattcaaggatctcaaagaaagcattttcatttcactgcacatctagagaaaaacaaaaatagaaaattttctagtccatcctaatctgaatggtgctgtttctatattggtcattgccttgccaaacaggagctccagcaaaagcgcaggaagagagactggcctccttggctgaaagagtcctttcaggaaggtggagctgcattggtttgatatgtttaaagttgactttaacaaggggttaattgaaatcctgggtctcttggcctgtcctgtagctggtttattttttactttgccccctccccactttttttgagatccatcctttatcaagaagtctgaagcgactttaaaggtttttgaattcagatttaaaaaccaacttataaagcattgcaacaaggttacctctattttgccacaagcgtctcgggattgtgtttgacttgtgtctgtccaagaacttttcccccaaagatgtgtatagttattggttaaaatgactgttttctctctctatggaaataaaaaggaaaaaaaaaaaaaaa

SEQ ID NO: 15 - protein, 349 aa, Homo sapiens
MGDMGDPPKKKRLISLCVGCGNQIHDQYILRVSPDLEWHAACLKCAECNQYLDESCTCFVRDGKTYCKRDYIRLYGIKCAKCSIGFSKNDFVMRARSKVYHIECFRCVACSRQLIPGDEFALREDGLFCRADHDVVERASLGAGDPLSPLHPARPLQMAAEPISARQPALRPHVHKQPEKTTRVRTVLNEKQLHTLRTCYAANPRPDALMKEQLVEMTGLSPRVIRVWFQNKRCKDKKRSIMMKQLQQQQPNDKTNIQGMTGTPMVAASPERHDGGLQANPVEVQSYQPPWKVLSDFALQSDIDQPAFQQLVNFSEGGPGSNSTGSEVASMSSQLPDTPNSMVASPIEA

SEQ ID NO: 16 - DNA, 1307 bp, Mus musculus
  source 1..1307: mol_type = unassigned DNA
caactccgccggcttaaatcggactcccagatctgcgagggcgcggcgcagccgggcagctgtttcccccagttttggcaaccccgggggccactatttgccacctagccacagcaccagcatcctctctgtgggctattcaccaattgtccaaccaccatttcactgtggacgttactccctcttacagatatgggagacatgggcgatccaccaaaaaaaaaacgtctgatttccctgtgtgttggttgcggcaatcaaattcacgaccagtatattctgagggtttctccggatttggagtggcatgcagcatgtttgaaatgtgcggagtgtaatcagtatttggacgaaagctgtacgtgctttgttagggatgggaaaacctactgtaaaagagattatatcaggttgtacgggatcaaatgcgccaagtgcagcataggcttcagcaagaacgacttcgtgatgcgcgcccgctctaaggtgtaccacatcgagtgtttccgctgtgtagcctgcagccgacagctcatcccgggagacgaattcgccctgcgggaggatgggcttttctgccgtgcagaccacgatgtggtggagagagccagcctgggagctggagaccctctcagtcccttgcatccagcgcggcctctgcaaatggcagccgaacccatctcggctaggcagccagctctgcggccgcacgtccacaagcagccggagaagaccacccgagtgcggactgtgctcaacgagaagcagctgcacaccttgcggacctgctatgccgccaaccctcggccagatgcgctcatgaaggagcaactagtggagatgacgggcctcagtcccagagtcatccgagtgtggtttcaaaacaagcggtgcaaggacaagaaacgcagcatcatgatgaagcagctccagcagcagcaacccaacgacaaaactaatatccaggggatgacaggaactcccatggtggctgctagtccggagagacatgatggtggtttacaggctaacccagtagaggtgcaaagttaccagccgccctggaaagtactgagtgacttcgccttgcaaagcgacatagatcagcctgcttttcagcaactggtcaatttttcagaaggaggaccaggctctaattctactggcagtgaagtagcatcgatgtcctcgcagctcccagatacacccaacagcatggtagccagtcctattgaggcatgaggaacattcattcagatgttttgttttgttttgttttgtttttttcccctgttggagaaagtggg

SEQ ID NO: 17 - protein, 349 aa, Mus musculus; identical to SEQ ID NO: 15